(19) United States
(12) Patent Application Publication
(10) Pub. No.: US 2026/0337689 A1
(43) Pub. Date: Oct. 29, 2026

(54) METHODS AND SYSTEMS FOR DETERMINING A DIAGNOSTIC GENE STATUS

(71) Applicant: Foundation Medicine, Inc., Boston, MA (US)

(72) Inventors: Karthikeyan MURUGESAN, Boston, MA (US); Ethan S. SOKOL, Somerville, MA (US)

(73) Assignee: Foundation Medicine, Inc., Boston, MA (US)

(21) Appl. No.: 18/997,457

(22) PCT Filed: Jul. 17, 2023
(86) PCT No.: PCT/US2023/070329

§ 371 Date: Jan. 21, 2025

Related U.S. Application Data
(60) Provisional application No. 63/391,417, filed on Jul. 22, 2022.

(51) Int. Cl. C12Q 1/6886; G16B 20/20; G16B 40/20; G16B 40/30

(52) U.S. Cl. CPC C12Q 1/6886; G16B 20/20; G16B 40/20; G16B 40/30; C12Q 2600/156

(57) ABSTRACT

Embodiments of the present disclosure are directed to systems and methods comprising receiving training data comprising training values for a plurality of training input features associated with a gene receptor status in a plurality of training samples, training a statistical model based on the training data, obtaining a set of input features associated with the gene receptor status and a sample type, receiving sequence read data associated with a sample from an individual, determining values for one or more input features associated with the gene receptor status based on the sequence read data, wherein the one or more input features to be evaluated are specified based on a sample type of the sample, inputting the values for the one or more input features into the trained statistical model and predicting the gene receptor of the individual based on an output of the trained statistical model.

### CROSS-REFERENCE TO RELATED APPLICATION

This application claims the benefit of U.S. Provisional Application No. 63/391,417, filed Jul. 22, 2022, the contents of which are hereby incorporated by reference in its entirety.

### FIELD OF THE DISCLOSURE

The present disclosure relates generally to methods and systems for analyzing genomic profiling data, and more specifically to methods and systems for determining a diagnostic gene status in a sample.

### BACKGROUND

The diagnostic gene status (e.g., a hormone receptor status or receptor gene status) of a patient can be useful for informing the prognosis and treatment options for patients. For example, breast cancer cells that express estrogen or progesterone female hormone receptors (e.g., on the cell surface or inside the cell) can be stimulated and subsequently grow and propagate in the presence of the estrogen and progesterone hormones, respectively. About 70%-80% of breast cancer patients have hormone receptor positivity (estrogen receptor (ER) positive status, and/or progesterone receptor (PR) positive status). Hormone receptor positive patients may respond well to treatment with hormone therapy in both the adjuvant and the neo-adjuvant setting. For example, hormone therapies may be used to block the interaction between the hormone and a respective receptor (e.g., to block the interaction between estrogen and estrogen receptors) and/or to reduce hormone levels (e.g., to reduce the estrogen levels).

Accordingly, determining the diagnostic gene status or receptor status of an individual can inform the prognostic and treatment options. As used herein, diagnostic gene status may refer to a receptor status or hormone status corresponding to a gene that has a known pathogenic or likely pathogenic effect. For example, for breast cancer patients, the ER status, PR status and human epidermal growth factor 2 (HER2) status can inform the prognosis and treatment options for an individual. Generally, the receptor status of breast cancer patients may be determined using an immunohistochemistry (IHC) test based on a tissue biopsy. The IHC test can provide a semi-quantitative measurement of ER/PR positive tumor nuclei in stained histologic tissue sections. However, obtaining tissue samples is an invasive and painful process, and in some situations, it may not be possible to obtain a tissue sample. Accordingly, there is a need to provide an accurate system to determine a receptor status that can be run on both solid and liquid biopsy samples.

### BRIEF SUMMARY

Disclosed herein are methods and systems for predicting a diagnostic gene status (e.g., a receptor status). The diagnostic gene status, such as the hormone receptor status of a patient, can be useful for informing the treatment options for an individual. For example, embodiments of the methods disclosed herein may be used to predict a hormone receptor status (e.g., estrogen receptor (ER) status, progesterone receptor (PR) status, androgen receptor status) or a status of a gene (e.g., human epidermal growth factor receptor 2 (HER2) status). In one or more examples, the prediction method may rely on evaluating a predetermined set of features (e.g., genomic features) to predict the receptor status. In some embodiments, the methods of the present disclosure may be used to assess the presence or absence other genomic alterations and complex biomarker signatures in addition to predicting receptor status to get a holistic view of the genomic landscape driving the growth tumor, as opposed to tests that may provide information only for a single receptor biomarker. In one or more examples, the genomic features to be evaluated may provide granularity with respect to the presence of specific genomic alterations that allow specific and meaningful insights from such data to be applied to the determination of receptor status. Moreover, the breadth of features included in the statistical model may enhance the accuracy of the predictions. To the extent that specific examples of predicting a receptor status are described with respect to particular receptors, a skilled artisan will understand that methods described herein are not limited to the particular receptors described.

In one or more examples, embodiments of the present disclosure may be used to predict the receptor status through genomic sequencing data obtained from either a solid biopsy specimen or a liquid biopsy specimen. As opposed to an immunohistochemistry (IHC) test, which is routinely run-on only solid biopsy specimens, embodiments of the present disclosure may rely on genomic profile testing, and in this manner are able to detect receptor status in both solid and liquid tumor (e.g., blood-based) specimens. The ability to predict a receptor status via either solid or liquid samples permits a prediction of a receptor status when solid samples are inaccessible. For example, traditional solid biopsies may suffer from tissue inaccessibility due to the anatomical site of the tumor, poor quality of the tissue sample, and/or an insufficient amount of the tissue sample. Moreover, the procedures used to obtain such tissue samples can be high-risk, expensive, and time-consuming, and are generally not be repeated very often on the individual. Compared to traditional solid biopsies, liquid biopsies are quick, less invasive, have a high throughput, are convenient to the patient, potentially less expensive, can be run multiple times for disease and/or treatment monitoring, and can provide real-time, tissue-site agnostic holistic information about the tumor.

In some instances, for example, methods are described that comprise providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of reads that represent the captured nucleic acid molecules; receiving, at one or more processors, sequence read data for the plurality of reads; receiving, using one or more processors, training data comprising training values for a plurality of training input features associated with a receptor gene status in a plurality of training samples; training, using the one or more processors, a statistical model based on the training data; obtaining, using the one or more processors, a set of input features associated with the receptor gene status, wherein obtaining the set of input features comprises omitting input features with a low prevalence and high correlation to the statistical model; determining, using the one or more processors, values for one or more input features associated with the receptor gene status based on the sequence read data, wherein the one or more input features to be evaluated are specified based on a sample type of the sample; inputting, using the one or more processors, the values for the one or more input features into the trained statistical model; and predicting, using the one or more processors, the receptor gene status of the subject based on an output of the trained statistical model.

In one or more examples, the receptor gene status corresponds to an estrogen receptor status, a progesterone receptor status, an androgen receptor status, or a human epidermal growth factor receptor 2 status. In any of the examples of this disclosure, the receptor gene status comprises a hormone receptor status. In any of the examples of this disclosure, the method further comprises applying the trained statistical model to the values for the one or more input features to obtain an output indicative of the receptor gene status. In any of the examples of this disclosure, the sample type is indicative of a solid sample and the set of input features comprises genomic alteration features, complex mutational signatures, chromosomal instability features, clinicopathological features, clinical features, additional features, or a combination thereof.

In any of the examples of this disclosure, the genomic alteration features comprise one or more genomic alterations and a type of alteration associated with the one or more genomic alterations.

In any of the examples of this disclosure, the sample type is indicative of a liquid sample and the set of input features comprises genomic alteration features, clinicopathological features, clinical features, tumor fraction features, fragmentomic features, additional features, or a combination thereof. In one or more examples of this disclosure, the genomic alteration features comprise one or more genomic alterations and a type of alteration associated with the one or more genomic alterations.

In any of the examples of tis disclosure, sequence read data derived from a liquid sample is associated with a first set of one or more input features and sequence read data derived from a solid sample is associated with a second set of one or more input features.

In any of the examples of this disclosure, the subject is suspected of having or is determined to have cancer. In one or more examples of this disclosure, the cancer comprises breast cancer (HER2 overexpressed/amplified), breast cancer (HER2+), breast cancer (HR+, HER2−), and prostate cancer. In one or more examples of this disclosure, the method further comprises treating the subject with an anti-cancer therapy. In one or more examples of this disclosure, the anti-cancer therapy comprises a targeted anti-cancer therapy. In one or more examples of this disclosure, the targeted anti-cancer therapy comprises alpelisib (Piqray), CDK4/6 inhibitors, or any combination thereof.

In any of the examples of this disclosure, the method further comprises obtaining the sample from the subject. In any of the examples of this disclosure, the sample comprises a tissue biopsy sample, a liquid biopsy sample, or a normal control. In one or more examples, the set of features differs between a tissue biopsy sample and a liquid biopsy. In one or more examples, of this disclosure, the sample is a liquid biopsy sample and comprises blood, plasma, cerebrospinal fluid, sputum, stool, urine, or saliva. In one or more examples of this disclosure, the sample is a liquid biopsy sample and comprises circulating tumor cells (CTCs). In one or more examples of this disclosure, the sample is a liquid biopsy sample and comprises cell-free DNA (cfDNA), circulating tumor DNA (ctDNA), or any combination thereof.

In any of the examples of this disclosure, the plurality of nucleic acid molecules comprises a mixture of tumor nucleic acid molecules and non-tumor nucleic acid molecules. In one or more examples of this disclosure, the tumor nucleic acid molecules are derived from a tumor portion of a heterogeneous tissue biopsy sample, and the non-tumor nucleic acid molecules are derived from a normal portion of the heterogeneous tissue biopsy sample. In one or more examples of this disclosure, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In any of the examples of this disclosure, the one or more adapters comprise amplification primers, flow cell adaptor sequences, substrate adapter sequences, or sample index sequences. In any of the examples of this disclosure, the captured nucleic acid molecules are captured from the amplified nucleic acid molecules by hybridization to one or more bait molecules. In one or more examples of this disclosure, the one or more bait molecules comprise one or more nucleic acid molecules, each comprising a region that is complementary to a region of a captured nucleic acid molecule.

In any of the examples of this disclosure, amplifying nucleic acid molecules comprises performing a polymerase chain reaction (PCR) amplification technique, a non-PCR amplification technique, or an isothermal amplification technique. In any of the examples of this disclosure, the sequencing comprises use of a massively parallel sequencing (MPS) technique, whole genome sequencing (WGS), whole exome sequencing, targeted sequencing, direct sequencing, or Sanger sequencing technique. In one or more examples of this disclosure, the sequencing comprises massively parallel sequencing, and the massively parallel sequencing technique comprises next generation sequencing (NGS). In any of the examples of this disclosure, the sequencer comprises a next generation sequencer.

In any of the examples of this disclosure, one or more of the plurality of sequencing reads overlap one or more gene loci within one or more subgenomic intervals in the sample. In one or more examples of this disclosure, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In one or more examples of this disclosure, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In one or more examples of this disclosure, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In any of the examples of this disclosure, the method further comprises generating, by the one or more processors, a report indicating a receptor gene status of the sample. In one or more examples of this disclosure, the method further comprises transmitting the report to a healthcare provider. In one or more examples of this disclosure, the report is transmitted via a computer network or a peer-to-peer connection.

In some instances, for example, methods are described that comprise receiving, using one or more processors, training data comprising training values for a plurality of training input features associated with a receptor gene status in a plurality of training samples; training, using the one or more processors, a statistical model based on the training data; obtaining, using the one or more processors, a set of input features associated with the receptor gene status and a sample type, wherein obtaining the set of input features comprises omitting input features with a low prevalence and high correlation to the statistical model; receiving, using one or more processors, sequence read data associated with a sample from an individual; determining, using the one or more processors, values for one or more input features associated with the receptor gene status based on the sequence read data, wherein the one or more input features to be evaluated are specified based on a sample type of the sample; inputting, using the one or more processors, the values for the one or more input features into the trained statistical model; and predicting, using the one or more processors, the receptor gene status of the individual based on an output of the trained statistical model.

In any of the examples of this disclosure, the receptor gene status corresponds to an estrogen receptor status, a progesterone receptor status, an androgen receptor status, or a human epidermal growth factor receptor 2 status. In any of the examples of this disclosure, the method further comprises applying the trained machine learning model to the values for the one or more input features to obtain an output indicative of the receptor gene status. In any of the examples of tis disclosure, sequence read data derived from a liquid sample is associated with a first set of one or more input features and sequence read data derived from a solid sample is associated with a second set of one or more input features.

In any of the examples of this disclosure, the sequence read data for the individual is derived from a solid sample. In one or more examples of this disclosure, the sequence read data for the individual is derived from a biopsy sample. In one or more examples of this disclosure, the set of input features comprises genomic alteration features, complex mutational signatures, chromosomal instability features, clinicopathological features, clinical features, additional features, or a combination thereof.

In one or more examples of this disclosure, the one or more input features are associated with one or more genomic alteration features. In one or more examples of this disclosure, the one or more genomic alteration features comprise a presence of a predetermined short variant, an absence of a predetermined short variant, a copy number alteration, a rearrangement alteration, an amplification, a deletion, or a combination thereof. In one or more examples of this disclosure, the predetermined short variant comprises a point mutation, an insertion, or a deletion.

In one or more examples of this disclosure, the one or more genomic alteration features comprise CCNE1, EGFR, ERBB2, ERBB2 Amplification, ESR1, FGF3, FGFR1, GATA3, MAP2K4, MYC, PIK3CA, PTEN, RB1, TP53, ZNF703, or a combination thereof. In one or more examples of this disclosure, the one or more genomic alteration features comprise BRCA1, TP53, MYC, ERBB2, ERBB2 Amplification, ERBB2 Short Variant, PIK3CA, ESR1, TBX3, CDH1, ZNF703, GATA3, FGF3, CCNE1, RB1, FGFR1, PTEN, EGFR, MAP2K4, or a combination thereof.

In one or more examples of this disclosure, the one or more genomic alteration features comprise AKT1, AKT2, AKT3, APC, AR, ARFRP1, ARID1A, ATM, AURKA, BAP1, BCL2L1, BCL2L2, BRAF, BRCA1, BRCA2, BRD4, CBFB, CCND3, CCNE1, CDH1, CDK12, CDK4, CDK6, CDKN2A, CDKN2B, CHEK2, CREBBP, CTNNA1, DNMT3A, EGFR, EMSY, EP300, ERBB2, ERBB2 Amplification, ERBB2 Short Variant, ERBB3, ESR1, FANCA, FAS, FBXW7, FGF10, FGF12, FGF3, FGF6, FGFR1, FGFR2, FGFR4, GATA3, GNAS, HGF, IGFIR, IKBKE, IRS2, JAK2, KDM5A, KDM6A, KDR, KEL, KIT, KMT2D, KRAS, LYN, MAP2K4, MAP3K1, MCL1, MDM2, MDM4, MSH6, MUTYH, MYC, MYCL, NF1, NFKBIA, NKX2 1, NOTCH1, NOTCH2, NOTCH3,, TRK1, PALB2, PBRM1, PDCD1LG2, PDGFRA, PIK3C2B, PIK3CA, PIK3CB, PIK3R1, PRKCI, PTEN, RAF1, RB1, RET, RICTOR, ROS1, RPTOR, SETD2, SF3B1, SMAD4, SMARCA4, SOX2, SPEN, SRC, STK11, TBX3, TERC, TET2, TP53, TSC1, VEGFA, ZNF217, ZNF703, or a combination thereof.

In one or more examples of this disclosure, the one or more input features are associated with one or more complex mutational signatures. In one or more examples of this disclosure, the one or more complex mutational signatures comprise a genome-wide loss of heterozygosity (gLOH) quantification, trinucleotide signatures, insertion signatures, deletion signatures, copy number signatures, or a combination thereof.

In one or more examples of this disclosure, the one or more input features are associated with one or more chromosomal instability features. In one or more examples of this disclosure, the one or more chromosomal instability features is indicative of aneuploidy. In one or more examples of this disclosure, the one or more chromosomal instability features is indicative of chromosome gain, chromosome not gain, chromosome loss, chromosome not loss, or a combination thereof. In one or more examples of this disclosure, the one or more chromosomal instability features comprises a total aneuploidy count.

In one or more examples of this disclosure, the one or more chromosomal instability features comprise chromosome 1q loss status, chromosome 1p loss status, chromosome 3p loss status, chromosome 4p loss status, chromosome 5q loss status, chromosome 9p loss status, chromosome 10q loss status, chromosome 11q loss status, chromosome 12q loss status, chromosome 14q loss status, chromosome 15q loss status, chromosome 16p loss status, chromosome 16q loss status, chromosome 17q loss status, chromosome 19p loss status, chromosome 21q loss status, chromosome 22q loss status, chromosome 1q gain status, chromosome 2p gain status, chromosome 5p gain status, chromosome 5q gain status, chromosome 6q gain status, chromosome 7q gain status, chromosome 9p gain status, chromosome 10p gain status, chromosome 12q gain status, chromosome 16p gain status, chromosome 18p gain status, chromosome 20q gain status chromosome 21p gain status, chromosome 21q gain status.

In one or more examples of this disclosure, the one or more chromosomal instability features comprise chromosome 1p gain status, chromosome 1q gain status, chromosome2p gain status, chromosome 2q gain status, chromosome 3p gain status, chromosome 3q gain status, chromosome 4p gain status, chromosome 4q gain status, chromosome 5p gain status, chromosome 5q gain status, chromosome 6p gain status, chromosome 6q gain status, chromosome 7p gain status, chromosome 7q gain status, chromosome 8p gain status, chromosome 8q gain status, chromosome 9p gain status, chromosome 9q gain status, chromosome 10p gain status, chromosome 10q gain status, chromosome 11p gain status, chromosome 11q gain status, chromosome 12p gain status, chromosome 12q gain status, chromosome 13q gain status, chromosome 14q gain status, chromosome 15q gain status, chromosome 16p gain status, chromosome 16q gain status, chromosome 17p gain status, chromosome 17q gain status, chromosome 18p gain status, chromosome 18q gain status, chromosome 19p gain status, chromosome 19q gain status, chromosome 20p gain status, chromosome 20q gain status, chromosome 21p gain status, chromosome 21q gain status, chromosome 22q gain status, chromosome 1p loss status, chromosome 2p loss status, chromosome 2q loss status, chromosome 3p loss status, chromosome 3q loss status, chromosome 4p loss status, chromosome 4q loss status, chromosome 5p loss status, chromosome 5q loss status, chromosome 6p loss status, chromosome 6q loss status, chromosome 7p loss status, chromosome 7q loss status, chromosome 8p loss status, chromosome 8q loss status, chromosome 9p loss status, chromosome 9q loss status, chromosome 10p loss status, chromosome 10q loss status, chromosome 11p loss status, chromosome 11q loss status, chromosome 12p loss status, chromosome 12q loss status, chromosome 13q loss status, chromosome 14q loss status, chromosome 15q loss status, chromosome 16p loss status, chromosome 16q loss status, chromosome 17p loss status, chromosome 17q loss status, chromosome 18p loss status, chromosome 18q loss status, chromosome 19p loss status, chromosome 19q loss status, chromosome 20p loss status, chromosome 20q loss status, chromosome 21p loss status, chromosome 21q loss status, chromosome 22q loss status.

In one or more examples of this disclosure, the one or more input features are associated with one or more clinicopathological features. In one or more examples of this disclosure, the one or more clinicopathological features comprise an age of the individual, a sex of the individual, a disease diagnosis of the individual, a tumor status of the individual, a tumor type, an anatomical sub-classification of a tumor, or a combination thereof.

In one or more examples of this disclosure, the one or more input features are associated with one or more clinical features. In one or more examples of this disclosure, the one or more clinical features comprise an ancestry of the individual, a family history of the individual, a stage of the disease, one or more laboratory-based test results, one or more lifestyle habits, a diabetes status, an obesity status, an indication of body vitamin levels, or a combination thereof.

In one or more examples of this disclosure, wherein the one or more input features are associated with a tumor mutational burden. In one or more examples of this disclosure, the one or more input features are associated with a germline status. In one or more examples of this disclosure, the one or more input features are associated with homologous repair deficiency (HRD) signature. In one or more examples of this disclosure, the one or more input features are associated with a methylation signature, a mRNA expression level, a miRNA expression level, proteomics, cosmic mutation signatures, immunohistochemical markers, genetic predispositions, cell adhesion biomarkers, saliva based biomarkers, enzyme based biomarkers, a diagnostic image, or a combination thereof.

In one or more of the examples of this disclosure, the sequence read data for the individual is derived from a liquid sample. In one or more of the examples of this disclosure, the sequence read data for the individual is derived from a liquid biopsy sample. In one or more of the examples of this disclosure, the set of input features comprises genomic alteration features, clinicopathological features, clinical features, tumor fraction features, fragmentomic features, additional features, or a combination thereof.

In one or more of the examples of this disclosure, the one or more input features are associated with one or more genomic alteration features. In one or more of the examples of this disclosure, the one or more genomic alteration features comprise a presence of a predetermined short variant, an absence of a predetermined short variant, a rearrangement alteration, an insertion, a deletion, or a combination thereof.

In one or more of the examples of this disclosure, the one or more genomic alteration features comprise TP53, ESR1, PIK3CA, or a combination thereof. In one or more of the examples of this disclosure, the one or more genomic alteration features comprise CDH1, BRCA1, AKT1, BRCA2, CDKN2A, ERBB2, KRAS, NF1, PTEN, or a combination thereof.

In one or more of the examples of this disclosure, the one or more input features are associated with one or more clinicopathological features. In one or more of the examples of this disclosure, the one or more clinicopathological features comprise an age of the individual, a sex of the individual, a disease diagnosis of the individual, a tumor type, or a combination thereof.

In one or more of the examples of this disclosure, the one or more input features are associated with one or more clinical features. In one or more of the examples of this disclosure, the one or more clinical features comprise an ancestry of the individual, a stage of the disease, one or more laboratory-based test results, one or more lifestyle habits, a diabetes status, an obesity status, an indication of body vitamin levels, or a combination thereof.

In one or more of the examples of this disclosure, the one or more input features are associated with a tumor mutational burden. In one or more of the examples of this disclosure, the one or more input features are associated with trinucleotide signatures, insertion signatures, deletion signatures, copy number signatures, or a combination thereof. In one or more of the examples of this disclosure, the one or more input features are associated with the HRD signature.

In one or more of the examples of this disclosure, the one or more input features are associated with one or more fragmentomic features, the one or more fragmentomic features associated with a plurality of reads obtained from the sequence read data. In one or more of the examples of this disclosure, the one or more fragmentomic features comprise lengths of the plurality reads, regions associated with the plurality of reads, or a combination thereof. In one or more of the examples of this disclosure, the one or more fragmentomic features comprise an amount of a fragment having a specified length, a mean fragment length of the plurality of reads, a median fragment length of the plurality of reads, an interquartile range of fragment lengths of the plurality of reads, a distribution of fragment lengths of the plurality of reads, one or more peaks of fragment lengths for the plurality of reads, a fragment length for the plurality of reads, a start position of a fragment for the plurality of reads, and an end position of a fragment for the plurality of reads.

In one or more of the examples of this disclosure, the one or more input features are associated with an estimated tumor fraction. In one or more of the examples of this disclosure, the one or more input features are associated with a methylation signature, a mRNA expression level, a miRNA expression level, proteomics, cosmic mutation signatures, immunohistochemical markers, genetic predispositions, cell adhesion biomarkers, saliva based biomarkers, enzyme based biomarkers, a diagnostic image, or a combination thereof.

In one or more of the examples of this disclosure, the output of the trained statistical model is indicative of a receptor status. In one or more of the examples of this disclosure, the output of the trained statistical model comprises a first score indicative of a probability of a positive receptor gene status. In one or more of the examples of this disclosure, the output of the trained statistical model comprises a second score indicative of a probability of a negative receptor gene status.

In any of the examples of this disclosure, the training set of input features associated with the training values for the input features is different from the values for one or more input features input into the trained statistical model. In any of the examples of this disclosure, the training data further comprises receptor gene statuses corresponding to each of the plurality of training samples. In one or more examples of this disclosure, the method further comprises determining, using the one or more processors, weights associated with the training values for the plurality of training input features based on the training; and filtering, using the one or more processors, the plurality of training input features based on the weights; wherein filtering the plurality of training input features comprises removing low prevalence training values, highly correlated training values, or a combination thereof. In one or more examples of this disclosure, the method further comprises weighting, using the one or more processors, the training values for the one or more input features based on the weights, wherein predicting the receptor gene status is based on the weighted values for the one or more input features.

In any of the examples of this disclosure, the receptor gene status comprises a hormone receptor status. In any of the examples of this disclosure, the trained statistical model is a trained machine learning model, a part of a machine learning process, or a combination thereof. In any of the examples of this disclosure, the trained statistical model includes an artificial intelligence learning model. In any of the examples of this disclosure, the trained statistical model comprises a random forest model.

In any of the examples of this disclosure, the trained statistical model comprises at least one of an extreme gradient boosting model, a logistic regression model, an elastic net model, a ridge regression model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.

In any of the examples of this disclosure, the method further comprises: inputting, using the one or more processors, a diagnostic image taken of the individual into a second statistical model; and determining, using the one or more processors, a third score indicative of a tumor classification based on the diagnostic image, wherein predicting the receptor gene status is further based on the third score. In one or more of the examples of this disclosure, the second statistical model is at least one of a deep learning model, a convolutional neural network model, a recurrent neural network model, and an auto-encoder model.

In one or more of the examples of this disclosure, the method further comprises training the second statistical model, wherein the second statistical model is trained by: receiving, using the one or more processors, training data including a plurality of training diagnostic images; and training, using the one or more processors, the second statistical model based on the training data. In one or more of the examples of this disclosure, the diagnostic image comprises at least one of a histopathological image, a radiological image, a magnetic resonance image, an ultrasound image, an X-ray image, a bone scan, a CT scan, a PET scan, or a combination thereof.

In any of the examples of this disclosure, the method further comprises assigning, using the one or more processors, a therapy for the individual based on the predicted receptor gene status. In any of the examples of this disclosure, the method further comprises determining, using the one or more processors, a treatment decision for the individual based on the predicted receptor gene status. In any of the examples of this disclosure, the method further comprises administering, using the one or more processors, a treatment to the individual based on the predicted receptor gene status. In any of the examples of this disclosure, the method further comprises monitoring, using the one or more processors, a prognosis of the individual based on the predicted receptor gene status. In any of the examples of this disclosure, the method further comprises predicting, using the one or more processors, one or more clinical outcomes based on the predicted receptor gene status.

In some instances, methods of the present disclosure are directed to methods for diagnosing a disease, the method comprising: diagnosing that a subject has the disease based on a determination of a receptor gene status for a sample from the subject, wherein the receptor gene status is determined according to the method of any one of the methods described above.

In some instances, methods of the present disclosure are directed to methods for selecting an anti-cancer therapy, the method comprising: responsive to determining a receptor gene status for a sample from a subject, selecting an anti-cancer therapy for the subject, wherein the receptor gene status is determined according to the method of any one of the methods described above.

In some instances, methods of the present disclosure are directed to methods of treating a cancer in a subject, comprising: responsive to determining a receptor gene status for a sample from the subject, administering an effective amount of an anti-cancer therapy to the subject, wherein the receptor gene status is determined according to the method of any one of the methods described above.

In some instances, methods of the present disclosure are directed to methods for monitoring cancer progression or recurrence in a subject, the method comprising: determining a first receptor gene status in a first sample obtained from the subject at a first time point according to the method of any one of the methods described above; determining a second receptor gene status in a second sample obtained from the subject at a second time point; and comparing the first receptor gene status to the second receptor gene status, thereby monitoring the cancer progression or recurrence. In one or more of the examples of this disclosure, the second receptor gene status for the second sample is determined according to the method of any one of the methods described above.

In one or more of the examples of this disclosure, the method further comprises selecting an anti-cancer therapy for the subject in response to the cancer progression. In one or more of the examples of this disclosure, the method further comprises administering an anti-cancer therapy to the subject in response to the cancer progression. In one or more of the examples of this disclosure, the method further comprises adjusting an anti-cancer therapy for the subject in response to the cancer progression. In one or more of the examples of this disclosure, the method further comprises adjusting a dosage of the anti-cancer therapy or selecting a different anti-cancer therapy in response to the cancer progression. In one or more of the examples of this disclosure, the method further comprises administering the adjusted anti-cancer therapy to the subject.

In one or more of the examples of this disclosure the first time point is before the subject has been administered an anti-cancer therapy, and wherein the second time point is after the subject has been administered the anti-cancer therapy. In one or more of the examples of this disclosure, the subject has a cancer, is at risk of having a cancer, is being routine tested for cancer, or is suspected of having a cancer.

In one or more of the examples of this disclosure, the cancer is a solid tumor. In one or more of the examples of this disclosure, the cancer is a breast cancer. In one or more of the examples of this disclosure, the anti-cancer therapy comprises chemotherapy, radiation therapy, immunotherapy, a targeted therapy, or surgery.

In any of the examples of this disclosure, the method further comprises determining, identifying, or applying the receptor gene status for the sample as a diagnostic value associated with the sample. In any of the examples of this disclosure, the method further comprises generating a genomic profile for the subject based on the determination of the receptor gene status. In one or more of the examples of this disclosure, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In one or more of the examples of this disclosure, the genomic profile for the subject further comprises results from a nucleic acid sequencing-based test. In one or more of the examples of this disclosure, the method further comprises selecting an anti-cancer therapy, administering an anti-cancer therapy, or applying an anti-cancer therapy to the subject based on the generated genomic profile.

In any of the examples of this disclosure, the determination of receptor gene status for the sample is used in making suggested treatment decisions for the subject. In any of the examples of this disclosure, the determination of the receptor gene status for the sample is used in applying or administering a treatment to the subject.

In some instances, for example, systems are described that comprise one or more processors and a memory communicatively coupled to the one or more processors and configured to store instructions. The instructions, when executed by the one or more processors, cause the system to: receive, using one or more processors, training data comprising training values for a plurality of training input features associated with a receptor gene status in a plurality of training samples; train, using the one or more processors, a statistical model based on the training data; obtain, using the one or more processors, a set of input features associated with the receptor gene status, wherein obtaining the set of input features comprises omitting input features with a low prevalence and high correlation to the statistical model; receive, using one or more processors, sequence read data associated with a sample from an individual; determine, using the one or more processors, values for one or more input features associated with the receptor gene status based on the sequence read data, wherein the one or more input features to be evaluated are specified based on a sample type of the sample; input, using the one or more processors, the values for the one or more input features into the trained statistical model; and predict using the one or more processors, the receptor gene status of the individual based on an output of the trained statistical model.

In some instances, for example, non-transitory computer-readable storage mediums are described. The non-transitory computer-readable storage mediums can store one or more programs, the one or more programs comprising instructions, which when executed by one or more processors of a system, cause the system to: receive, using one or more processors, training data comprising training values for a plurality of training input features associated with a receptor gene status in a plurality of training samples; train, using the one or more processors, a statistical model based on the training data; obtain, using the one or more processors, a set of input features associated with the receptor gene status, wherein obtaining the set of input features comprises omitting input features with a low prevalence and high correlation to the statistical model; receive, using one or more processors, sequence read data associated with a sample from an individual; determine, using the one or more processors, values for one or more input features associated with the receptor gene status based on the sequence read data, wherein the one or more input features to be evaluated are specified based on a sample type of the sample; input, using the one or more processors, the values for the one or more input features into the trained statistical model; and predict using the one or more processors, the receptor gene status of the individual based on an output of the trained statistical model.

In some instances, methods are described for predicting a receptor gene status of a sample from an individual. The method can comprise receiving, using one or more processors, sequence read data associated with a sample from an individual; obtaining a set of input features associated with the receptor gene status based on an expected prevalence and correlation to the sequence read data and on a sample type associated with the type of sample; determining, using the one or more processors, values for one or more input features associated with the receptor gene status based on the sequence read data, wherein the one or more input features to be evaluated are specified based on a sample type of the sample; inputting, using the one or more processors, the values for the one or more input features into a statistical model; and predicting, using the one or more processors, the receptor gene status of the individual based on an output of the statistical model.

In any of the examples of this disclosure, the receptor gene status corresponds to an estrogen receptor status, a progesterone receptor status, an androgen receptor status, or a human epidermal growth factor receptor 2 status. In any of the examples of this disclosure, the method further comprises applying the trained machine learning model to the values for the one or more input features to obtain an output indicative of the receptor gene status. In any of the examples of tis disclosure, sequence read data derived from a liquid sample is associated with a first set of one or more input features and sequence read data derived from a solid sample is associated with a second set of one or more input features.

In any of the examples of this disclosure, the sequence read data for the individual is derived from a solid sample. In one or more examples of this disclosure, the sequence read data for the individual is derived from a biopsy sample. In one or more examples of this disclosure, the set of input features comprises genomic alteration features, complex mutational signatures, chromosomal instability features, clinicopathological features, clinical features, additional features, or a combination thereof.

In one or more examples of this disclosure, the one or more input features are associated with one or more genomic alteration features. In one or more examples of this disclosure, the one or more genomic alteration features comprise a presence of a predetermined short variant, an absence of a predetermined short variant, a copy number alteration, a rearrangement alteration, an amplification, a deletion, or a combination thereof. In one or more examples of this disclosure, the predetermined short variant comprises a point mutation, an insertion, a deletion.

In one or more examples of this disclosure, the one or more genomic alteration features comprise CCNE1, EGFR, ERBB2, ERBB2 Amplification, ESR1, FGF3, FGFR1, GATA3, MAP2K4, MYC, PIK3CA, PTEN, RB1, TP53, ZNF703, or a combination thereof. In one or more examples of this disclosure, the one or more genomic alteration features comprise BRCA1, TP53, MYC, ERBB2, ERBB2 Amplification, ERBB2 Short Variant, PIK3CA, ESR1, TBX3, CDH1, ZNF703, GATA3, FGF3, CCNE1, RB1, FGFR1, PTEN, EGFR, MAP2K4, or a combination thereof.

In one or more examples of this disclosure, the one or more genomic alteration features comprise AKT1, AKT2, AKT3, APC, AR, ARFRP1, ARIDIA, ATM, AURKA, BAP1, BCL2L1, BCL2L2, BRAF, BRCA1, BRCA2, BRD4, CBFB, CCND3, CCNE1, CDH1, CDK12, CDK4, CDK6, CDKN2A, CDKN2B, CHEK2, CREBBP, CTNNA1, DNMT3A, EGFR, EMSY, EP300, ERBB2, ERBB2 Amplification, ERBB2 Short Variant, ERBB3, ESR1, FANCA, FAS, FBXW7, FGF10, FGF12, FGF3, FGF6, FGFR1, FGFR2, FGFR4, GATA3, GNAS, HGF, IGFIR, IKBKE, IRS2, JAK2, KDM5A, KDM6A, KDR, KEL, KIT, KMT2D, KRAS, LYN, MAP2K4, MAP3K1, MCL1, MDM2, MDM4, MSH6, MUTYH, MYC, MYCL, NF1, NFKBIA, NKX2 1, NOTCH1, NOTCH2, NOTCH3,, TRK1, PALB2, PBRM1, PDCD1LG2, PDGFRA, PIK3C2B, PIK3CA, PIK3CB, PIK3R1, PRKCI, PTEN, RAF1, RB1, RET, RICTOR, ROS1, RPTOR, SETD2, SF3B1, SMAD4, SMARCA4, SOX2, SPEN, SRC, STK11, TBX3, TERC, TET2, TP53, TSC1, VEGFA, ZNF217, ZNF703, or a combination thereof.

In one or more examples of this disclosure, the one or more input features are associated with one or more complex mutational signatures. In one or more examples of this disclosure, the one or more complex mutational signatures comprise a genome-wide loss of heterozygosity (gLOH) quantification, trinucleotide signatures, insertion signatures, deletion signatures, copy number signatures, or a combination thereof.

In one or more examples of this disclosure, the one or more input features are associated with one or more chromosomal instability features. In one or more examples of this disclosure, the one or more chromosomal instability features is indicative of aneuploidy. In one or more examples of this disclosure, the one or more chromosomal instability features is indicative of chromosome gain, chromosome not gain, chromosome loss, chromosome not loss, or a combination thereof. In one or more examples of this disclosure, the one or more chromosomal instability features comprises a total aneuploidy count.

In one or more examples of this disclosure, the one or more chromosomal instability features comprise chromosome 1q loss status, chromosome 1p loss status, chromosome 3p loss status, chromosome 4p loss status, chromosome 5q loss status, chromosome 9p loss status, chromosome 10q loss status, chromosome 11q loss status, chromosome 12q loss status, chromosome 14q loss status, chromosome 15q loss status, chromosome 16p loss status, chromosome 16q loss status, chromosome 17q loss status, chromosome 19p loss status, chromosome 21q loss status, chromosome 22q loss status, chromosome 1q gain status, chromosome 2p gain status, chromosome 5p gain status, chromosome 5q gain status, chromosome 6q gain status, chromosome 7q gain status, chromosome 9p gain status, chromosome 10p gain status, chromosome 12q gain status, chromosome 16p gain status, chromosome 18p gain status, chromosome 20q gain status chromosome 21p gain status, chromosome 21q gain status.

In one or more examples of this disclosure, the one or more chromosomal instability features comprise chromosome 1p gain status, chromosome 1q gain status, chromosome2p gain status, chromosome 2q gain status, chromosome 3p gain status, chromosome 3q gain status, chromosome 4p gain status, chromosome 4q gain status, chromosome 5p gain status, chromosome 5q gain status, chromosome 6p gain status, chromosome 6q gain status, chromosome 7p gain status, chromosome 7q gain status, chromosome 8p gain status, chromosome 8q gain status, chromosome 9p gain status, chromosome 9q gain status, chromosome 10p gain status, chromosome 10q gain status, chromosome 11p gain status, chromosome 11q gain status, chromosome 12p gain status, chromosome 12q gain status, chromosome 13q gain status, chromosome 14q gain status, chromosome 15q gain status, chromosome 16p gain status, chromosome 16q gain status, chromosome 17p gain status, chromosome 17q gain status, chromosome 18p gain status, chromosome 18q gain status, chromosome 19p gain status, chromosome 19q gain status, chromosome 20p gain status, chromosome 20q gain status, chromosome 21p gain status, chromosome 21q gain status, chromosome 22q gain status, chromosome 1p loss status, chromosome 2p loss status, chromosome 2q loss status, chromosome 3p loss status, chromosome 3q loss status, chromosome 4p loss status, chromosome 4q loss status, chromosome 5p loss status, chromosome 5q loss status, chromosome 6p loss status, chromosome 6q loss status, chromosome 7p loss status, chromosome 7q loss status, chromosome 8p loss status, chromosome 8q loss status, chromosome 9p loss status, chromosome 9q loss status, chromosome 10p loss status, chromosome 10q loss status, chromosome 11p loss status, chromosome 11q loss status, chromosome 12p loss status, chromosome 12q loss status, chromosome 13q loss status, chromosome 14q loss status, chromosome 15q loss status, chromosome 16p loss status, chromosome 16q loss status, chromosome 17p loss status, chromosome 17q loss status, chromosome 18p loss status, chromosome 18q loss status, chromosome 19p loss status, chromosome 19q loss status, chromosome 20p loss status, chromosome 20q loss status, chromosome 21p loss status, chromosome 21q loss status, chromosome 22q loss status.

In one or more examples of this disclosure, the one or more input features are associated with one or more clinicopathological features. In one or more examples of this disclosure, the one or more clinicopathological features comprise an age of the individual, a sex of the individual, a disease diagnosis of the individual, a tumor status of the individual, a tumor type, an anatomical sub-classification of a tumor, or a combination thereof.

In one or more examples of this disclosure, the one or more input features are associated with one or more clinical features. In one or more examples of this disclosure, the one or more clinical features comprise an ancestry of the individual, a family history of the individual, a stage of the disease, one or more laboratory-based test results, one or more lifestyle habits, a diabetes status, an obesity status, an indication of body vitamin levels, or a combination thereof.

In one or more examples of this disclosure, wherein the one or more input features are associated with a tumor mutational burden. In one or more examples of this disclosure, the one or more input features are associated with a germline status. In one or more examples of this disclosure, the one or more input features are associated with homologous repair deficiency (HRD) signature. In one or more examples of this disclosure, the one or more input features are associated with a methylation signature, a mRNA expression level, a miRNA expression level, proteomics, cosmic mutation signatures, immunohistochemical markers, genetic predispositions, cell adhesion biomarkers, saliva based biomarkers, enzyme based biomarkers, a diagnostic image, or a combination thereof.

In one or more of the examples of this disclosure, the sequence read data for the individual is derived from a liquid sample. In one or more of the examples of this disclosure, the sequence read data for the individual is derived from a liquid biopsy sample. In one or more of the examples of this disclosure, the set of input features comprises genomic alteration features, clinicopathological features, clinical features, tumor fraction features, fragmentomic features, additional features, or a combination thereof.

In one or more of the examples of this disclosure, the one or more input features are associated with one or more genomic alteration features. In one or more of the examples of this disclosure, the one or more genomic alteration features comprise a presence of a predetermined short variant, an absence of a predetermined short variant, a rearrangement alteration, an insertion, a deletion, or a combination thereof.

In one or more of the examples of this disclosure, the one or more genomic alteration features comprise TP53, ESR1, PIK3CA, or a combination thereof. In one or more of the examples of this disclosure, the one or more genomic alteration features comprise CDH1, BRCA1, AKT1, BRCA2, CDKN2A, ERBB2, KRAS, NF1, PTEN, or a combination thereof.

In one or more of the examples of this disclosure, the one or more input features are associated with one or more clinicopathological features. In one or more of the examples of this disclosure, the one or more clinicopathological features comprise an age of the individual, a sex of the individual, a disease diagnosis of the individual, a tumor type, or a combination thereof.

In one or more of the examples of this disclosure, the one or more input features are associated with one or more clinical features. In one or more of the examples of this disclosure, the one or more clinical features comprise an ancestry of the individual, a stage of the disease, one or more laboratory-based test results, one or more lifestyle habits, a diabetes status, an obesity status, an indication of body vitamin levels, or a combination thereof.

In one or more of the examples of this disclosure, the one or more input features are associated with a tumor mutational burden. In one or more of the examples of this disclosure, the one or more input features are associated with trinucleotide signatures, insertion signatures, deletion signatures, copy number signatures, or a combination thereof. In one or more of the examples of this disclosure, the one or more input features are associated with the HRD signature.

In one or more of the examples of this disclosure, the one or more input features are associated with one or more fragmentomic features, the one or more fragmentomic features associated with a plurality of reads obtained from the sequence read data. In one or more of the examples of this disclosure, the one or more fragmentomic features comprise lengths of the plurality reads, regions associated with the plurality of reads, or a combination thereof. In one or more of the examples of this disclosure, the one or more fragmentomic features comprise an amount of a fragment having a specified length, a mean fragment length of the plurality of reads, a median fragment length of the plurality of reads, an interquartile range of fragment lengths of the plurality of reads, a distribution of fragment lengths of the plurality of reads, one or more peaks of fragment lengths for the plurality of reads, a fragment length for the plurality of reads, a start position of a fragment for the plurality of reads, and an end position of a fragment for the plurality of reads.

In one or more of the examples of this disclosure, the one or more input features are associated with an estimated tumor fraction. In one or more of the examples of this disclosure, the one or more input features are associated with a methylation signature, a mRNA expression level, a miRNA expression level, proteomics, cosmic mutation signatures, immunohistochemical markers, genetic predispositions, cell adhesion biomarkers, saliva based biomarkers, enzyme based biomarkers, a diagnostic image, or a combination thereof.

In one or more of the examples of this disclosure, the output of the trained statistical model is indicative of a receptor status. In one or more of the examples of this disclosure, the output of the trained statistical model comprises a first score indicative of a probability of a positive receptor gene status. In one or more of the examples of this disclosure, the output of the trained statistical model comprises a second score indicative of a probability of a negative receptor gene status.

In any of the examples of this disclosure, the training set of input features associated with the training values for the input features is different from the values for one or more input features input into the trained statistical model. In any of the examples of this disclosure, the training data further comprises receptor gene statuses corresponding to each of the plurality of training samples. In one or more examples of this disclosure, the method further comprises determining, using the one or more processors, weights associated with the training values for the plurality of training input features based on the training; and filtering, using the one or more processors, the plurality of training input features based on the weights; wherein filtering the plurality of training input features comprises removing low prevalence training values, highly correlated training values, or a combination thereof. In one or more examples of this disclosure, the method further comprises weighting, using the one or more processors, the training values for the one or more input features based on the weights, wherein predicting the receptor gene status is based on the weighted values for the one or more input features.

In any of the examples of this disclosure, the receptor gene status comprises a hormone receptor status. In any of the examples of this disclosure, the trained statistical model is a trained machine learning model, a part of a machine learning process, or a combination thereof. In any of the examples of this disclosure, the trained statistical model includes an artificial intelligence learning model. In any of the examples of this disclosure, the trained statistical model comprises a random forest model.

In any of the examples of this disclosure, the trained statistical model comprises at least one of an extreme gradient boosting model, a logistic regression model, an elastic net model, a ridge regression model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.

In any of the examples of this disclosure, the method further comprises: inputting, using the one or more processors, a diagnostic image taken of the individual into a second statistical model; and determining, using the one or more processors, a third score indicative of a tumor classification based on the diagnostic image, wherein predicting the receptor gene status is further based on the third score. In one or more of the examples of this disclosure, the second statistical model is at least one of a deep learning model, a convolutional neural network model, a recurrent neural network model, and an auto-encoder model.

In one or more of the examples of this disclosure, the method further comprises training the second statistical model, wherein the second statistical model is trained by: receiving, using the one or more processors, training data including a plurality of training diagnostic images; and training, using the one or more processors, the second statistical model based on the training data. In one or more of the examples of this disclosure, the diagnostic image comprises at least one of a histopathological image, a radiological image, a magnetic resonance image, an ultrasound image, an X-ray image, a bone scan, a CT scan, a PET scan, or a combination thereof.

In any of the examples of this disclosure, the method further comprises assigning, using the one or more processors, a therapy for the individual based on the predicted receptor gene status. In any of the examples of this disclosure, the method further comprises determining, using the one or more processors, a treatment decision for the individual based on the predicted receptor gene status. In any of the examples of this disclosure, the method further comprises administering, using the one or more processors, a treatment to the individual based on the predicted receptor gene status. In any of the examples of this disclosure, the method further comprises monitoring, using the one or more processors, a prognosis of the individual based on the predicted receptor gene status. In any of the examples of this disclosure, the method further comprises predicting, using the one or more processors, one or more clinical outcomes based on the predicted receptor gene status.

In some instances, methods of the present disclosure are directed to methods for diagnosing a disease, the method comprising: diagnosing that a subject has the disease based on a determination of a receptor gene status for a sample from the subject, wherein the receptor gene status is determined according to the method of any one of the methods described above.

In some instances, methods of the present disclosure are directed to methods for selecting an anti-cancer therapy, the method comprising: responsive to determining a receptor gene status for a sample from a subject, selecting an anti-cancer therapy for the subject, wherein the receptor gene status is determined according to the method of any one of the methods described above.

In some instances, methods of the present disclosure are directed to methods of treating a cancer in a subject, comprising: responsive to determining a receptor gene status for a sample from the subject, administering an effective amount of an anti-cancer therapy to the subject, wherein the receptor gene status is determined according to the method of any one of the methods described above.

In some instances, methods of the present disclosure are directed to methods for monitoring cancer progression or recurrence in a subject, the method comprising: determining a first receptor gene status in a first sample obtained from the subject at a first time point according to the method of any one of the methods described above; determining a second receptor gene status in a second sample obtained from the subject at a second time point; and comparing the first receptor gene status to the second receptor gene status, thereby monitoring the cancer progression or recurrence. In one or more of the examples of this disclosure, the second receptor gene status for the second sample is determined according to the method of any one of the methods described above.

In one or more of the examples of this disclosure, the method further comprises selecting an anti-cancer therapy for the subject in response to the cancer progression. In one or more of the examples of this disclosure, the method further comprises administering an anti-cancer therapy to the subject in response to the cancer progression. In one or more of the examples of this disclosure, the method further comprises adjusting an anti-cancer therapy for the subject in response to the cancer progression. In one or more of the examples of this disclosure, the method further comprises adjusting a dosage of the anti-cancer therapy or selecting a different anti-cancer therapy in response to the cancer progression. In one or more of the examples of this disclosure, the method further comprises administering the adjusted anti-cancer therapy to the subject.

In one or more of the examples of this disclosure the first time point is before the subject has been administered an anti-cancer therapy, and wherein the second time point is after the subject has been administered the anti-cancer therapy. In one or more of the examples of this disclosure, the subject has a cancer, is at risk of having a cancer, is being routine tested for cancer, or is suspected of having a cancer.

In one or more of the examples of this disclosure, the cancer is a solid tumor. In one or more of the examples of this disclosure, the cancer is a breast cancer. In one or more of the examples of this disclosure, the anti-cancer therapy comprises chemotherapy, radiation therapy, immunotherapy, a targeted therapy, or surgery.

In any of the examples of this disclosure, the method further comprises determining, identifying, or applying the receptor gene status for the sample as a diagnostic value associated with the sample. In any of the examples of this disclosure, the method further comprises generating a genomic profile for the subject based on the determination of the receptor gene status. In one or more of the examples of this disclosure, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In one or more of the examples of this disclosure, the genomic profile for the subject further comprises results from a nucleic acid sequencing-based test. In one or more of the examples of this disclosure, the method further comprises selecting an anti-cancer therapy, administering an anti-cancer therapy, or applying an anti-cancer therapy to the subject based on the generated genomic profile.

In any of the examples of this disclosure, the determination of receptor gene status for the sample is used in making suggested treatment decisions for the subject. In any of the examples of this disclosure, the determination of the receptor gene status for the sample is used in applying or administering a treatment to the subject.

In some instances, for example, systems are described that comprise one or more processors and a memory communicatively coupled to the one or more processors and configured to store instructions. The instructions, when executed by the one or more processors, cause the system to: receive, using one or more processors, sequence read data associated with a sample from an individual; obtain a set of input features associated with the receptor gene status based on an expected prevalence and correlation to the sequence read data and on a sample type associated with the type of sample; determine, using the one or more processors, values for one or more input features associated with the receptor gene status based on the sequence read data, wherein the one or more input features to be evaluated are specified based on a sample type of the sample; input, using the one or more processors, the values for the one or more input features into a statistical model; and predict, using the one or more processors, the receptor gene status of the individual based on an output of the statistical model.

In some instances, for example, non-transitory computer-readable storage mediums are described. The non-transitory computer-readable storage mediums can store one or more programs comprising instructions, which when executed by one or more processors of a system, cause the system to: receive, using one or more processors, sequence read data associated with a sample from an individual; obtain a set of input features associated with the receptor gene status based on an expected prevalence and correlation to the sequence read data and on a sample type associated with the type of sample; determine, using the one or more processors, values for one or more input features associated with the receptor gene status based on the sequence read data, wherein the one or more input features to be evaluated are specified based on a sample type of the sample; input, using the one or more processors, the values for the one or more input features into a statistical model; and predict, using the one or more processors, the receptor gene status of the individual based on an output of the statistical model.

In some instances, the methods described can comprise receiving, using one or more processors, sequence read data associated with a sample from an individual; obtaining a set of expression input features associated with a receptor gene status based on an expected prevalence and correlation to the sequence read data and on a sample type associated with the type of sample; determining, using the one or more processors, values for one or more expression input features associated with the receptor gene status based on the sequence read data, wherein the expression input features to be evaluated are specified based on a sample type of the sample; inputting, using the one or more processors, the values for the one or more expression input features into the statistical model; and predicting, using the one or more processors, the receptor gene status of the individual based on an output of the statistical model associated with the one or more expression input features.

In any of the examples of this disclosure, the receptor gene status corresponds to an estrogen receptor status, a progesterone receptor status, an androgen receptor status, or a human epidermal growth factor receptor 2 status. In any of the examples of this disclosure, the method further comprises applying the trained machine learning model to the values for the one or more expression input features to obtain an output indicative of the receptor gene status. In any of the examples of tis disclosure, sequence read data derived from a liquid sample is associated with a first set of one or more input features and sequence read data derived from a solid sample is associated with a second set of one or more input features.

In any of the examples of this disclosure, the sequence read data for the individual is derived from a solid sample. In one or more examples of this disclosure, the sequence read data for the individual is derived from a biopsy sample. In one or more examples of this disclosure, the set of expression input features comprises genomic alteration features, complex mutational signatures, chromosomal instability features, clinicopathological features, clinical features, additional features, or a combination thereof.

In one or more examples of this disclosure, the one or more expression input features are associated with one or more genomic alteration features. In one or more examples of this disclosure, the one or more genomic alteration features comprise a presence of a predetermined short variant, an absence of a predetermined short variant, a copy number alteration, a rearrangement alteration, an amplification, a deletion, or a combination thereof. In one or more examples of this disclosure, the predetermined short variant comprises a point mutation, an insertion, a deletion.

In one or more examples of this disclosure, the one or more genomic alteration features comprise CCNE1, EGFR, ERBB2, ERBB2 Amplification, ESR1, FGF3, FGFR1, GATA3, MAP2K4, MYC, PIK3CA, PTEN, RB1, TP53, ZNF703, or a combination thereof. In one or more examples of this disclosure, the one or more genomic alteration features comprise BRCA1, TP53, MYC, ERBB2, ERBB2 Amplification, ERBB2 Short Variant, PIK3CA, ESR1, TBX3, CDH1, ZNF703, GATA3, FGF3, CCNE1, RB1, FGFR1, PTEN, EGFR, MAP2K4, or a combination thereof.

In one or more examples of this disclosure, the one or more genomic alteration features comprise AKT1, AKT2, AKT3, APC, AR, ARFRP1, ARID1A, ATM, AURKA, BAP1, BCL2L1, BCL2L2, BRAF, BRCA1, BRCA2, BRD4, CBFB, CCND3, CCNE1, CDH1, CDK12, CDK4, CDK6, CDKN2A, CDKN2B, CHEK2, CREBBP, CTNNA1, DNMT3A, EGFR, EMSY, EP300, ERBB2, ERBB2 Amplification, ERBB2 Short Variant, ERBB3, ESR1, FANCA, FAS, FBXW7, FGF10, FGF12, FGF3, FGF6, FGFR1, FGFR2, FGFR4, GATA3, GNAS, HGF, IGF1R, IKBKE, IRS2, JAK2, KDM5A, KDM6A, KDR, KEL, KIT, KMT2D, KRAS, LYN, MAP2K4, MAP3K1, MCL1, MDM2, MDM4, MSH6, MUTYH, MYC, MYCL, NF1, NFKBIA, NKX2 1, NOTCH1, NOTCH2, NOTCH3,, TRK1, PALB2, PBRM1, PDCD1LG2, PDGFRA, PIK3C2B, PIK3CA, PIK3CB, PIK3R1, PRKCI, PTEN, RAF1, RB1, RET, RICTOR, ROS1, RPTOR, SETD2, SF3B1, SMAD4, SMARCA4, SOX2, SPEN, SRC, STK11, TBX3, TERC, TET2, TP53, TSC1, VEGFA, ZNF217, ZNF703, or a combination thereof.

In one or more examples of this disclosure, the one or more expression input features are associated with one or more complex mutational signatures. In one or more examples of this disclosure, the one or more complex mutational signatures comprise a genome-wide loss of heterozygosity (gLOH) quantification, trinucleotide signatures, insertion signatures, deletion signatures, copy number signatures, or a combination thereof.

In one or more examples of this disclosure, the one or more expression input features are associated with one or more chromosomal instability features. In one or more examples of this disclosure, the one or more chromosomal instability features is indicative of aneuploidy. In one or more examples of this disclosure, the one or more chromosomal instability features is indicative of chromosome gain, chromosome not gain, chromosome loss, chromosome not loss, or a combination thereof. In one or more examples of this disclosure, the one or more chromosomal instability features comprises a total aneuploidy count.

In one or more examples of this disclosure, the one or more chromosomal instability features comprise chromosome 1q loss status, chromosome 1p loss status, chromosome 3p loss status, chromosome 4p loss status, chromosome 5q loss status, chromosome 9p loss status, chromosome 10q loss status, chromosome 11q loss status, chromosome 12q loss status, chromosome 14q loss status, chromosome 15q loss status, chromosome 16p loss status, chromosome 16q loss status, chromosome 17q loss status, chromosome 19p loss status, chromosome 21q loss status, chromosome 22q loss status, chromosome 1q gain status, chromosome 2p gain status, chromosome 5p gain status, chromosome 5q gain status, chromosome 6q gain status, chromosome 7q gain status, chromosome 9p gain status, chromosome 10p gain status, chromosome 12q gain status, chromosome 16p gain status, chromosome 18p gain status, chromosome 20q gain status chromosome 21p gain status, chromosome 21q gain status.

In one or more examples of this disclosure, the one or more chromosomal instability features comprise chromosome 1p gain status, chromosome 1q gain status, chromosome2p gain status, chromosome 2q gain status, chromosome 3p gain status, chromosome 3q gain status, chromosome 4p gain status, chromosome 4q gain status, chromosome 5p gain status, chromosome 5q gain status, chromosome 6p gain status, chromosome 6q gain status, chromosome 7p gain status, chromosome 7q gain status, chromosome 8p gain status, chromosome 8q gain status, chromosome 9p gain status, chromosome 9q gain status, chromosome 10p gain status, chromosome 10q gain status, chromosome 11p gain status, chromosome 11q gain status, chromosome 12p gain status, chromosome 12q gain status, chromosome 13q gain status, chromosome 14q gain status, chromosome 15q gain status, chromosome 16p gain status, chromosome 16q gain status, chromosome 17p gain status, chromosome 17q gain status, chromosome 18p gain status, chromosome 18q gain status, chromosome 19p gain status, chromosome 19q gain status, chromosome 20p gain status, chromosome 20q gain status, chromosome 21p gain status, chromosome 21q gain status, chromosome 22q gain status, chromosome 1p loss status, chromosome 2p loss status, chromosome 2q loss status, chromosome 3p loss status, chromosome 3q loss status, chromosome 4p loss status, chromosome 4q loss status, chromosome 5p loss status, chromosome 5q loss status, chromosome 6p loss status, chromosome 6q loss status, chromosome 7p loss status, chromosome 7q loss status, chromosome 8p loss status, chromosome 8q loss status, chromosome 9p loss status, chromosome 9q loss status, chromosome 10p loss status, chromosome 10q loss status, chromosome 11p loss status, chromosome 11q loss status, chromosome 12p loss status, chromosome 12q loss status, chromosome 13q loss status, chromosome 14q loss status, chromosome 15q loss status, chromosome 16p loss status, chromosome 16q loss status, chromosome 17p loss status, chromosome 17q loss status, chromosome 18p loss status, chromosome 18q loss status, chromosome 19p loss status, chromosome 19q loss status, chromosome 20p loss status, chromosome 20q loss status, chromosome 21p loss status, chromosome 21q loss status, chromosome 22q loss status.

In one or more examples of this disclosure, the one or more expression input features are associated with one or more clinicopathological features. In one or more examples of this disclosure, the one or more clinicopathological features comprise an age of the individual, a sex of the individual, a disease diagnosis of the individual, a tumor status of the individual, a tumor type, an anatomical sub-classification of a tumor, or a combination thereof.

In one or more examples of this disclosure, the one or more expression input features are associated with one or more clinical features. In one or more examples of this disclosure, the one or more clinical features comprise an ancestry of the individual, a family history of the individual, a stage of the disease, one or more laboratory-based test results, one or more lifestyle habits, a diabetes status, an obesity status, an indication of body vitamin levels, or a combination thereof.

In one or more examples of this disclosure, wherein the one or more expression input features are associated with a tumor mutational burden. In one or more examples of this disclosure, the one or more expression input features are associated with a germline status. In one or more examples of this disclosure, the one or more expression input features are associated with homologous repair deficiency (HRD) signature. In one or more examples of this disclosure, the one or more expression input features are associated with a methylation signature, a mRNA expression level, a miRNA expression level, proteomics, cosmic mutation signatures, immunohistochemical markers, genetic predispositions, cell adhesion biomarkers, saliva based biomarkers, enzyme based biomarkers, a diagnostic image, or a combination thereof.

In one or more of the examples of this disclosure, the sequence read data for the individual is derived from a liquid sample. In one or more of the examples of this disclosure, the sequence read data for the individual is derived from a liquid biopsy sample. In one or more of the examples of this disclosure, the set of expression input features comprises genomic alteration features, clinicopathological features, clinical features, tumor fraction features, fragmentomic features, additional features, or a combination thereof.

In one or more of the examples of this disclosure, the one or more expression input features are associated with one or more genomic alteration features. In one or more of the examples of this disclosure, the one or more genomic alteration features comprise a presence of a predetermined short variant, an absence of a predetermined short variant, a rearrangement alteration, an insertion, a deletion, or a combination thereof.

In one or more of the examples of this disclosure, the one or more genomic alteration features comprise TP53, ESR1, PIK3CA, or a combination thereof. In one or more of the examples of this disclosure, the one or more genomic alteration features comprise CDH1, BRCA1, AKT1, BRCA2, CDKN2A, ERBB2, KRAS, NF1, PTEN, or a combination thereof.

In one or more of the examples of this disclosure, the one or more expression input features are associated with one or more clinicopathological features. In one or more of the examples of this disclosure, the one or more clinicopathological features comprise an age of the individual, a sex of the individual, a disease diagnosis of the individual, a tumor type, or a combination thereof.

In one or more of the examples of this disclosure, the one or more expression input features are associated with one or more clinical features. In one or more of the examples of this disclosure, the one or more clinical features comprise an ancestry of the individual, a stage of the disease, one or more laboratory-based test results, one or more lifestyle habits, a diabetes status, an obesity status, an indication of body vitamin levels, or a combination thereof.

In one or more of the examples of this disclosure, the one or more expression input features are associated with a tumor mutational burden. In one or more of the examples of this disclosure, the one or more expression input features are associated with trinucleotide signatures, insertion signatures, deletion signatures, copy number signatures, or a combination thereof. In one or more of the examples of this disclosure, the one or more expression input features are associated with the HRD signature.

In one or more of the examples of this disclosure, the one or more expression input features are associated with one or more fragmentomic features, the one or more fragmentomic features associated with a plurality of reads obtained from the sequence read data. In one or more of the examples of this disclosure, the one or more fragmentomic features comprise lengths of the plurality reads, regions associated with the plurality of reads, or a combination thereof. In one or more of the examples of this disclosure, the one or more fragmentomic features comprise an amount of a fragment having a specified length, a mean fragment length of the plurality of reads, a median fragment length of the plurality of reads, an interquartile range of fragment lengths of the plurality of reads, a distribution of fragment lengths of the plurality of reads, one or more peaks of fragment lengths for the plurality of reads, a fragment length for the plurality of reads, a start position of a fragment for the plurality of reads, and an end position of a fragment for the plurality of reads.

In one or more of the examples of this disclosure, the one or more expression input features are associated with an estimated tumor fraction. In one or more of the examples of this disclosure, the one or more expression input features are associated with a methylation signature, an mRNA expression level, a miRNA expression level, proteomics, cosmic mutation signatures, immunohistochemical markers, genetic predispositions, cell adhesion biomarkers, saliva based biomarkers, enzyme based biomarkers, a diagnostic image, or a combination thereof.

In one or more of the examples of this disclosure, the output of the trained statistical model is indicative of a receptor status. In one or more of the examples of this disclosure, the output of the trained statistical model comprises a first score indicative of a probability of a positive receptor gene status. In one or more of the examples of this disclosure, the output of the trained statistical model comprises a second score indicative of a probability of a negative receptor gene status.

In any of the examples of this disclosure, the training set of expression input features associated with the training values for the expression input features is different from the values for one or more expression input features input into the trained statistical model. In any of the examples of this disclosure, the training data further comprises receptor gene statuses corresponding to each of the plurality of training samples. In one or more examples of this disclosure, the method further comprises determining, using the one or more processors, weights associated with the training values for the plurality of training expression input features based on the training; and filtering, using the one or more processors, the plurality of training expression input features based on the weights; wherein filtering the plurality of training expression input features comprises removing low prevalence training values, highly correlated training values, or a combination thereof. In one or more examples of this disclosure, the method further comprises weighting, using the one or more processors, the training values for the one or more expression input features based on the weights, wherein predicting the receptor gene status is based on the weighted values for the one or more expression input features.

In any of the examples of this disclosure, the receptor gene status comprises a hormone receptor status. In any of the examples of this disclosure, the trained statistical model is a trained machine learning model, a part of a machine learning process, or a combination thereof. In any of the examples of this disclosure, the trained statistical model includes an artificial intelligence learning model. In any of the examples of this disclosure, the trained statistical model comprises a random forest model.

In any of the examples of this disclosure, the trained statistical model comprises at least one of an extreme gradient boosting model, a logistic regression model, an elastic net model, a ridge regression model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.

In any of the examples of this disclosure, the method further comprises: inputting, using the one or more processors, a diagnostic image taken of the individual into a second statistical model; and determining, using the one or more processors, a third score indicative of a tumor classification based on the diagnostic image, wherein predicting the receptor gene status is further based on the third score. In one or more of the examples of this disclosure, the second statistical model is at least one of a deep learning model, a convolutional neural network model, a recurrent neural network model, and an auto-encoder model.

In one or more of the examples of this disclosure, the method further comprises training the second statistical model, wherein the second statistical model is trained by: receiving, using the one or more processors, training data including a plurality of training diagnostic images; and training, using the one or more processors, the second statistical model based on the training data. In one or more of the examples of this disclosure, the diagnostic image comprises at least one of a histopathological image, a radiological image, a magnetic resonance image, an ultrasound image, an X-ray image, a bone scan, a CT scan, a PET scan, or a combination thereof.

In any of the examples of this disclosure, the method further comprises assigning, using the one or more processors, a therapy for the individual based on the predicted receptor gene status. In any of the examples of this disclosure, the method further comprises determining, using the one or more processors, a treatment decision for the individual based on the predicted receptor gene status. In any of the examples of this disclosure, the method further comprises administering, using the one or more processors, a treatment to the individual based on the predicted receptor gene status. In any of the examples of this disclosure, the method further comprises monitoring, using the one or more processors, a prognosis of the individual based on the predicted receptor gene status. In any of the examples of this disclosure, the method further comprises predicting, using the one or more processors, one or more clinical outcomes based on the predicted receptor gene status.

In some instances, methods of the present disclosure are directed to methods for diagnosing a disease, the method comprising: diagnosing that a subject has the disease based on a determination of a receptor gene status for a sample from the subject, wherein the receptor gene status is determined according to the method of any one of the methods described above.

In some instances, methods of the present disclosure are directed to methods for selecting an anti-cancer therapy, the method comprising: responsive to determining a receptor gene status for a sample from a subject, selecting an anti-cancer therapy for the subject, wherein the receptor gene status is determined according to the method of any one of the methods described above.

In some instances, methods of the present disclosure are directed to methods of treating a cancer in a subject, comprising: responsive to determining a receptor gene status for a sample from the subject, administering an effective amount of an anti-cancer therapy to the subject, wherein the receptor gene status is determined according to the method of any one of the methods described above.

In some instances, methods of the present disclosure are directed to methods for monitoring cancer progression or recurrence in a subject, the method comprising: determining a first receptor gene status in a first sample obtained from the subject at a first time point according to the method of any one of the methods described above; determining a second receptor gene status in a second sample obtained from the subject at a second time point; and comparing the first receptor gene status to the second receptor gene status, thereby monitoring the cancer progression or recurrence. In one or more of the examples of this disclosure, the second receptor gene status for the second sample is determined according to the method of any one of the methods described above.

In one or more of the examples of this disclosure, the method further comprises selecting an anti-cancer therapy for the subject in response to the cancer progression. In one or more of the examples of this disclosure, the method further comprises administering an anti-cancer therapy to the subject in response to the cancer progression. In one or more of the examples of this disclosure, the method further comprises adjusting an anti-cancer therapy for the subject in response to the cancer progression. In one or more of the examples of this disclosure, the method further comprises adjusting a dosage of the anti-cancer therapy or selecting a different anti-cancer therapy in response to the cancer progression. In one or more of the examples of this disclosure, the method further comprises administering the adjusted anti-cancer therapy to the subject.

In one or more of the examples of this disclosure the first time point is before the subject has been administered an anti-cancer therapy, and wherein the second time point is after the subject has been administered the anti-cancer therapy. In one or more of the examples of this disclosure, the subject has a cancer, is at risk of having a cancer, is being routine tested for cancer, or is suspected of having a cancer.

In one or more of the examples of this disclosure, the cancer is a solid tumor. In one or more of the examples of this disclosure, the cancer is a breast cancer. In one or more of the examples of this disclosure, the anti-cancer therapy comprises chemotherapy, radiation therapy, immunotherapy, a targeted therapy, or surgery.

In any of the examples of this disclosure, the method further comprises determining, identifying, or applying the receptor gene status for the sample as a diagnostic value associated with the sample. In any of the examples of this disclosure, the method further comprises generating a genomic profile for the subject based on the determination of the receptor gene status. In one or more of the examples of this disclosure, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In one or more of the examples of this disclosure, the genomic profile for the subject further comprises results from a nucleic acid sequencing-based test. In one or more of the examples of this disclosure, the method further comprises selecting an anti-cancer therapy, administering an anti-cancer therapy, or applying an anti-cancer therapy to the subject based on the generated genomic profile.

In any of the examples of this disclosure, the determination of receptor gene status for the sample is used in making suggested treatment decisions for the subject. In any of the examples of this disclosure, the determination of the receptor gene status for the sample is used in applying or administering a treatment to the subject.

In some instances, for example, systems are described that comprise one or more processors and a memory communicatively coupled to the one or more processors and configured to store instructions. The instructions, when executed by the one or more processors, cause the system to: receive, using one or more processors, sequence read data associated with a sample from an individual; obtain a set of expression input features associated with a receptor gene status based on an expected prevalence and correlation to the sequence read data and on a sample type associated with the type of sample; determine, using the one or more processors, values for one or more expression input features associated with the receptor gene status based on the sequence read data, wherein the expression input features to be evaluated are specified based on a sample type of the sample; input, using the one or more processors, the values for the one or more expression input features into the statistical model; and predict, using the one or more processors, the receptor gene status of the individual based on an output of the statistical model associated with the one or more expression input features.

In some instances, for example, non-transitory computer-readable storage mediums are described. The non-transitory computer-readable storage mediums can store one or more programs, comprising instructions, which when executed by one or more processors of a system, cause the system to: receive, using one or more processors, sequence read data associated with a sample from an individual; obtain a set of expression input features associated with a receptor gene status based on an expected prevalence and correlation to the sequence read data and on a sample type associated with the type of sample; determine, using the one or more processors, values for one or more expression input features associated with the receptor gene status based on the sequence read data, wherein the expression input features to be evaluated are specified based on a sample type of the sample; input, using the one or more processors, the values for the one or more expression input features into the statistical model; and predict, using the one or more processors, the receptor gene status of the individual based on an output of the statistical model associated with the one or more expression input features.

In some instances, the methods described can comprise receiving, using one or more processors, training data comprising values for a plurality of training input features associated with a receptor gene status in a plurality of training samples; training, using the one or more processors, a statistical model based on the training data, wherein the trained statistical model is configured to predict a receptor gene status of an individual sample; determining, using the one or more processors, weights associated with the training values for the plurality of training input features based on the training; filtering, using the one or more processors, the one or more training input features based on the weights; determining a set of input features associated with the receptor gene status based on the filtered training input features and a sample type of a sample from an individual, wherein filtering the one or more training input features comprises removing training input features associated with low prevalence training values and highly correlated training values, or a combination thereof; and obtaining a trained statistical model configured to receive a set of input feature based on a sample from an individual to output a prediction of a receptor gene status of the sample.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of an exemplary process for predicting a receptor status of a sample from an individual, according to embodiments of the present disclosure.

FIG. 2A provides a non-limiting example of input features for a statistical model according to embodiments of the present disclosure.

FIG. 2B provides a non-limiting example of input features for a statistical model according to embodiments of the present disclosure.

FIG. 2C provides a non-limiting example of input features for a statistical model according to embodiments of the present disclosure.

FIG. 2D provides a non-limiting example of input features for a statistical model according to embodiments of the present disclosure.

FIG. 2E provides a non-limiting example of input features for a statistical model according to embodiments of the present disclosure.

FIG. 2F provides a non-limiting example of input features for a statistical model according to embodiments of the present disclosure.

FIG. 2G provides a non-limiting example of input features for a statistical model according to embodiments of the present disclosure.

FIG. 2H provides a non-limiting example of input features for a statistical model according to embodiments of the present disclosure.

FIG. 2I provides a non-limiting example of input features for a statistical model according to embodiments of the present disclosure.

FIG. 2J provides a non-limiting example of input features for a statistical model according to embodiments of the present disclosure.

FIG. 2K provides a non-limiting example of input features for a statistical model according to embodiments of the present disclosure.

FIG. 2L provides a non-limiting example of input features for a statistical model according to embodiments of the present disclosure.

FIG. 3A provides a non-limiting example of input features for a statistical model according to embodiments of the present disclosure.

FIG. 3B provides a non-limiting example of input features for a statistical model according to embodiments of the present disclosure.

FIG. 3C provides a non-limiting example of input features for a statistical model according to embodiments of the present disclosure.

FIG. 3D provides a non-limiting example of input features for a statistical model according to embodiments of the present disclosure.

FIG. 3E provides a non-limiting example of input features for a statistical model according to embodiments of the present disclosure.

FIG. 3F provides a non-limiting example of input features for a statistical model according to embodiments of the present disclosure.

FIG. 3G provides a non-limiting example of input features for a statistical model according to embodiments of the present disclosure.

FIG. 3H provides a non-limiting example of input features for a statistical model according to embodiments of the present disclosure.

FIG. 3I provides a non-limiting example of input features for a statistical model according to embodiments of the present disclosure.

FIG. 4 provides a non-limiting example of using a statistical model to predict receptor status according to embodiments of the present disclosure.

FIG. 5 provides a non-limiting example of an exemplary process for predicting a receptor status of a sample from an individual, according to embodiments of the present disclosure.

FIG. 6 provides a non-limiting example of a process for training a statistical model predicting a receptor status of a sample from an individual, according to embodiments of the present disclosure FIG. 7 depicts an exemplary computing device or system, according to embodiments of the present disclosure.

FIG. 8 depicts an exemplary computer system or computer network, according to embodiments of the present disclosure.

FIG. 9 depicts an exemplary process for training a statistical model, according to embodiments of the present disclosure.

FIG. 10A provides a non-limiting example of cross validation metrics of an exemplary model using a training dataset, according to embodiments of the present disclosure.

FIG. 10B provides a non-limiting example of performance metrics of an exemplary model using a test dataset, according to embodiments of the present disclosure.

FIG. 10C provides a non-limiting example of performance metrics of an exemplary model using a validation dataset, according to embodiments of the present disclosure.

FIG. 10D provides a non-limiting example of a plot illustrating relative feature importance of input features for an exemplary model, according to embodiments of the present disclosure.

FIG. 11A provides a non-limiting example of cross validation metrics of an exemplary model using a training dataset, according to embodiments of the present disclosure.

FIG. 11B provides a non-limiting example of performance metrics of an exemplary model using a test dataset, according to embodiments of the present disclosure.

FIG. 11C provides a non-limiting example of performance metrics of an exemplary model using a validation dataset, according to embodiments of the present disclosure.

FIG. 11D provides a non-limiting example of performance metrics of an exemplary model using a validation dataset, according to embodiments of the present disclosure.

FIG. 11E provides a non-limiting example of a plot illustrating relative feature importance of input features for an exemplary model, according to embodiments of the present disclosure.

### DETAILED DESCRIPTION

Methods and systems for determining a diagnostic gene status or receptor status in a sample from an individual are described. In some instances, the systems and methods described herein may be used, for example, to determine the receptor status of an estrogen receptor (ER), a progesterone receptor (PR), an androgen receptor, or a human epidermal growth factor receptor 2 (HER2) in a sample from an individual. Determining the receptor status of a patient can be useful for informing the prognosis and treatment options for the patient. For example, breast cancer cells that express estrogen or progesterone hormone receptors (e.g., on the cell surface or inside the cell) can be stimulated and subsequently grow and propagate in the presence of the estrogen and progesterone hormones, respectively. About 70%-80% of breast cancer patients have hormone receptor positivity (ER positive status and/or PR positive status). Hormone receptor positive individuals may respond well to treatment with hormone therapy in both the adjuvant and the neoadjuvant setting. For example, hormone therapies may can be used to block the interaction between the hormone and a respective receptor (e.g., by blocking the interaction between estrogen and estrogen receptors) and/or to reduce hormone levels (e.g., by reducing estrogen levels).

Accordingly, determining the diagnostic gene status or receptor status of breast cancer patients (e.g., the estrogen receptor (ER) status and progesterone receptor (PR) status, the human epidermal growth factor receptor 2 (HER2) status, androgen status, or any combination thereof) can inform the prognosis and treatment options for patients. Generally, the receptor status of breast cancer patients can be determined using an immunohistochemistry (IHC) test based on a tissue biopsy. The IHC test can provide a semi-quantitative measurement of ER/PR positive tumor nuclei in stained histologic tissue sections. However, obtaining tissue samples can be an invasive and painful process. Moreover, in some situations, the biopsy site may be inaccessible such that it is not possible to obtain an adequate tissue sample. Accordingly, there is a need to provide a system to determine a receptor status that can be used for both solid and liquid samples.

In some instances, for example, methods are described that comprise providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of reads that represent the captured nucleic acid molecules; receiving, at one or more processors, sequence read data for the plurality of reads; receiving, using one or more processors, training data comprising training values for a plurality of training input features associated with a receptor gene status in a plurality of training samples; training, using the one or more processors, a statistical model based on the training data; obtaining, using the one or more processors, a set of input features associated with the receptor gene status, wherein obtaining the set of input features comprises omitting input features with a low prevalence and high correlation to the statistical model; determining, using the one or more processors, values for one or more input features associated with the receptor gene status based on the sequence read data, wherein the one or more input features to be evaluated are specified based on a sample type of the sample; inputting, using the one or more processors, the values for the one or more input features into the trained statistical model; and predicting, using the one or more processors, the receptor gene status of the subject based on an output of the trained statistical model.

In some instances, for example, methods are described that comprise receiving, using one or more processors, training data comprising training values for a plurality of training input features associated with a receptor gene status in a plurality of training samples; training, using the one or more processors, a statistical model based on the training data; obtaining, using the one or more processors, a set of input features associated with the receptor gene status and a sample type, wherein obtaining the set of input features comprises omitting input features with a low prevalence and high correlation to the statistical model; receiving, using one or more processors, sequence read data associated with a sample from an individual; determining, using the one or more processors, values for one or more input features associated with the receptor gene status based on the sequence read data, wherein the one or more input features to be evaluated are specified based on a sample type of the sample; inputting, using the one or more processors, the values for the one or more input features into the trained statistical model; and predicting, using the one or more processors, the receptor gene status of the individual based on an output of the trained statistical model.

In some instances, for example, systems are described that comprise one or more processors and a memory communicatively coupled to the one or more processors and configured to store instructions. The instructions, when executed by the one or more processors, cause the system to: receive, using one or more processors, training data comprising training values for a plurality of training input features associated with a receptor gene status in a plurality of training samples; train, using the one or more processors, a statistical model based on the training data; obtain, using the one or more processors, a set of input features associated with the receptor gene status, wherein obtaining the set of input features comprises omitting input features with a low prevalence and high correlation to the statistical model; receive, using one or more processors, sequence read data associated with a sample from an individual; determine, using the one or more processors, values for one or more input features associated with the receptor gene status based on the sequence read data, wherein the one or more input features to be evaluated are specified based on a sample type of the sample; input, using the one or more processors, the values for the one or more input features into the trained statistical model; and predict using the one or more processors, the receptor gene status of the individual based on an output of the trained statistical model.

In some instances, for example, non-transitory computer-readable storage mediums are described. The non-transitory computer-readable storage mediums can store one or more programs, the one or more programs comprising instructions, which when executed by one or more processors of a system, cause the system to: receive, using one or more processors, training data comprising training values for a plurality of training input features associated with a receptor gene status in a plurality of training samples; train, using the one or more processors, a statistical model based on the training data; obtain, using the one or more processors, a set of input features associated with the receptor gene status, wherein obtaining the set of input features comprises omitting input features with a low prevalence and high correlation to the statistical model; receive, using one or more processors, sequence read data associated with a sample from an individual; determine, using the one or more processors, values for one or more input features associated with the receptor gene status based on the sequence read data, wherein the one or more input features to be evaluated are specified based on a sample type of the sample; input, using the one or more processors, the values for the one or more input features into the trained statistical model; and predict using the one or more processors, the receptor gene status of the individual based on an output of the trained statistical model.

In some instances, methods are described for predicting a receptor gene status of a sample from an individual. The method can comprise receiving, using one or more processors, sequence read data associated with a sample from an individual; obtaining a set of input features associated with the receptor gene status based on an expected prevalence and correlation to the sequence read data and on a sample type associated with the type of sample; determining, using the one or more processors, values for one or more input features associated with the receptor gene status based on the sequence read data, wherein the one or more input features to be evaluated are specified based on a sample type of the sample; inputting, using the one or more processors, the values for the one or more input features into a statistical model; and predicting, using the one or more processors, the receptor gene status of the individual based on an output of the statistical model.

In some instances, for example, systems are described that comprise one or more processors and a memory communicatively coupled to the one or more processors and configured to store instructions. The instructions, when executed by the one or more processors, cause the system to: receive, using one or more processors, sequence read data associated with a sample from an individual; obtain a set of input features associated with the receptor gene status based on an expected prevalence and correlation to the sequence read data and on a sample type associated with the type of sample; determine, using the one or more processors, values for one or more input features associated with the receptor gene status based on the sequence read data, wherein the one or more input features to be evaluated are specified based on a sample type of the sample; input, using the one or more processors, the values for the one or more input features into a statistical model; and predict, using the one or more processors, the receptor gene status of the individual based on an output of the statistical model.

In some instances, for example, non-transitory computer-readable storage mediums are described. The non-transitory computer-readable storage mediums can store one or more programs comprising instructions, which when executed by one or more processors of a system, cause the system to: receive, using one or more processors, sequence read data associated with a sample from an individual; obtain a set of input features associated with the receptor gene status based on an expected prevalence and correlation to the sequence read data and on a sample type associated with the type of sample; determine, using the one or more processors, values for one or more input features associated with the receptor gene status based on the sequence read data, wherein the one or more input features to be evaluated are specified based on a sample type of the sample; input, using the one or more processors, the values for the one or more input features into a statistical model; and predict, using the one or more processors, the receptor gene status of the individual based on an output of the statistical model.

In some instances, the methods described can comprise receiving, using one or more processors, sequence read data associated with a sample from an individual; obtaining a set of expression input features associated with a receptor gene status based on an expected prevalence and correlation to the sequence read data and on a sample type associated with the type of sample; determining, using the one or more processors, values for one or more expression input features associated with the receptor gene status based on the sequence read data, wherein the expression input features to be evaluated are specified based on a sample type of the sample; inputting, using the one or more processors, the values for the one or more expression input features into the statistical model; and predicting, using the one or more processors, the receptor gene status of the individual based on an output of the statistical model associated with the one or more expression input features.

In some instances, for example, systems are described that comprise one or more processors and a memory communicatively coupled to the one or more processors and configured to store instructions. The instructions, when executed by the one or more processors, cause the system to: receive, using one or more processors, sequence read data associated with a sample from an individual; obtain a set of expression input features associated with a receptor gene status based on an expected prevalence and correlation to the sequence read data and on a sample type associated with the type of sample; determine, using the one or more processors, values for one or more expression input features associated with the receptor gene status based on the sequence read data, wherein the expression input features to be evaluated are specified based on a sample type of the sample; input, using the one or more processors, the values for the one or more expression input features into the statistical model; and predict, using the one or more processors, the receptor gene status of the individual based on an output of the statistical model associated with the one or more expression input features.

In some instances, for example, non-transitory computer-readable storage mediums are described. The non-transitory computer-readable storage mediums can store one or more programs, comprising instructions, which when executed by one or more processors of a system, cause the system to: receive, using one or more processors, sequence read data associated with a sample from an individual; obtain a set of expression input features associated with a receptor gene status based on an expected prevalence and correlation to the sequence read data and on a sample type associated with the type of sample; determine, using the one or more processors, values for one or more expression input features associated with the receptor gene status based on the sequence read data, wherein the expression input features to be evaluated are specified based on a sample type of the sample; input, using the one or more processors, the values for the one or more expression input features into the statistical model; and predict, using the one or more processors, the receptor gene status of the individual based on an output of the statistical model associated with the one or more expression input features.

In some instances, the methods described can comprise receiving, using one or more processors, training data comprising values for a plurality of training input features associated with a receptor gene status in a plurality of training samples; training, using the one or more processors, a statistical model based on the training data, wherein the trained statistical model is configured to predict a receptor gene status of an individual sample; determining, using the one or more processors, weights associated with the training values for the plurality of training input features based on the training; filtering, using the one or more processors, the one or more training input features based on the weights; determining a set of input features associated with the receptor gene status based on the filtered training input features and a sample type of a sample from an individual, wherein filtering the one or more training input features comprises removing training input features associated with low prevalence training values and highly correlated training values, or a combination thereof; and obtaining a trained statistical model configured to receive a set of input feature based on a sample from an individual to output a prediction of a receptor gene status of the sample.

The disclosed methods and systems can be used to determine a diagnostic gene status or receptor status of an individual based on a sample, e.g., a liquid or solid sample. Embodiments of the present disclosure can further be used to inform treatment decisions to improve the outcomes for individuals.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the terms “diagnostic gene status” and “receptor status” may refer to a status of a receptor corresponding to a gene or hormone that has a known pathogenic or likely pathogenic effect.

The section headings used herein are for organizational purposes only and are not to be construed as limiting the subject matter described.

### Methods for Determining a Hormone Receptor Status

The diagnostic gene status of a patient can be useful for informing the prognosis and treatment options for patients. For individuals with cancer cells (e.g., breast cancer) that express hormone receptors (e.g., estrogen receptors (ER), progesterone receptors (PR), androgen receptors) or gene receptors (e.g., human epidermal growth factor receptors 2 (HER2)) prescribing receptor-targeted therapies can be an effective way to block the interaction between the hormone or gene and a respective receptor (e.g., by blocking the interaction between estrogen and estrogen receptors) and/or to reduce the hormone levels (e.g., by reducing the estrogen levels near the estrogen receptors) to reduce stimulation and growth of the cancer cells. In some instances when tumors are associated with more than one receptor gene status, therapies may be based on one or more of the receptor gene statuses. For example, tumors that are ER positive and HER2 positive may be treated as HER2 tumors.

One or more embodiments of the present disclosure provide systems and methods for determining a hormone receptor status of an individual based on a sample from the individual.

In one or more examples, the diagnostic gene status may include, but is not limited to, an estrogen receptor (ER), a progesterone receptor (PR), an androgen receptor, or a human epidermal growth factor receptor 2 (HER2). One or more embodiments of the present disclosure may be used to determine the receptor status of a tumor in an individual based on a solid biopsy sample. One or more embodiments of the present disclosure may be used to determine the receptor status of a tumor in an individual based on a liquid biopsy sample.

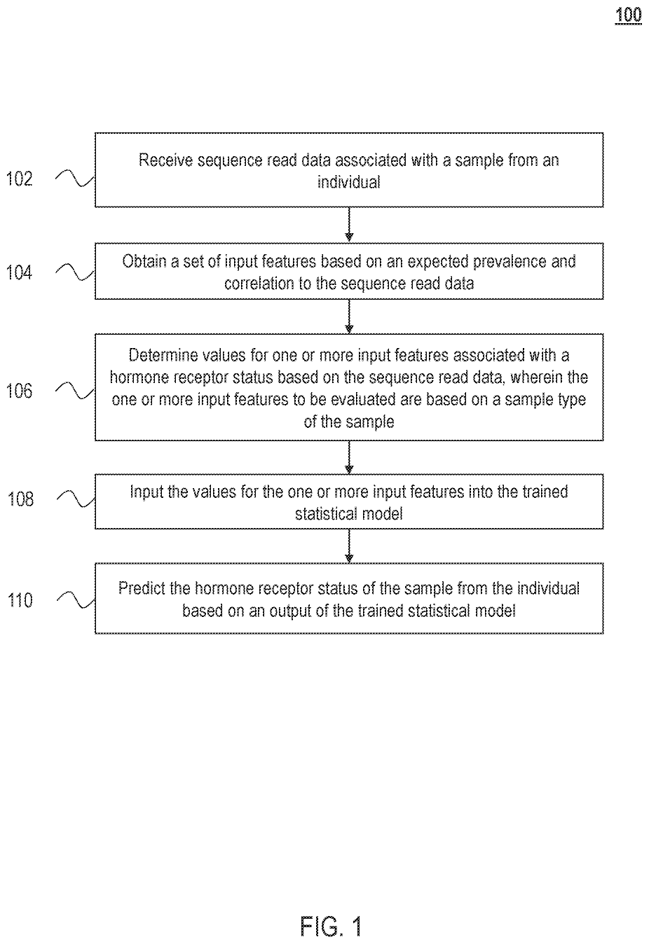

FIG. 1 provides a non-limiting example of a process 100 for determining a receptor status of a sample from an individual. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, the system can receive sequence read data associated with a sample from an individual. In some instances, the sequence read data may be derived from single region sequencing (e.g., sequencing of a single tissue biopsy sample collected from the patient tumor). In some instances, the genomic data comprising sequence read data may be derived from multi-region sequencing (e.g., sequencing of multiple tissue biopsy samples collected from the patient tumor). In some instances, the genomic data comprising sequence read data may be derived from single cell sequencing data as opposed to bulk tumor sequencing. In some instances, the genomic data comprising sequence read data may be derived from sequencing the circulating tumor DNA in a liquid biopsy sample.

In some instances, the genomic data comprising sequence read data may be derived from targeted sequencing, e.g., targeted exome sequencing. In some instances, the genomic data comprising sequence read data may be derived from, e.g., whole genome or whole exome sequencing, as opposed to targeted exome sequencing to increase the number of genomic features (e.g., the number of short variants) detected. In one or more examples, the sequence read data may be received by the system as a BAM file.

In one or more examples, the sequence read data may be indicative of a presence or absence of one or more short variants (SVs) in a patient sample. In one or more examples, the sequence read data may also be indicative of the presence or absence of other genomic features in addition to short variants, such as copy number alterations, rearrangements, insertions, deletions, fusions, chromosomal aneuploidy, whole genome doubling, Catalogue of Somatic Mutations in Cancer (COSMIC) mutational signatures, or any combination thereof.

At step 104 in FIG. 1, the system can determine a set of input features associated with a receptor status based on a sample type associated with the type of sample and based on an expected prevalence and correlation of the features to the sequence read data. For example, input features that are expected to have a low prevalence and high correlation to the sequence read data may be omitted. In one or more examples, the set of input features may differ based on the type of sample, e.g., liquid sample or solid sample. For example, the input features for a model to predict a receptor status based on a solid tissue sample may be associated with a first set of features, while the input features for a model to predict a receptor status based on a liquid sample may be associated with a second set of features. In one or more examples, one or more features of the first set of features may overlap with one or more features of the second set of features. In one or more examples, one or more features of the first set of features may differ from one or more features of the second set of features.

In one or more examples, the input features expected to have a low prevalence and be highly correlated with the sequence read data may be omitted from the set of input features. By omitting low prevalence and highly correlated features, the system may improve the reliability of the model. For example, an input feature that is found in less than one percent of tumors may not provide valuable information to predict a receptor status and may potentially skew a receptor status prediction based on such features.

In one or more examples, input features with a prevalence of less than a predetermined prevalence threshold (e.g., 1%) may be determined to have a low prevalence. In one or more examples, the predetermined prevalence threshold may be in a range of 0.1%-5%. In one or more examples, input features with a correlation greater than a predetermined correlation threshold (e.g., 90%) may be determined to be highly correlated. In one or more examples, the predetermined correlation threshold may be in a range of approximately 50%-100%, or approximately 75%-100%.

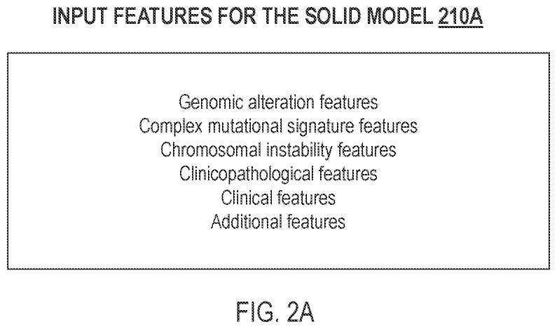

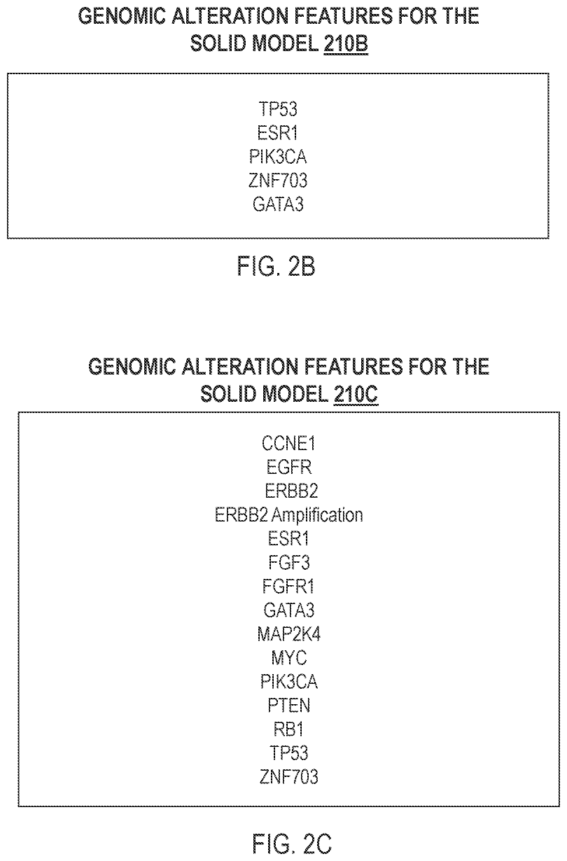

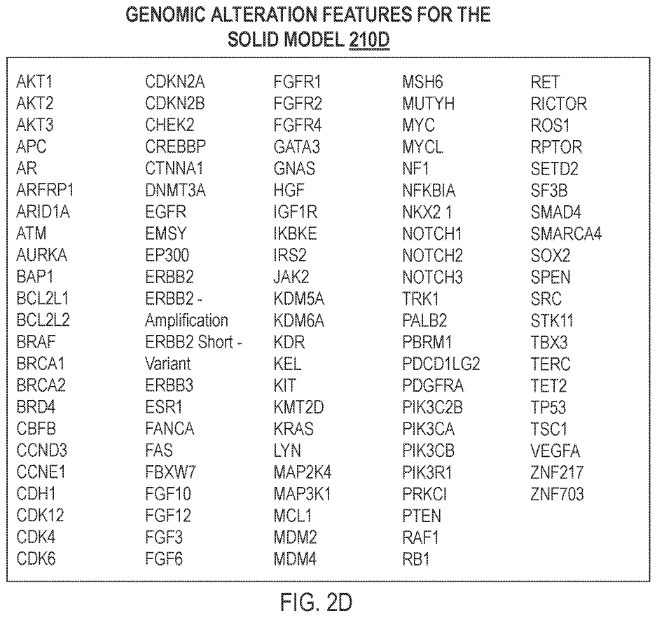

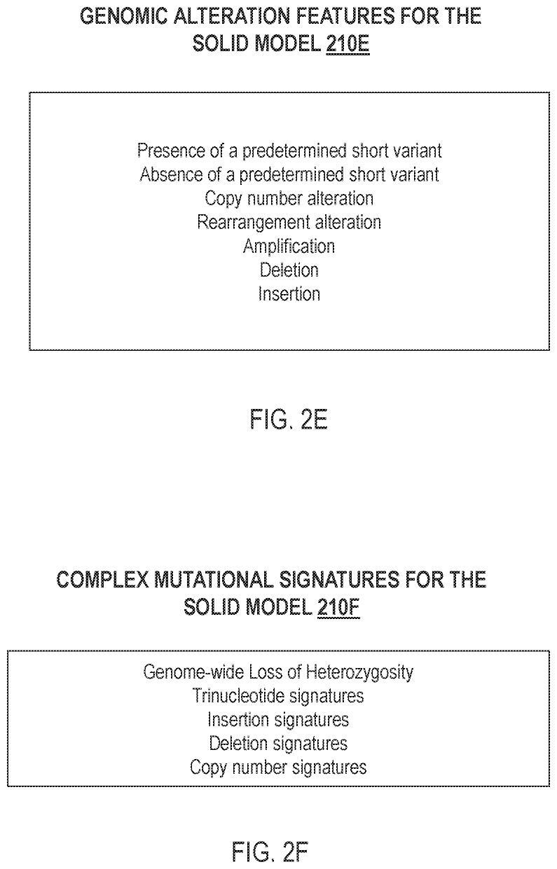

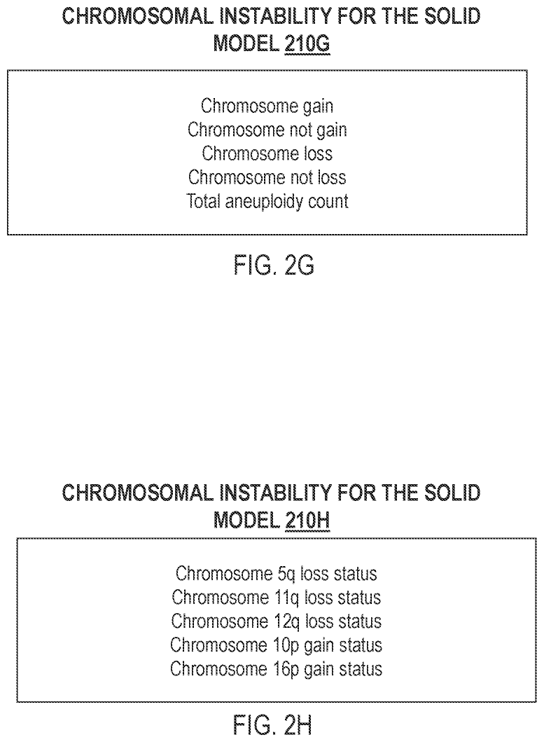

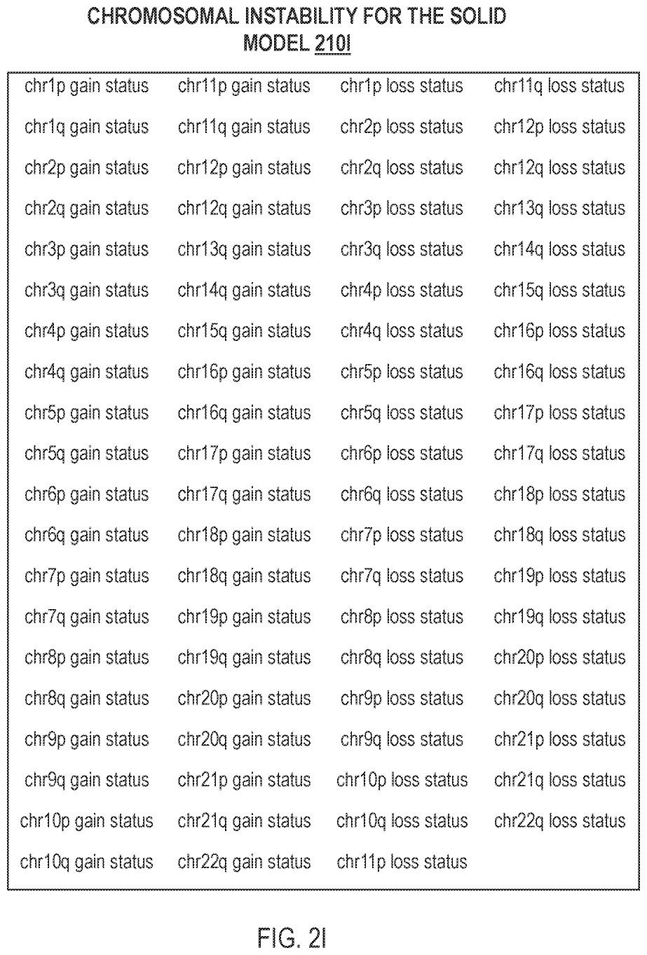

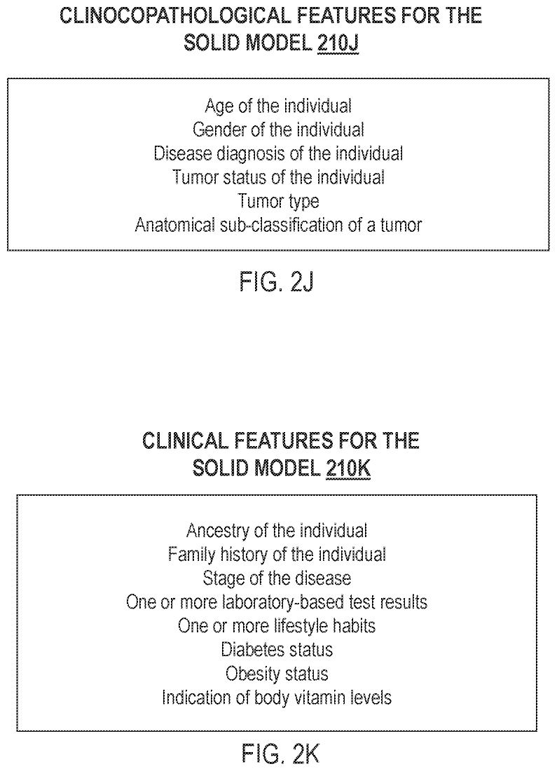

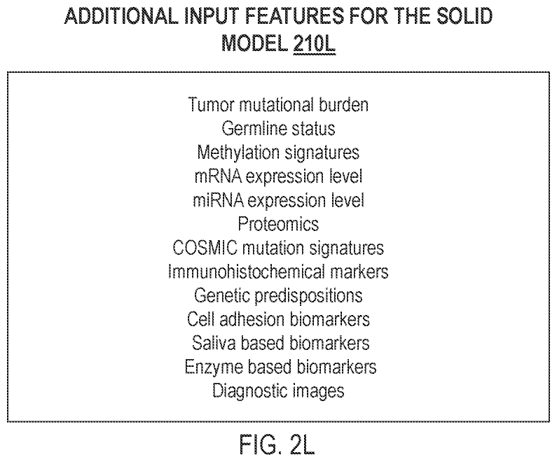

FIG. 2A illustrates exemplary input features 210A for a solid model in accordance with one or more embodiments of this disclosure. In one or more examples the set of input features 210A based on a solid sample can include one or more genomic alteration features, one or more complex mutational signatures, one or more chromosomal instability features, one or more clinicopathological features, one or more clinical features, and one or more additional features. A skilled artisan will understand that more or less input features can be included or omitted without departing from the scope of this disclosure.

FIGS. 2B-2E illustrates exemplary genomic alteration features 210B-210E for a solid model in accordance with one or more embodiments of this disclosure. In one or more examples, these genomic alteration features may correspond to alteration features expected to have a low prevalence and high correlation to the sequence read data.

In one or more examples, the genomic alterations can correspond to one or more known pathogenic alterations and/or one or more likely pathogenic alterations. The known pathogenic alterations and the likely pathogenic alterations may correspond to genomic alterations that are associated with a biologically activating alteration and/or an alteration that causes a change in a biological process that is known or likely to have an impact on a patient's disease status. As shown in FIG. 2B, in one or more examples, the genomic alteration features for the solid model may include, at least, TP53, ESR1, PIK3CA, ZNF703, and GATA3. As shown in FIG. 2C, in one or more examples, the genomic alteration features for the solid model can include, at least, CCNE1, EGFR, ERBB2, ERBB2 Amplification, ESR1, FGF3, FGFR1, GATA3, MAP2K4, MYC, PIK3CA, PTEN, RB1, TP53, and ZNF703.

As shown in FIG. 2D, in one or more examples, the genomic alteration features for the solid model can include, at least, AKT1, AKT2, AKT3, APC, AR, ARFRP1, ARIDIA, ATM, AURKA, BAP1, BCL2L1, BCL2L2, BRA, BRCA1, BRCA2, BRD4, CBFB, CCND3, CCNE1, CDH1, CDK12, CDK4, CDK6, CDKN2A, CDKN2B, CHEK2, CREBBP, CTNNA1, DNMT3A, EGFR, EMSY, EP300, ERBB2, ERBB2-Amplification, ERBB2 Short-Variant, ERBB3, ESR1, FANCA, FAS, FBXW7, FGF10 FGF12, FGF3, FGF6, FGFR1, FGFR2, FGFR4, GATA3, GNAS, HGF, IGF1R, IKBKE, IRS2, JAK2, KDM5A, KDM6A, KDR, KEL, KIT, KMT2D, KRAS, LYN, MAP2K4, MAP3K1, MCL1, MDM2, MDM4, MSH6, MUTYH, MYC, MYCL, NF1, NFKBIA, NKX2 1, NOTCH1, NOTCH2, NOTCH3, TRK1, PALB2, PBRM1, PDCD1LG2, PDGFRA, PIK3C2B, PIK3CA, PIK3CB, PIK3R1, PRKCI, PTEN, RAF1, RB1, RET, RICTOR, ROS1, RPTOR, SETD2, SF3B, SMAD4, SMARCA4, SOX2, SPEN, SRC, STK11, TBX3, TERC, TET2, TP53, TSC1, VEGFA, ZNF217, ZNF703. A skilled artisan will understand that these genomic alteration features are exemplary and that more genomic alteration features and/or different alteration features may be used without departing from the scope of this disclosure.

In one or more examples, determining whether a genomic alteration is present in the sequence read data may be helpful in determining a receptor status because particular genomic alterations may be associated with a known receptor status. For example, PIK3CA short variant alterations may be associated with ER positive tumors. As another example, TP53 short variant alterations may be associated with ER negative tumors. In yet another example, high level ERBB2 amplifications may be associated with HER2 positive tumors.

In one or more examples, the particular genomic alteration as well as the specific type of alteration may be included as an alteration feature. For example, as shown in FIG. 2E, the genomic alteration features can include, for each genomic alteration, the presence of a predetermined short variant, the absence of a predetermined short variant, a copy number alteration, a zygosity of the genomic alteration (e.g., homozygous or heterozygous), a somatic status of the genomic alteration, a germline status of the genomic alteration, a rearrangement alteration, an amplification, a deletion, or a combination thereof. In one or more examples, the genomic alterations 210E may be associated with a specific gene. This level of granularity in determining the type of alteration may be important for determining the biological processes that are relevant to disease. For example, HER2 short variants may be associated with ER positive subtypes while HER2 amplifications are not. Thus, an input feature corresponding to a presence of a HER2 variant without further specifying the type of alteration may not be as relevant to determining a receptor status. In this manner, distinguishing between the types of alterations improve the accuracy of the model for predicting the receptor status.

In one or more examples the genomic alteration features may be obtained via a computational pipeline for analyzing sequence read data. In one or more examples, the tumor fraction feature may be determined by the system based on information provided by the computational pipeline.

In one or more examples, the input features for the solid statistical model can include one or more complex mutational signature features. FIG. 2F illustrates exemplary complex mutational signature features 210F for a solid model, according to embodiments of the present disclosure. In one or more examples, the complex mutational signature features can include genome-wide loss of heterozygosity (gLOH), trinucleotide signatures, insertion signatures, deletion signatures, and copy number signatures. The genome-wide loss of heterozygosity may be associated with a loss of heterozygosity of the genome, typically caused by the loss of a gene associated with DNA repair. The trinucleotide signatures may be associated with one or more trinucleotide alterations in the sample. The insertion signatures may be associated with one or more insertion alterations in the sample. The deletion signatures may be associated with one or more deletion alterations in the sample. The copy number signatures may be associated with one or more copy number alterations in the sample.

In one or more examples, the input features for the solid statistical model can include one or more chromosomal instability features. FIGS. 2G-2I illustrate exemplary chromosomal instability features 210G-210I for a solid model. In one or more examples, these chromosomal instability features may correspond to features expected to have a low prevalence and high correlation to the sequence read data. FIG. 2G exemplary chromosomal instability features 210G, which may include chromosome gain, chromosome not gain, chromosome loss, chromosome not loss. In one or more examples, the chromosomal instability features can include a total aneuploidy count. As shown in FIG. 2H, in one or more examples, the chromosomal instability features can include at least, chromosome 5q loss status, chromosome 11q loss status, chromosome 12q loss status, chromosome 10p gain status, chromosome 16p gain status. In one or more examples, the chromosomal instability features can include at least, chromosome 1q loss status, chromosome 1p loss status, chromosome 3p loss status, chromosome 4p loss status, chromosome 5q loss status, chromosome 9p loss status, chromosome 10q loss status, chromosome 11q loss status, chromosome 12q loss status, chromosome 14q loss status, chromosome 15q loss status, chromosome 16p loss status, chromosome 16q loss status, chromosome 17q loss status, chromosome 19p loss status, chromosome 21q loss status, chromosome 22q loss status, chromosome 1q gain status, chromosome 2p gain status, chromosome 5p gain status, chromosome 5q gain status, chromosome 6q gain status, chromosome 7q gain status, chromosome 9p gain status, chromosome 10p gain status, chromosome 12q gain status, chromosome 16p gain status, chromosome 18p gain status, chromosome 20q gain status chromosome 21p gain status, and chromosome 21q gain status.

As shown in FIG. 2I, in one or more examples, the chromosomal instability features can include at least, chromosome 1p gain status, chromosome 1q gain status, chromosome2p gain status, chromosome 2q gain status, chromosome 3p gain status, chromosome 3q gain status, chromosome 4p gain status, chromosome 4q gain status, chromosome 5p gain status, chromosome 5q gain status, chromosome 6p gain status, chromosome 6q gain status, chromosome 7p gain status, chromosome 7q gain status, chromosome 8p gain status, chromosome 8q gain status, chromosome 9p gain status, chromosome 9q gain status, chromosome 10p gain status, chromosome 10q gain status, chromosome 11p gain status, chromosome 11q gain status, chromosome 12p gain status, chromosome 12q gain status, chromosome 13q gain status, chromosome 14q gain status, chromosome 15q gain status, chromosome 16p gain status, chromosome 16q gain status, chromosome 17p gain status, chromosome 17q gain status, chromosome 18p gain status, chromosome 18q gain status, chromosome 19p gain status, chromosome 19q gain status, chromosome 20p gain status, chromosome 20q gain status, chromosome 21p gain status, chromosome 21q gain status, chromosome 22q gain status, chromosome 1p loss status, chromosome 2p loss status, chromosome 2q loss status, chromosome 3p loss status, chromosome 3q loss status, chromosome 4p loss status, chromosome 4q loss status, chromosome 5p loss status, chromosome 5q loss status, chromosome 6p loss status, chromosome 6q loss status, chromosome 7p loss status, chromosome 7q loss status, chromosome 8p loss status, chromosome 8q loss status, chromosome 9p loss status, chromosome 9q loss status, chromosome 10p loss status, chromosome 10q loss status, chromosome 11p loss status, chromosome 11q loss status, chromosome 12p loss status, chromosome 12q loss status, chromosome 13q loss status, chromosome 14q loss status, chromosome 15q loss status, chromosome 16p loss status, chromosome 16q loss status, chromosome 17p loss status, chromosome 17q loss status, chromosome 18p loss status, chromosome 18q loss status, chromosome 19p loss status, chromosome 19q loss status, chromosome 20p loss status, chromosome 20q loss status, chromosome 21p loss status, chromosome 21q loss status, and chromosome 22q loss status.

In one or more examples, the input features for the solid statistical model can include one or more clinicopathological features. FIG. 2J illustrates exemplary clinicopathological features 210J for a solid model. In one or more examples, the clinicopathological features can include an age of the individual, a sex of the individual, a disease diagnosis of the individual, a tumor status of the individual, an anatomical sub-classification of a tumor, or a combination thereof. In one or more examples, these features can be obtained via a patient's medical records, and/or based on laboratory test results.

In one or more examples, the age of the individual can be based on an integer value of corresponding to the age in years of the individual. In one or more examples, the sex of the individual can be associated with the biological sex of the individual. In one or more examples, a disease diagnosis can be associated with a disease ontology for a particular disease, for example, a breast cancer diagnosis. In one or more examples, the disease diagnosis can be based on the International Classification of Diseases (ICD) codes (e.g., ICD-9 code or ICD-10 code).

In one or more examples, a tumor status of the individual may associated with the local or metastatic status of the sample. In one or more examples, an anatomical sub-classification of a tumor may be associated with breast cancer histology including, for example, but not limited to invasive ductal carcinoma, invasive lobular carcinoma, mixed ductal and lobular carcinoma, breast carcinoma, breast metaplastic carcinoma, breast myoepithelial carcinoma, breast carcinosarcoma, breast inflammatory carcinoma, breast mucinous carcinoma, breast papillary carcinoma, breast adenomyoepithelioma, breast phyllodes tumor etc.

Evaluating the clinicopathological features can provide further insight into the receptor status of an individual. For example, male breast cancer may be generally associated with a positive ER status. As another example, disease occurrence in younger patients may be associated with a negative ER status. As another example, invasive lobular breast cancer (ILC) may be associated with a positive ER status.

In one or more examples, the input features for the solid statistical model can include one or more clinical features. FIG. 2K illustrates exemplary clinical features 210K for a solid model. In one or more examples, clinical features may correspond to input features expected to have a low prevalence and high correlation to the sequence read data. As shown in the figure, in one or more examples, the clinical features 210K can include an ancestry of the individual, a family history of the individual, a stage of the disease, one or more laboratory-based test results, one or more lifestyle habits, a diabetes status, an obesity status, and an indication of body vitamin levels. In one or more examples, these features can be obtained via a patient's medical records, and/or based on laboratory test results.

Evaluating the clinical features can provide further insight into the receptor status of an individual. For example, different ancestry groups may be associated with a different preponderances of disease. For example breast cancer occurring in individuals with African ancestry is associated with a negative ER status. In one or more examples, a stage of the disease may be associated with a particular receptor status. For example, some diseases may present at higher stage at diagnosis (e.g., ILC may be associated with a positive ER status at diagnosis). As another example, lifestyle habits can play a role in developing certain cancers, e.g., a history of smoking or exercise. Obesity for example increases the risk of ER positive disease, e.g., breast cancer. As another example, the number of live births performed by an individual may influence the types of breast cancers that can develop.

In one or more examples, the input features for the solid statistical model can include one or more additional features not described above. FIG. 2L illustrates exemplary additional input features for a solid model 210L. In one or more examples, these additional input features may correspond to features expected to have a low prevalence and high correlation to the sequence read data. As shown in the figure, the exemplary additional input features for a solid model 210L can include, but not be limited to a tumor mutational burden (TMB) value, a germline status, methylation signatures, a mRNA expression level, a miRNA expression level, proteomics, COSMIC mutation signatures, immunohistochemical markers, genetic predispositions, cell adhesion biomarkers, saliva based biomarkers, enzyme based biomarkers, and diagnostic images. In some instances, the one or more input features may be associated with a germline status of one or more genes. Evaluating these additional features can provide further insight into the receptor status of an individual. For example, the germline status for certain genes may be associated with the incidence of disease. For example, germline BRCA1 alterations may be associated with developing triple negative breast cancer (TNBC), and further associated with a negative ER status, a negative HER2 status, and a negative PR status.

In one or more examples, the TMB value can correspond to a measure of genome-wide mutation frequency. For example, individuals with in invasive lobular carcinoma (ILC), may present with high TMB and may be ER positive. Accordingly, a high TMB value may be indicative of an ER positive status. ER negative tumors are maybe associated with intermediate TMB values.

In one or more examples, germline status may be indicative of whether an alteration is familial. In some examples, whether an alteration is germline or somatic may impact what type of disease the patient might develop. For example patients with germline BRCA1 tumors are frequently ER negative. Patients with germline CDH1 alterations are frequently ER positive.

In one or more examples, methylation signatures can correspond to patterns of methylation across the genome that can be indicative of chromatin state and gene expression. These patterns may differ across tumor types and can be informative as to the underlying cell state of tumor and the receptor status (e.g., ER positive versus ER negative).

In one or more examples, RNA signatures can be used to identify characteristics of a tumor. For example, in breast cancer multiple RNA signatures (e.g., PAM50) and custom signatures may be used. In one or more examples, the system can cluster similar states, and identify if a tumor is ‘basal like,’ for example, which can be indicative of an ER negative status. In one or more examples, the level of ESR1 expression or ERBB2 (HER2) expression can be informative for the receptor status.

In one or more examples, the miRNA expression level can be associated with regulatory RNAs. The presence or absence of miRNA could indicate a state of the tumor cells (e.g., positive or negative receptor status).

In one or more examples, proteomics measures the levels of different proteins in the cell. The specific expression levels of certain proteins can help indicate cell state. For example, tumor cells with a lot of ER protein may be indicative of an ER positive status.

In one or more examples, COSMIC mutation signatures correspond to signatures of underlying mutational processes that can be measured by looking at the context of mutations. There are a number of COSMIC signatures including those derived from point mutations, indels, and dinucleotide substitutions. In breast cancer, for example, apolipoprotein B mRNA editing catalytic polypeptide (APOBEC) signatures are more common and are enriched in ER positive disease. In some examples, homologous repair deficiency (HRD) signatures may be indicative of ER negative disease.

In one or more examples, immunohistochemical markers can measure protein expression and localization in tissue slides. The presence or absence of other markers (e.g., E-cadherin) can be indicative of a diagnostic receptor gene status. For example, tumors that have lost membrane-localized E-cadherin may be more likely to have an ER positive status.

In one or more examples, genetic predispositions can include disorders or family histories that predispose an individual to cancer. In some instances, the type of cancer developed by individuals may have a bias in the receptor status. For example, an individual may be more likely to have an ER negative status if there is a family history of ER-negative breast cancer.

In one or more examples, cell adhesion biomarkers can correspond to cell surface molecules like E-cadherin or cytokeratins (e.g., CK8/18). For example, cytokeratins can indicate an epithelial and/or mesenchymal state which can correlate with ER status.

In one or more examples, saliva based biomarkers and enzyme based biomarkers can indicate possible predispositions to certain disease types. For example, individuals with biomarkers indicative of obesity or diabetes may be predisposed to certain types of breast cancers, which can be indicative of receptor status.

In one or more examples, the exemplary additional features may be obtained via a computational pipeline for analyzing sequence read data. For example, sequence read data from the computational pipeline may be used, but is not limited to determining the tumor mutational burden, germline status, COSMIC mutation signatures, etc. In one or more examples, the exemplary additional features may be obtained via tests administered by a clinician, e.g., diagnostic images, saliva based biomarkers, urinalysis, etc. For example, urinalysis can detect features such as red blood cell (RBC) count, sugar content, circulating tumor cells, etc.

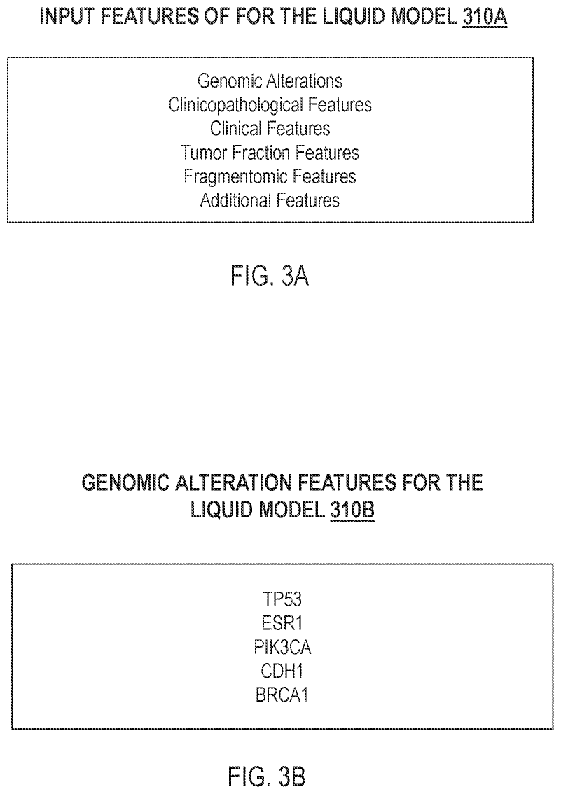

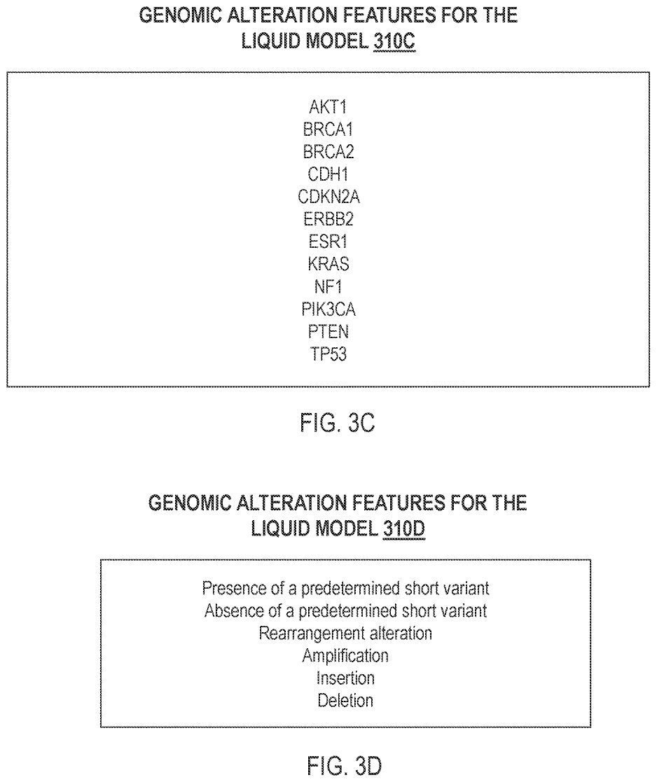

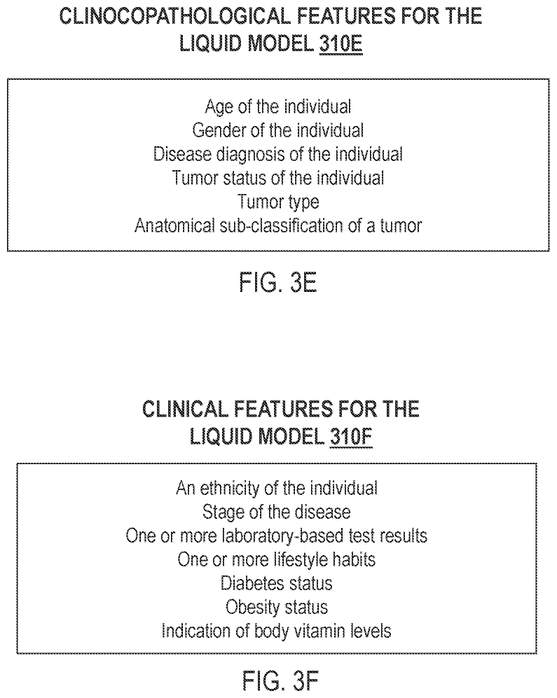

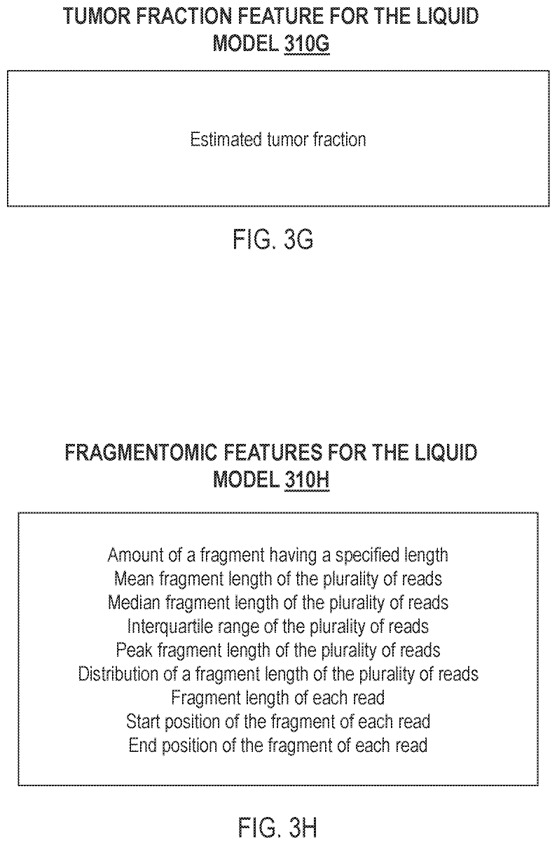

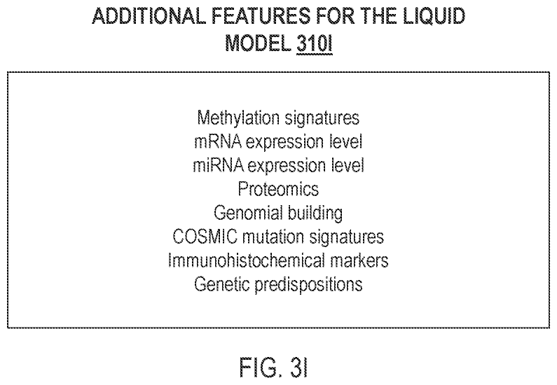

FIG. 3A illustrates exemplary input features 310A for a liquid model in accordance with one or more embodiments of this disclosure. In one or more examples the set of input features 310A for a liquid sample can include one or more genomic alteration features, one or more clinicopathological features, one or more clinical features, one or more tumor fraction features, one or more fragmentomic features, and one or more additional features. A skilled artisan will understand that more or less input features can be included without departing from the scope of this disclosure. In one or more examples, the exemplary input features 310A for a liquid model may differ from the exemplary input features 210A for a solid model. Some of these differences may be because of the differences in tumor purity in liquid biopsy samples and solid biopsy samples. Liquid samples typically have less shed so calling amplifications and deletions may be more difficult than with solid samples. Additionally, for liquid samples comprising blood, the sample may integrate all sites that shed and different patterns of resistance alterations may be observed (e.g., the system may observe polyclonal alterations or alterations from multiple resistance pathways). Additionally, solid and liquid samples may be associated with different patterns of baiting (e.g., with respect to genes baited in an assay and/or the level of coverage).

FIGS. 3B-3E illustrates exemplary genomic alteration features 210B-210E for a liquid model in accordance with one or more embodiments of this disclosure. In one or more examples, these genomic alteration features may correspond to alteration features expected to have a low prevalence and high correlation to the sequence read data.

In one or more examples, the genomic alterations can correspond to one or more known pathogenic alterations and/or one or more likely pathogenic alterations. The known pathogenic alterations and the likely pathogenic alterations may correspond to genomic alterations that are associated with a biologically activating alteration that is known or likely to have an impact on a patient's disease status. As shown in FIG. 3B, in one or more examples, the genomic alteration features for the liquid model may include, at least, TP53, ESR1, PIK3CA, CDH1, and BRCA1. As shown in FIG. 3C, in one or more examples, the genomic alteration features for the liquid model can include, at least, AKT1, BRCA1, BRCA2, CDH1, CDKN2A, ERBB2, ESR1, KRAS, NF1, PIK3CA, PTEN, and TP53.

In one or more examples, the genomic alteration as well as the specific type of alteration may be included as an alteration feature. For example, as shown in FIG. 3D, the genomic alteration features 310D for a liquid model can include, for each genomic alteration, the presence of a predetermined short variant, the absence of a predetermined short variant, a copy number alteration, a zygosity of the genomic alteration (e.g., homozygous or heterozygous), a somatic status of the genomic alteration, a germline status of the genomic alteration, a rearrangement alteration, an amplification, a deletion, or a combination thereof. In one or more examples, the genomic alterations 310D may be associated with a specific gene. As discussed above, this level of granularity in determining the type of alteration may be important for determining the biological processes that are relevant to disease.

In one or more examples the genomic alteration features may be obtained via a computational pipeline for analyzing sequence read data. In one or more examples, the tumor fraction feature may be determined by the system based on information provided by the computational pipeline.

In one or more examples, the input features for the liquid model can include one or more clinicopathological features. FIG. 3E illustrates exemplary clinicopathological features 310E for a liquid model. In one or more examples, the clinicopathological features can include an age of the individual, a sex of the individual, a disease diagnosis of the individual, a tumor status of the individual, a tumor type, an anatomical sub-classification of a tumor, or a combination thereof. As discussed above with respect to the solid model, in one or more examples, these features can be obtained via a patient's medical records, and/or based on laboratory test results.

In one or more examples, the input features for the liquid model can include one or more clinical features. FIG. 3F illustrates exemplary clinical features 310F for a liquid model. In one or more examples, clinical features may correspond to input features expected to have a low prevalence and high correlation to the sequence read data. As shown in the figure, in one or more examples, the clinical features 310F can include an ancestry of the individual, a family history of the individual, a stage of the disease, one or more laboratory-based test results, one or more lifestyle habits, a diabetes status, an obesity status, and an indication of body vitamin levels. As discussed above with respect to the solid model, in one or more examples, these features can be obtained via a patient's medical records, and/or based on laboratory test results.

In one or more examples, the input features for the liquid model can include one or more tumor fraction features. FIG. 3G illustrates an exemplary tumor fraction feature 310G for a liquid model. As shown in the figure, the exemplary tumor fraction feature can include an estimated tumor fraction of the sample, an estimated degree of polyclonality, or a combination thereof. The estimated tumor fraction may be based on a level of shed of a tumor, which can be indicative of a receptor status. In one or more examples, the estimated tumor fraction can be expressed as a percentage of circulating cell-free DNA. In one or more examples, the tumor fraction feature may be obtained via a computational pipeline for analyzing sequence read data. In one or more examples, the tumor fraction feature may be determined, directly or indirectly using multiple computational processes (e.g., using fragmentomic length, variant allele fraction, and flow cytometry), by the system based on information provided by the computational pipeline.

In one or more examples, the input features for the liquid model can include one or more fragmentomic features. FIG. 3H illustrates exemplary fragmentomic features 310H for a liquid model. As shown in the figure, the exemplary fragmentomic features 310H can include one or more of an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a start position of a fragment for the selected plurality of reads, and an end position of a fragment for the selected plurality of reads. The provided examples of fragmentomic characteristics 310A is not exhaustive and a skilled artisan would understand that additional fragmentomic characteristics could be determined for the plurality of reads without departing from the scope of this disclosure.

In some instances, the amount of a fragment having a specified length can correspond to a total amount (i.e., count) of fragments at the specified length. For example, for a particular sample, the total number of fragments that have a length below 100 bp, a length of 100 bp, a length of 101 bp, a length of 102 bp, . . . a length of 550 bp, and a length greater than 550 bp. A skilled artisan will understand that these examples of specified lengths are exemplary and is not intended to limit the scope of the disclosure. For example, the specified length can correspond to a specific number of base pairs, a range of number of base pairs, or a combination thereof.

In some instances, the amount of a fragment having a specified length can correspond to a relative amount of fragments of a selected plurality of reads (e.g., reads overlapping with the alteration or gene of interest) corresponding to a specified length. In some examples, the amount of a fragment having a specified length can comprise a fraction. For example, the amount of a fragment having a specified length can be determined based on the number of fragments with a specified length (e.g., a length below 50 bp, a length of 50 pb, a length of 51 bp, . . . , a length of 550 bp, and a length greater than 550 bp) divided by the number of the selected plurality of reads to determine the amount of the fragments having a specified length. In some examples, the amount can correspond to a selected plurality of reads that include an alteration and/or a selected plurality of reads that include a wild type gene. A skilled artisan will understand that these examples of specified lengths are exemplary and is not intended to limit the scope of the disclosure.

In some instances, the mean fragment length of the selected plurality of reads can correspond to an average fragment length of the selected plurality of reads. In some instances, the median fragment length of the selected plurality of reads can correspond to the middle fragment length value of a sorted list of the fragment lengths of the selected plurality of reads. In some instances, the interquartile range of fragment lengths of the plurality of reads can correspond to a first fragment length value associated with the 25th percentile of the fragment lengths of the selected plurality of reads and a second fragment length value associated with the 75th percentile of the fragment lengths of the selected plurality of reads. In some instances, the peak fragment length can correspond to the mode or the fragment length value that appears most frequently in the length characteristics for the selected plurality of reads. In some instances, the system can determine more than one peak fragment length. In some instances, the distribution of the fragment length can correspond to a summary statistics characterizing the distribution, e.g., maximum value, minimum value, standard deviation, shape, etc.

In one or more examples, the fragmentomic features may be obtained via a computational pipeline for analyzing sequence read data. In one or more examples, the fragmentomic features may be determined by the system based on information provided by the computational pipeline.

In one or more examples, the input features for the solid statistical model can include one or more additional features not described above. FIG. 3I illustrates exemplary additional input features 310I for a liquid model. In one or more examples, these additional input features may correspond to features expected to have a low prevalence and high correlation to the sequence read data. As shown in the figure, the exemplary additional input features 310I for a liquid model can include, but not be limited to a tumor mutational burden, germline status, methylation signatures, a mRNA expression level, a miRNA expression level, proteomics, COSMIC mutation signatures, immunohistochemical markers, genetic predispositions, cell adhesion biomarkers, saliva based biomarkers, enzyme based biomarkers, a diagnostic image, and a germline status for certain genes.

As discussed above, the exemplary additional features may be obtained via a computational pipeline for analyzing sequence read data, e.g., to estimate the tumor mutational burden, germline status, COSMIC mutation signatures, etc. In one or more examples, the exemplary additional features may be obtained via tests administered by a clinician, e.g., diagnostic images, saliva based biomarkers, etc.

The provided examples of input features for the solid and liquid models discussed above and provided in FIGS. 2A-2L and FIGS. 3A-3I are not exhaustive and a skilled artisan would understand that additional input features could be used without departing from the scope of this disclosure.

At step 106 in FIG. 1, the system can determine values for one or more input features corresponding to the set of input features based on the sequence read data. For example, the values (e.g., input feature values) for the one or more input features can be determined based on the presence of each of one or more of the input features of the set of input features in the sequence read data.

For example, for one or more of the genomic alteration features 210B-210E for the solid model, the system may determine corresponding input feature values. In one or more examples, the genomic alteration features, e.g., 210B-210E, may be associated with binary values indicative of a presence of a particular genomic alteration (e.g., TP54, ESR1, etc.) and/or indicative of a particular type of alteration (e.g., short variant, copy-number alteration, rearrangement, etc.). In one or more examples, the input feature values corresponding to one or more genomic alteration features 310B-310D for the liquid model may also be associated with binary values indicative of a presence of a particular genomic alteration (e.g., TP53, ESR1, etc.) and/or indicative of a particular type of alteration (e.g., short variant, copy-number alteration, rearrangement, etc.).

In one or more examples, for the one or more complex mutational signature features 210F discussed above, the system may determine corresponding input feature values. In one or more examples, the complex mutational signature features may be determined using the methods of Macintyre et al. incorporated herein by reference. (See, e.g., G Macintyre et al.: Copy number signatures and mutational processes in ovarian carcinoma. Nat Genet 2018, 50(9):1262-1270, hereby incorporated in its entirety).

In one or more examples, a gLOH quantification may be associated with a binary value indicative of the presence of high gLOH or a float point number indicative of a measure of the gLOH. The gLOH quantification can correspond to a measure of focal genome-wide loss of heterozygosity, a biomarker for HRD and genomic instability. In some instances, a high gLOH value may be indicative of an ER negative status. In one or more examples, trinucleotide signatures, indel signatures, and copy number signatures may be associated with a binary value indicative of the presence or absence of the respective signature. In one or more examples, copy number features can be extracted using the methods of Macintyre et al. In one or more examples, the trinucleotide signatures and indel signatures may be based on the COSMIC signatures described above.

In one or more examples, for one or more of the chromosomal instability features 210G-210I discussed above, the system may determine corresponding input feature values. In one or more examples, the genomic alteration features, e.g., 210B-210E, may be associated with binary values indicative of a presence of a particular chromosomal instability feature (e.g., chromosome gain, chromosome not gain, chromosome loss, chromosome not loss, total aneuploidy count, etc.) and/or indicative of a gain or loss status of a particular chromosome (e.g., chromosome 5q loss status, chromosome 11q loss status, chromosome 12q loss status, etc.).

In one or more examples, for one or more of the clinicopathological features 210J and 310E discussed above, the system may determine corresponding input feature values. In one or more examples, age may be associated with any positive value. In one or more examples, sex may be associated with a binary value. In one or more examples, a disease diagnosis, a tumor status, a tumor type, and an anatomical sub-classification of the tumor may be associated with a categorical string.

In one or more examples, for one or more of the clinical features 210K and 310F discussed above, the system may determine corresponding input feature values. In one or more examples, ancestry may be associated with a categorical string (e.g., EAS, SAS, AFR, AMR, EUR, etc.) or float value for admixture in each ancestry group. The number and types of ancestry groups is not intended to limit the scope of the disclosure. In one or more examples, the stage of the disease can be associated with a categorical string (e.g., IA, II, etc.). In one or more examples, lifestyle habits may be associated with a series of binary values (e.g., overweight: yes/no, smoking: yes/no, live birth: (yes/no)) or one or more floating point values or integers (e.g., number of live births: integer, number of pack years smoked: integer or floating point value, etc.) indicative of the lifestyle habits of the individual. In one or more examples, an obesity status may be associated with a binary value (e.g., yes, no) or a floating point value (e.g., BMI). In one or more examples, a body vitamin level may be associated with a floating point value or a categorical string (e.g., high) indicative of the amount of body vitamin level in the individual. In one or more examples, family history may be associated with a categorical string or a binary value.

In one or more examples, the tumor fraction feature 310G may be associated with a percentage or a floating point value indicative of an estimated tumor fraction in the sample. In one or more examples, the fragmentomic features may be associated with an integer and or floating point value or fraction, as discussed above.

In one or more examples, for one or more of the additional features (e.g., 210L and 310I) discussed above, the system may determine corresponding input feature values. To the extent that the following features are described as associated with a particular type of value (e.g., binary, non-binary, integer, floating point, categorical, etc.) a skilled artisan will understand that these examples are not intended to limit the scope of this application and other types of values may be used without departing from the scope of this disclosure.

In one or more examples, methylation signatures may be associated with a binary value indicative of a presence of a methylation signature, for example the methylation status of the BRCA1 promoter. In one or more examples, the methylation signatures may be associated with a non-binary value and/or any positive value. In one or more examples, an mRNA expression level may be associated with an integer or floating point value indicative of the mRNA expression level, for example a PAM50 classification. In one or more examples, a miRNA expression level may be associated with an integer or floating point value indicative of the miRNA expression level. In one or more examples, proteomics may be associated with a high or low expression of a protein indicative of pathway activity. In one or more examples, COSMIC mutation signatures may be associated with a probability value indicative of the presence of a respective mutation signature, for example the presence of an APOBEC trinucleotide signature or a HRD indel signature. In one or more examples, immunohistochemical markers may be associated with a binary value, a percentage, or a category (e.g., negative, low, high) indicative of the presence of a respective immunohistochemical marker (e.g., E-cadherin membrane staining). In one or more examples, genetic predispositions may be associated with a binary value indicative of one or more genetic predispositions (e.g., a family history of triple negative breast cancer or carrier status for germline CDH1 that can predispose to ER+ disease). In one or more examples, cell adhesion biomarkers may be associated with a binary value, a floating point value, and the like, indicative of the presence of a respective cell adhesion biomarker (e.g., cytokeratin or cadherin statuses). In one or more examples, saliva based biomarkers may be associated with a binary value indicative of the presence of a respective saliva based biomarker. In one or more examples, enzyme based biomarkers may be associated with a binary value indicative of the presence of a respective enzyme based biomarkers. In one or more examples, the input value may be associated with an image file (e.g., JPEG, TIFF, raw image format, BMP, etc.) of the diagnostic image.

At step 108 in FIG. 1, the system can input the one or more input feature values into the statistical model. In one or more examples, the statistical model can be a trained machine learning model. For example, the system can input one or more of the one or more input feature values into a trained machine learning model. In one or more examples, the trained machine learning model may be a random forest model.

In one or more examples, the statistical model may be part of a machine learning process. In one or more examples, the machine learning model can include an artificial intelligence (“AI”) learning model. In some instances, the machine learning model can be at least one of a supervised model or an unsupervised model. In one or more examples, the machine learning model can include one or more machine learning models, such as regression-based models (e.g., including but not limited to logistic regression, nearest neighbor regression, proportional hazards regression etc.), regularization-based models (e.g., including but not limited to elastic net, ridge regression etc.), instance-based models (e.g., including but not limited to support vector machines, k-nearest neighbor etc.), Bayesian-based models (e.g., including but not limited to naïve-based, Gaussian naïve-based etc.), clustering-based models (e.g., including but not limited to expectation maximization), ensemble-based models (e.g., including but not limited to adaboost, bagging, gradient boosting machines etc.), and neural network-based models (e.g., including but not limited to backpropagation, stochastic gradient descent etc.).

In one or more examples, the model can be trained to predict the receptor status of an individual based on an output of the statistical model. In one or more examples, the output of the statistical model may be indicative of a receptor status. In one or more examples, the output of the statistical model may include a score indicative of a probability of a positive receptor status. In one or more examples, the output of the statistical model may include a score indicative of a probability of a negative receptor status.

At step 110 in FIG. 1, the system can predict the receptor status of the individual based on an output of the trained statistical model. In one or more examples, the system can predict the receptor status of the individual by comparing a score (e.g., probability score) output by the statistical model to one or more predefined thresholds. For example, the system can compare the score to one or more predefined thresholds and determine whether the sample has a positive receptor status or a negative receptor status. In one or more examples, the predefined threshold may be 0.5. In one or more examples, the predefined threshold may be in a range of 0.05 to 0.95. In one or more examples, the thresholds may be configured to maximize different measures of prediction accuracy in a binary classification problem (e.g., the F1 score, F2 score, Matthew's correlation coefficient, Youden index, Cohen's kappa). In one or more examples, the thresholds can correspond to a value between zero and one.

In one or more examples, a first threshold of the one or more thresholds can be determined such that if the score is above the threshold, then the sequence read data is predicted to have a positive receptor status. In such examples, if the score is below the threshold, then the system can predict that the sequence read data of the sample has a non-positive receptor status.

In one or more examples, the one or more predetermined thresholds can be determined by maximizing or minimizing a function of sensitivity and specificity (such as the sum) For example, a loss function associated with performance metrics (e.g., whether the score corresponds to an accurate prediction) can be maximized or minimized. In some examples, the threshold can be set to maximize sensitivity and specificity.

In one or more examples, the one or more predetermined thresholds can be determined based on the area under the prediction function's receiver operating characteristic (ROC) curve. The area under a receiver operating characteristic curve can be used in statistics to measure the prediction accuracy of a binary classifier system. In one or more examples, the thresholds can be determined using one or more statistical techniques combined with predetermined confidence levels.

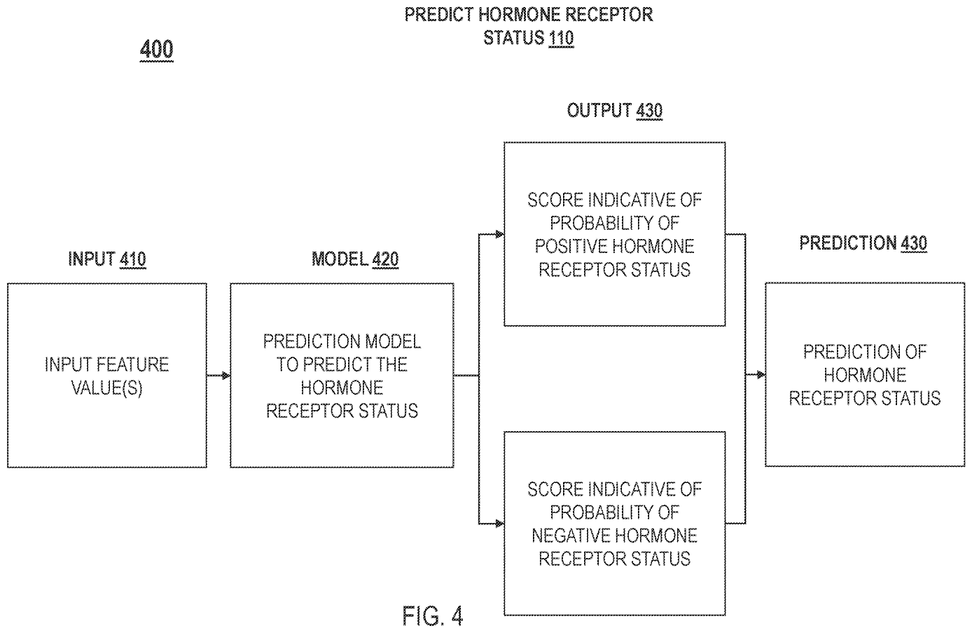

FIG. 4 is a diagram illustrating a process of predicting a receptor status using a statistical model, according to embodiments of the present disclosure. As shown in the figure, input data 410 corresponding to one or more input feature values (e.g., associated with solid model genomic alteration features 210B-210E, complex mutational signature features 210F, chromosomal instability features 210G-210I, clinicopathological features 210J, clinical features 210K, and additional features 210L; or liquid model genomic alteration features 310B-310D, clinicopathological features 310E, clinical features 310F, tumor fraction features 310G, fragmentomic features 310H, and additional features 310I) can be input into model 420. In one or more examples, the input data 410 can be associated with the input feature values described above with respect to step 106.

The model 420 can be a statistical model, such as a trained machine learning model configured to predict a receptor status of a sample. The model 420 can then output 430 one or more scores indicative of a receptor status. As shown in the figure, the output 430 of the model can include one or more scores associated with, for example, an indication of a positive receptor status and an indication of a negative receptor status. In one or more examples, the model 420 may output a single score (e.g., a score indicative of a positive receptor status or a score indicative of a negative receptor status). In one or more examples, model 420 can be associated with process 100.

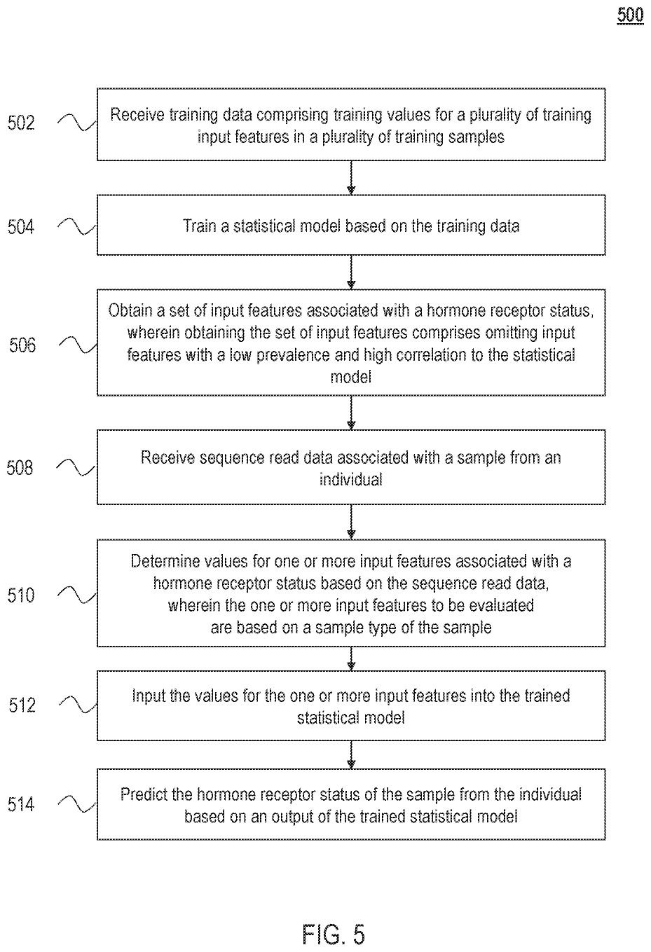

FIG. 5 provides a non-limiting example of a process 500 for predicting a receptor status of a sample from an individual. The process 500 includes one or more steps related to training a statistical model to predict the receptor status.

Process 500 can be performed, for example, using one or more electronic devices implementing a software platform. In some examples, process 500 is performed using a client-server system, and the blocks of process 500 are divided up in any manner between the server and a client device. In other examples, the blocks of process 500 are divided up between the server and multiple client devices. Thus, while portions of process 500 are described herein as being performed by particular devices of a client-server system, it will be appreciated that process 500 is not so limited. In other examples, process 500 is performed using only a client device or only multiple client devices. In process 500, some blocks are, optionally, combined, the order of some blocks is, optionally, changed, and some blocks are, optionally, omitted. In some examples, additional steps may be performed in combination with the process 500. Accordingly, the operations as illustrated (and described in greater detail below) are exemplary by nature and, as such, should not be viewed as limiting.

At step 502 in FIG. 5, the system can receive training data including a plurality of training input feature values corresponding to a plurality of training samples. In one or more examples, the training data can further include a receptor status of the respective training sample. In one or more examples, the plurality of input training feature values may be determined based on a plurality of training samples. In one or more examples, the types of training input features may be based on a sample type, e.g., solid sample or liquid sample. In one or more examples, the input training feature values may be associated with one or more genomic alteration features, complex mutational signature features, chromosomal instability features, clinicopathological features, clinical features, or additional features. In one or more examples, the input training feature values may be associated with one or more genomic alteration features, clinicopathological features, clinical features, tumor fraction features, fragmentomic features, or additional features.

In one or more examples, the plurality of input training feature values may be determined based on via a computational pipeline for analyzing sequence read data, e.g., to determine the genomic alteration feature values, complex mutation signature features, chromosomal instability features, and/or additional features. In one or more examples, one or more of the clinicopathological features, clinical features, and additional features may be obtained via a patient's medical records. In one or more examples, one or more additional features may be determined via tests administered by a clinician, e.g., diagnostic images, saliva based biomarkers, etc.

At step 504 in FIG. 5, the system can train a statistical model based on the training data. The model can be trained to predict a score indicative of a receptor status. For example, the model can be trained to determine one or more scores associated with an indication of a positive receptor status and an indication of a negative receptor status. In one or more examples, the model, e.g., model 420, may output a single score (e.g., a score indicative of a positive receptor status or a score indicative of a negative receptor status).

In one or more examples, the model may also be configured to output an indication of a relevance of the one or more training input features. For example, for each of the training input features, the model can output a respective score that indicates the relative importance of the training input feature in determining the score indicative of the receptor status. In one or more examples, the indication of the relevance of the one or more training input features may be used to adjust the weights of the model associated with the training input features.

In one or more examples, models may be separately trained based on the receptor status and the sample type. For example, a first model may be trained to determine a PR status for liquid biopsy samples and a second model may be trained to determine a PR status for solid biopsy samples. As another example, a third model may be trained to determine an ER status for liquid biopsy samples and a fourth model may be trained to determine an ER status for solid biopsy samples. As another example, a fifth model may be trained to determine a HER2 status for liquid biopsy samples and a sixth model may be trained to determine a HER2 status for solid biopsy samples. As another example, a seventh model may be trained to determine an androgen receptor status for liquid biopsy samples and an eighth model may be trained to determine an androgen receptor status for solid biopsy samples. In one or more examples, each of the models may be trained separately. In some instances the models may be trained simultaneously via, for example, a multi-task learning structure. A skilled artisan will understand that the models enumerated above are exemplary and additional or less models may be trained to determine a receptor status of various hormones according to embodiments of the present disclosure.

In one or more examples, embodiments of the present disclosure can further include fine tuning a machine learning tumor type classification by employing a statistical model (e.g., a deep learning model, including but not limited to convolutional neural networks, recurrent neural networks, auto-encoders etc.). In one or more examples, such models may be trained on breast tumor diagnostic images such as histopathological images, radiological images, magnetic resonance imaging, ultrasound imaging, X-ray imaging (mammogram), bone scans, CT scans, PET scans, etc.

In one or more examples, modifying or fine-tuning the classification model may include adding human interpretable features (HIFs) and phenotypes relevant to the receptor status of the tumor. The HIFs may be extracted from imaging data and used as additional features in the model for predicting the receptor status of a patient. The HIFs can be extracted from histopathological images using machine learning methods, such as, deep learning machine learning methods.

For example, the system may input a diagnostic image associated with an individual into a second statistical model (e.g., trained on diagnostic images) and determine a score indicative of a tumor classification based on the diagnostic image. In one or more examples, the tumor classification can be used as an input to the model for predicting a receptor status (e.g., models 420 and 620).

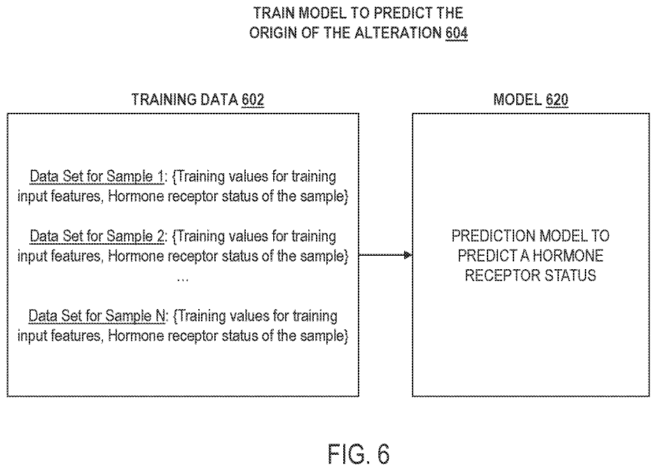

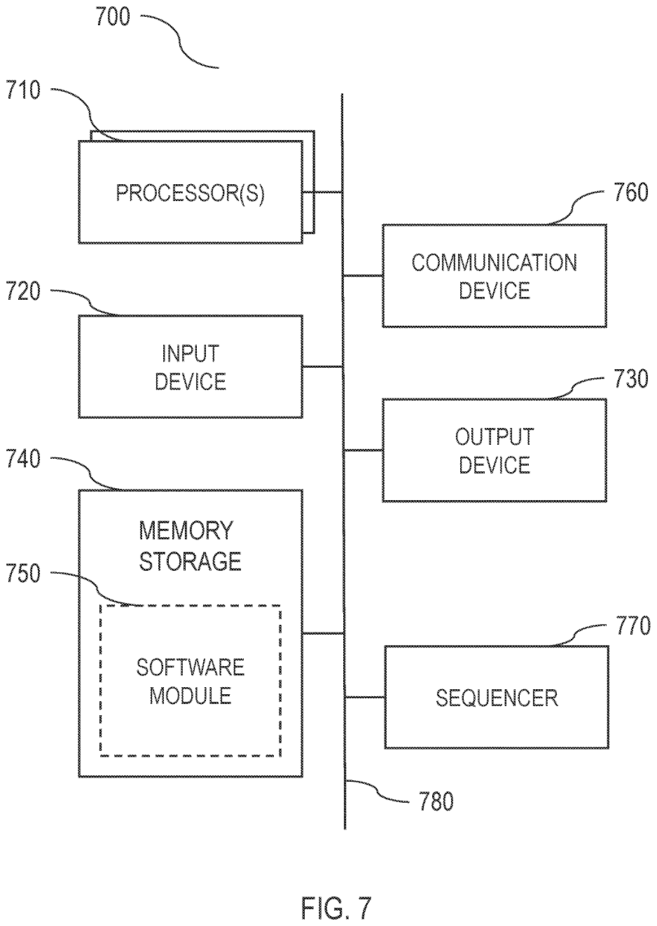

FIG. 6 illustrates a non-limiting example of a diagram for a process 600 for training a model 620 to predict a receptor status, according to embodiments of this disclosure. In one or more examples, process 600 can correspond to Step 504 of process 500. In one or more examples, the training at step 504 can be applied to train model 420 described with respect to FIG. 4. As shown in FIG. 6, training data 602 can be input into model 620.

The training data 602 can include one or more data sets corresponding to a plurality of samples (e.g., samples from individuals or patients). Each data set can include training values associated with a plurality of training input features and a corresponding label indicative of a receptor status of the sample. In one or more examples, the training data may be associated with a sample type, e.g., solid biopsy sample type or liquid biopsy sample type. In one or more examples, the training data 602 may include the receptor status of a particular receptor, e.g., ER, PR, HER2, androgen receptor. In such examples, the model 620 may be configured to predict the receptor status for a particular receptor for a particular sample type. In one or more examples, a statistical model can be built using a training data associated with a first sample type (e.g., solid training samples) and the model can be validated using training data associated with a second sample type (e.g., liquid training samples). In one or more examples, the statistical model can be trained and validated using the same sample type (e.g., solid training samples).

In one or more examples, the score of the model, e.g., model 420, 620, can be determined based on a weighted evaluation of the training data 602 (e.g., training values for genomic features associated with a receptor status). For example, the training can assign weights to the different training input feature values.

At step 506 in FIG. 5, the system can obtain a set of input features associated with a receptor status, wherein obtaining the set of input features comprises omitting input features with a low prevalence and high correlation to the statistical model. For example, based on the indication of the relevance of the one or more training input features, the system can determine which input features are associated with a low prevalence, but nonetheless have a high correlation to the score indicative of the receptor status. Such input features may be excluded from the statistical model in order to improve the accuracy of the prediction as discussed above.

In one or more examples, the input features expected to have a low prevalence and/or high correlation to the statistical model may be omitted from the set of input features to be used in the trained model. By omitting low prevalence and/or highly correlated features, the system may improve the reliability of the model. For example, an input feature that is found in less than one percent of tumors may not provide valuable information to predict a receptor status and may potentially skew the data. In one or more examples, omitting the low prevalence and highly correlated training input features may reduce the number of input features by about 40%. In one or more examples, omitting the low prevalence and highly correlated training input features may reduce the number of input features by about 25%, 30%, 35%, 45%, 50%, 55%, 60%, 65% 70%, 75%, 80%, 85%, 90%, or 95%.

In one or more examples, training input features with a prevalence less than a predetermined prevalence threshold (e.g., 1%) may be determined to have a low prevalence. In one or more examples, the predetermined prevalence threshold may be in a range of 0.1%-5.0%. In one or more examples, training input features with a correlation greater than a predetermined correlation threshold (e.g., 90%) may be determined to be highly correlated. In one or more examples, the predetermined correlation threshold may be in a range of 50%-95%.

At step 508 in FIG. 5, the system can receive sequence read data associated with a sample from an individual. In one or more examples, step 508 can correspond to step 102 of process 100 described above. At step 510 in FIG. 5, the system can determine one or more input feature values corresponding to the set of input features associated based on the selected plurality of reads and based on a sample type associated with of the sample. In one or more examples, step 510 can correspond to step 106 of process 100 described above. At step 512 in FIG. 5, the system can input the one or more input feature values into the trained statistical model. In one or more examples, step 512 can correspond to step 108 of process 100 described above. At step 514 in FIG. 5, the system can predict the receptor status of the individual based on an output of the trained statistical model. In one or more examples, step 514 can correspond to step 110 of process 100 described above.

In one or more examples, the sequence read data (e.g., obtained in step 102 of process 100 and/or step 506 of process 500 ) may be obtained from a gene panel. In some instances, the gene panel may comprise at least 1, at least 2, at least 3, at least 4, at least 5, at least 6, at least 7, at least 8, at least 9, at least 10, at least 20, at least 30, at least 40, or more than 40 genes.

In some instances, the disclosed methods may be used to determine a receptor status of an individual by assessing one or more input features associated with at least 1, at least 2, at least 3, at least 4, at least 5, at least 6, at least 7, at least 8, at least 9, at least 10, at least 20, at least 30, at least 40, or more than 40 gene loci.

In some instances, the disclosed methods may be used to identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining a receptor status of an individual may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for determining a receptor status of an individual may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for determining a receptor status of an individual may be used to select a subject (e.g., a patient) for a clinical trial based on the score indicative of a receptor status based on alterations present at one or more gene loci. In some instances, patient selection for clinical trials based on, e.g., identification of a receptor status, may accelerate the development of targeted therapies and improve the healthcare outcomes for treatment decisions.

In some instances, the disclosed methods for determining a receptor status of an individual may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise therapies that target the relevant receptor (e.g., AR, PR, ER, HER2 receptor) and/or therapies that target hormone production, (e.g., hormone therapies). In one or more examples, the therapies may include alpelisib as well as CDK4/6 inhibitors. In some instances, therapies may be target HER2 and include, for example, antibody drug conjugates (ADC).

In one or more examples, the targeted therapy may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for determining a receptor status of an individual may be used in treating a disease (e.g., a cancer) in a subject. For example, in response to determining a receptor status of an individual using any of the methods disclosed herein, an effective amount of an anti-cancer therapy or anti-cancer treatment may be administered to the subject. In some instances, only one anti-cancer therapy or anti-cancer treatment may be administered to the subject, while in other instances, one or more anti-cancer therapy or anti-cancer treatment may be administered to the subject.

In some instances, the disclosed methods for determining a receptor status of an individual may be used for monitoring disease progression or recurrence (e.g., cancer or tumor progression or recurrence) in a subject. For example, in some instances, the methods may be used to determine a receptor status of an individual based on a first sample obtained from the subject at a first time point, and used to determine a receptor status of the individual based on a second sample obtained from the subject at a second time point, where comparison of the first determination of the receptor status and the second determination of the receptor status allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of the receptor status.

In some instances, the value of determining a receptor status of an individual using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining a receptor status of an individual may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining a receptor status of an individual as part of a genomic profiling process (or inclusion of the output from the disclosed methods for determining a receptor status of an individual as part of the genomic profile of the subject) can improve the validity of, e.g., disease detection calls and treatment decisions, made on the basis of the genomic profile by, for example, independently confirming the presence of a receptor status in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a breast cancer.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, hormone driven cancers such as breast cancer and prostate cancer. In some instances, the cancers may include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM 333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB 351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™M series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde-or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM 1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule.

For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 103, 104, 105 or more than 105 molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1e−6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (See, e.g., SNVMix-Bioinformatics. 2010 March 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for determining a receptor status based on a sample from an individual. The systems may comprise, e.g., one or more processors, and a memory unit communicatively coupled to the one or more processors and configured to store instructions that, when executed by the one or more processors, cause the system to: receive, using one or more processors, training data comprising training values for a plurality of training input features associated with a receptor gene status in a plurality of training samples; train, using the one or more processors, a statistical model based on the training data; obtain, using the one or more processors, a set of input features associated with the receptor gene status, wherein obtaining the set of input features comprises omitting input features with a low prevalence and high correlation to the statistical model; receive, using one or more processors, sequence read data associated with a sample from an individual; determine, using the one or more processors, values for one or more input features associated with the receptor gene status based on the sequence read data, wherein the one or more input features to be evaluated are specified based on a sample type of the sample; input, using the one or more processors, the values for the one or more input features into the trained statistical model; and predict using the one or more processors, the receptor gene status of the individual based on an output of the trained statistical model.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for determining a receptor status of an individual in any of a variety of samples as described herein (e.g., a tissue sample, biopsy sample, hematological sample, or liquid biopsy sample derived from the subject).

In some instances, the plurality of gene loci for which sequencing data is processed to determine a receptor status may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a receptor status is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

FIG. 7 illustrates an example of a computing device or system in accordance with one embodiment. Device 700 can be a host computer connected to a network. Device 700 can be a client computer or a server. As shown in FIG. 7, device 700 can be any suitable type of microprocessor-based device, such as a personal computer, workstation, server or handheld computing device (portable electronic device) such as a phone or tablet. The device can include, for example, one or more processor(s) 710, input devices 720, output devices 730, memory or storage devices 740, communication devices 760, and nucleic acid sequencers 770. Software 750 residing in memory or storage device 740 may comprise, e.g., an operating system as well as software for executing the methods described herein. Input device 720 and output device 730 can generally correspond to those described herein, and can either be connectable or integrated with the computer.

Input device 720 can be any suitable device that provides input, such as a touch screen, keyboard or keypad, mouse, or voice-recognition device. Output device 730 can be any suitable device that provides output, such as a touch screen, haptics device, or speaker.

Storage 740 can be any suitable device that provides storage (e.g., an electrical, magnetic or optical memory including a RAM (volatile and non-volatile), cache, hard drive, or removable storage disk). Communication device 760 can include any suitable device capable of transmitting and receiving signals over a network, such as a network interface chip or device. The components of the computer can be connected in any suitable manner, such as via a wired media (e.g., a physical system bus 780, Ethernet connection, or any other wire transfer technology) or wirelessly (e.g., Bluetooth®, Wi-Fi®, or any other wireless technology).

Software module 750, which can be stored as executable instructions in storage 740 and executed by processor(s) 710, can include, for example, an operating system and/or the processes that embody the functionality of the methods of the present disclosure (e.g., as embodied in the devices as described herein).

Software module 750 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 740, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 750 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

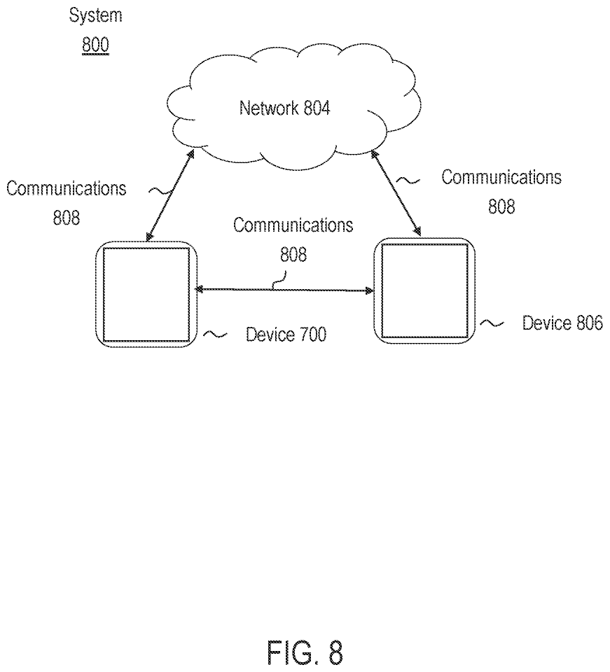

Device 700 may be connected to a network (e.g., network 804, as shown in FIG. 8 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 700 can be implemented using any operating system, e.g., an operating system suitable for operating on the network. Software module 750 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 710.

Device 700 can further include a sequencer 770, which can be any suitable nucleic acid sequencing instrument.

FIG. 8 illustrates an example of a computing system in accordance with one embodiment. In system 800, device 700 (e.g., as described above and illustrated in FIG. 7) is connected to network 804, which is also connected to device 806. In some embodiments, device 806 is a sequencer. Exemplary sequencers can include, without limitation, Roche/454's Genome Sequencer (GS) FLX System, Illumina/Solexa's Genome Analyzer (GA), Illumina's HiSeq®2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 700 and 806 may communicate, e.g., using suitable communication interfaces via network 804, such as a Local Area Network (LAN), Virtual Private Network (VPN), or the Internet. In some embodiments, network 804 can be, for example, the Internet, an intranet, a virtual private network, a cloud network, a wired network, or a wireless network. Devices 700 and 806 may communicate, in part or in whole, via wireless or hardwired communications, such as Ethernet, IEEE 802.11b wireless, or the like. Additionally, devices 700 and 806 may communicate, e.g., using suitable communication interfaces, via a second network, such as a mobile/cellular network. Communication between devices 700 and 806 may further include or communicate with various servers such as a mail server, mobile server, media server, telephone server, and the like. In some embodiments, Devices 700 and 806 can communicate directly (instead of, or in addition to, communicating via network 804), e.g., via wireless or hardwired communications, such as Ethernet, IEEE 802.11b wireless, or the like. In some embodiments, devices 700 and 806 communicate via communications 808, which can be a direct connection or can occur via a network (e.g., network 804).

One or all of devices 700 and 806 generally include logic (e.g., http web server logic) or are programmed to format data, accessed from local or remote databases or other sources of data and content, for providing and/or receiving information via network 804 according to various examples described herein.

### EXAMPLES

This section provides a non-limiting example of training a machine learning model in accordance with embodiments of the present disclosure. In one or more examples, embodiments of the present disclosure may be used to train a statistical model to determine a receptor status.

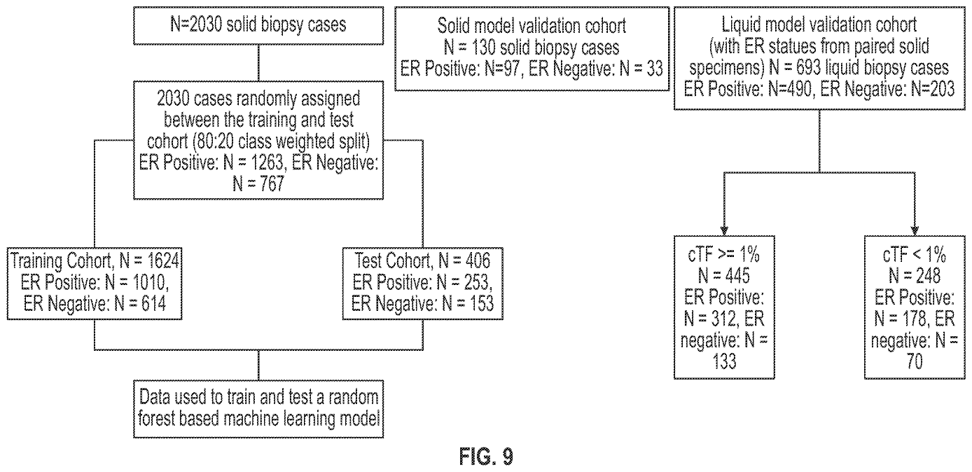

FIG. 9 illustrates an exemplary process for training a statistical model in accordance with embodiments of the present disclosure. As shown in the figure, a statistical model for determining an ER status for an individual was trained and validated using 2,030 solid biopsy samples. This cohort of 2,030 samples was derived from solid biopsy specimens of cancer patients, which were previously sequenced in an accredited laboratory. To create a stringent high-quality cohort of cases, the 2,030 solid biopsy samples were confirmed to pass tumor purity, sample quality, and copy number noise quality control criteria. The resulting quality-controlled dataset of 2,030 cases underwent an 80:20 class-weighted random split to yield 1,624 cases for the training cohort and 406 cases for the testing cohort as shown in the figure.

According to this example, the statistical model was trained using a random forest-based machine learning algorithm based on a cohort of 1,624 breast cancer solid tumor cases (e.g., 1,010 ER positive samples and 614 ER negative samples) using one or more training input feature values, e.g., one or more genomic alteration features, one or more complex mutation signature features, one or more chromosomal instability features, one or more clinicopathological features, one or more clinical features, one or more additional features, or a combination thereof. The trained statistical model was tested on an independent cohort of 406 cases (e.g., 253 ER positive samples and 153 ER negative samples).

Based on the dataset of 2,030 solid biopsy samples, the system produced two models: one model to predict ER status in solid biopsy derived cases corresponding to a first set of input features and another model for liquid biopsy derived cases corresponding to a second set of input features. As shown in the figure, independent validation cohorts were chosen based on whether the model was trained to predict ER status for solid cases or liquid cases.

According to this example, a binary classifier was built using the random forest algorithm, on a training cohort of 1,624 ER positive and 614 ER negative cases. Separate models were built for to determine the ER status for solid biopsy samples and for liquid biopsy samples. Each of these models included different features. For example, the model for solid samples included 194 features while the liquid model included 15 features. The model parameters, of the classifier models, including the number of trees grown and size of the random feature subset considered at each split, were tuned by a Cartesian hyperparameter grid search, to maximize AUC (ROC), with a scalable machine learning platform (e.g., H20.ai v3.28.0.4, in R v4.0.3). To adjust for class imbalance between ER positive and ER negative cases during model training, a stratified sampling methodology was used and an equal number of cases were sampled from the ER positive cases and ER negative cases, equal to 60% of the total ER negative cases in the training cohort. Prediction performance of the model was estimated on the training cohort by 10-fold cross validation and an independent test cohort of 406 solid samples (e.g., 253 ER positive samples and 153 ER negative samples) were also used to evaluate the performance of classifier model. The solid and liquid models were then correspondingly applied to the validation cohorts of 130 solid biopsy cases (e.g., 97 ER positive samples and 33 ER negative samples) and 693 liquid biopsy cases, respectively. For the liquid biopsy cases, the ER statuses were determined based on paired solid biopsy specimens.

For example, a validation cohort of 130 solid biopsy samples (e.g., 97 ER positive samples and 33 ER negative samples) was used to validate the solid model. Based on this example, the system achieved an accuracy of 83% with respect to determining the ER status of the solid biopsy validation samples.

A validation cohort of 693 liquid biopsy samples was used to validate the liquid model. The ER status information was determined for the cohort of 693 liquid biopsy samples based on a paired solid biopsy specimen (e.g., 490 ER positive samples and 203 ER negative samples). For example, the ER status of the paired solid biopsy sample was analyzed to determine the ER receptor status for validation.

As shown in FIG. 9, the liquid samples were further analyzed based on the circulating tumor fraction (cTF) of the sample. For example, as shown in the figure, 445 liquid biopsy samples had a cTF above 1% (e.g., 312 ER positive samples and 133 ER negative samples). Based on this example, the system achieved an accuracy of 77% based on the liquid biopsy validation samples with a cTF above 1%. As shown in the figure, 248 liquid biopsy samples had a cTF below 1% (e.g., 178 ER positive samples and 70 ER negative samples). Based on this example, the system achieved an accuracy of 69% based on liquid biopsy validation samples with a cTF below 1%. This demonstrates that this exemplary trained model performs near a clinically meaningful level (e.g., around 75%) for liquid biopsy samples even for low levels of cTF (e.g., below 1%). Accordingly, examples in accordance with embodiments of this disclosure can provide clinically meaningful receptor predictions for both solid and liquid samples. Further in the case for liquid samples, examples in accordance with embodiments of this disclosure can provide clinically meaningful receptor predictions for samples with low levels of cTF.

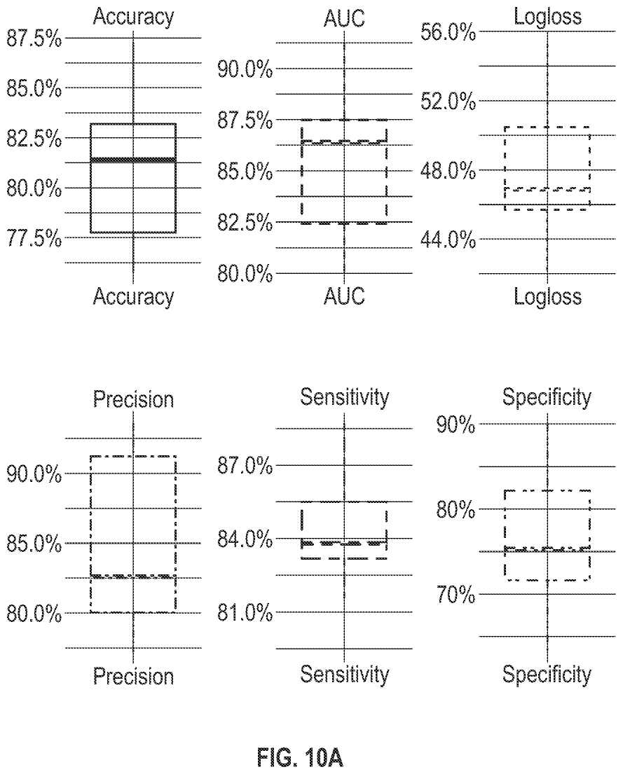

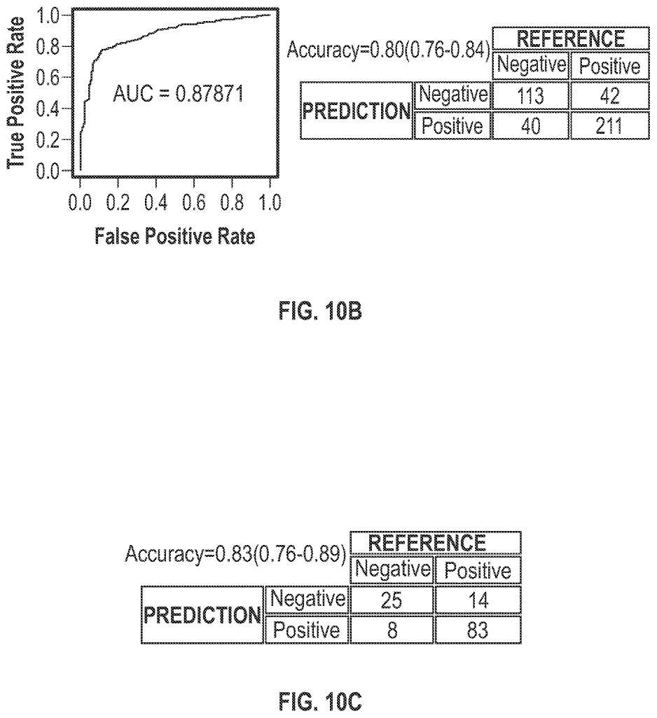

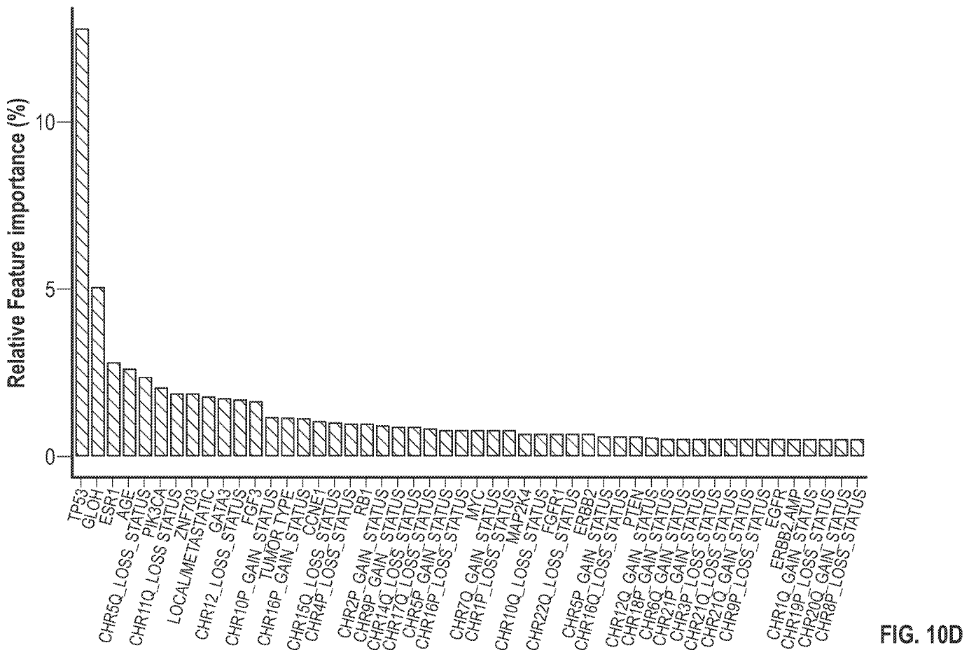

According to one example, a binary classifier using a random forest algorithm on a training cohort of 1,010 ER+ and 614 ER− cases was built to determine the ER receptor status for solid tissue samples. The binary classifier parameters, including number of trees grown and size of the random feature subset were considered at each split, were tuned by a cartesian hyperparameter grid search, to maximize AUC (ROC), with a scalable machine learning platform. To adjust for class imbalance between ER+ and ER− cases during model training, a stratified sampling methodology was used and an equal number of cases were sampled from the ER+ cases and ER− cases, equal to 60% of the total ER− cases in the training cohort. Prediction performance of the model was estimated on the training cohort by 10-fold cross validation and an independent test cohort of 253 ER+ cases and 153 ER− cases was also used to evaluate the performance of classification. Performance metrics are described in FIGS. 10A-10C.

FIG. 10A illustrates the 10-fold cross validation metrics of the random forest solid model on the solid training dataset. FIG. 10B illustrates an exemplary performance metrics of the random forest solid model on the solid test dataset. As shown in the figure, the accuracy of the model on the test dataset is about 80%. FIG. 10C illustrates an exemplary performance metrics of the random forest solid model on the solid validation dataset. As shown in the figure, the accuracy of the model on the test dataset is about 83%. FIG. 10D illustrates the relative feature importance of fifty input features out of an input feature set comprising 194 features. The features included genomic features, complex mutational signatures, chromosomal instability, and clinicopathological features, as described above. In some examples, after fitting the model and studying feature importance, the system can retrospectively select which features to keep and estimate the percentage of features to be kept and removed.

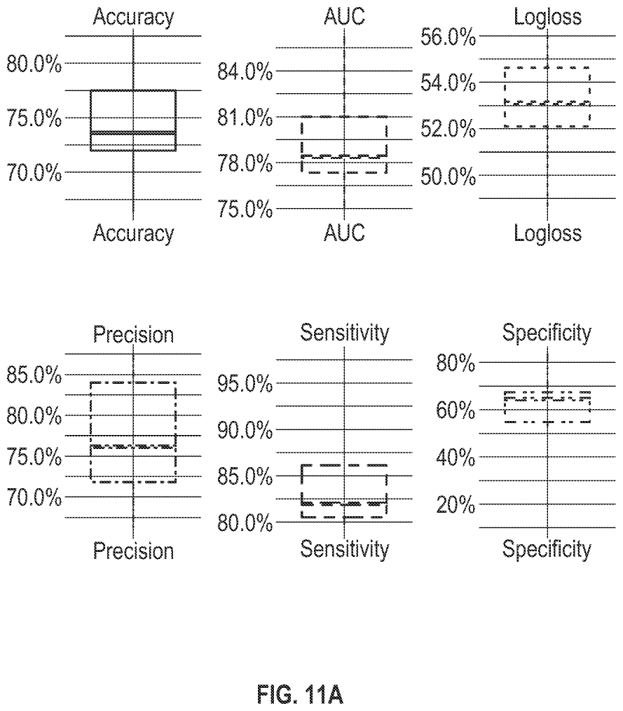

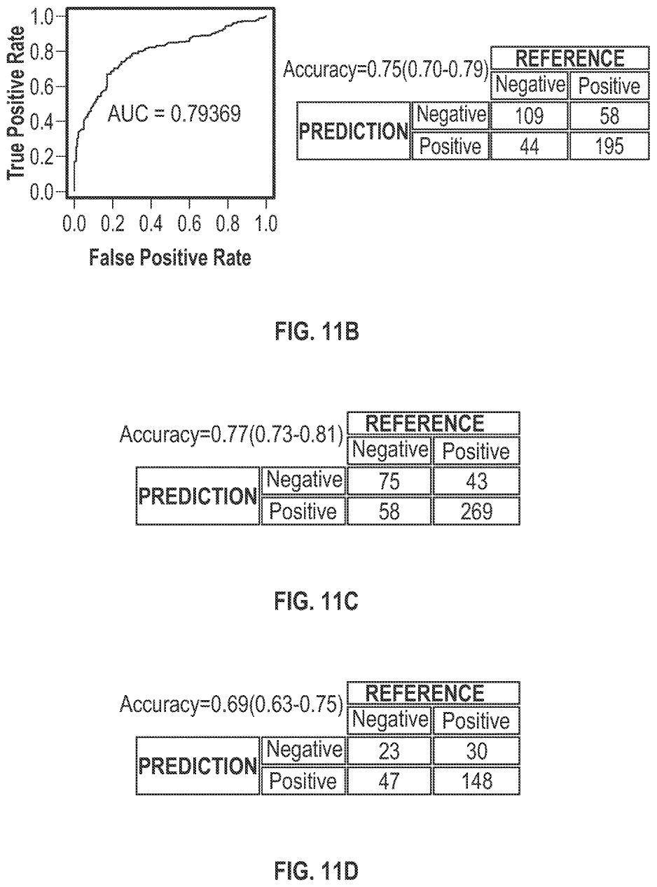

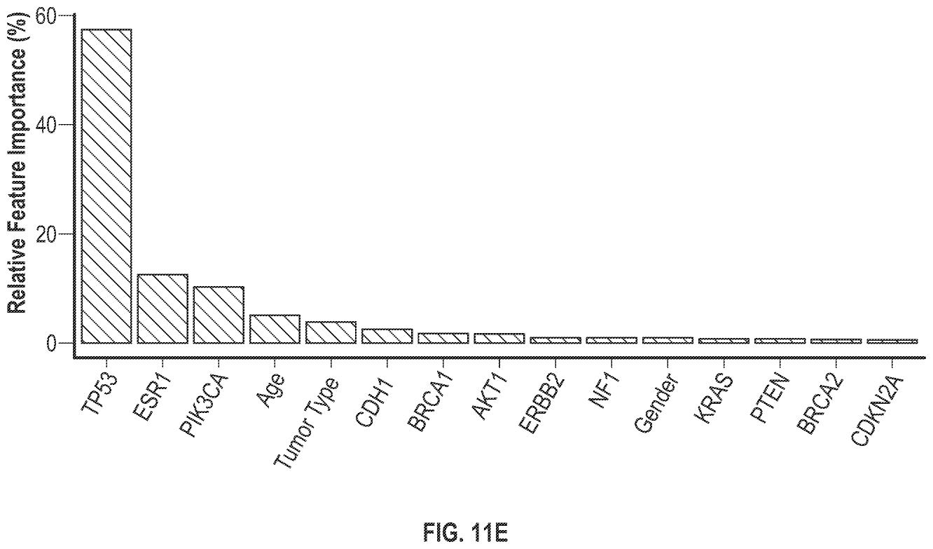

FIG. 11A illustrates the 10-fold cross validation metrics of the random forest liquid model on the liquid training dataset. FIG. 11B illustrates exemplary performance metrics of the random forest liquid model on the liquid test dataset. As shown in the figure, the accuracy of the model on the test dataset is about 75%. FIG. 11C illustrates exemplary performance metrics of the random forest liquid model on the liquid validation dataset with a cTF greater than one percent. As shown in the figure, the accuracy of the model on the test dataset is about 77%. FIG. 11D illustrates exemplary performance metrics of the random forest liquid model on the liquid validation dataset with a cTF less than one percent. As shown in the figure, the accuracy of the model on the validation dataset is about 69%. FIG. 11E illustrates the relative feature importance of the fifteen input features corresponding to the set of input features for the exemplary liquid model.

### EXEMPLARY IMPLEMENTATIONS

Exemplary implementations of the methods and systems described herein include:
1. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of reads that represent the captured nucleic acid molecules;
   receiving, at one or more processors, sequence read data for the plurality of reads;
   receiving, using one or more processors, training data comprising training values for a plurality of training input features associated with a receptor gene status in a plurality of training samples;
   training, using the one or more processors, a statistical model based on the training data;
   obtaining, using the one or more processors, a set of input features associated with the receptor gene status, wherein obtaining the set of input features comprises omitting input features with a low prevalence and high correlation to the statistical model;
   determining, using the one or more processors, values for one or more input features associated with the receptor gene status based on the sequence read data, wherein the one or more input features to be evaluated are specified based on a sample type of the sample;
   inputting, using the one or more processors, the values for the one or more input features into the trained statistical model; and
   predicting, using the one or more processors, the receptor gene status of the subject based on an output of the trained statistical model.
2. The method of clause 1, wherein the receptor gene status corresponds to an estrogen receptor status, a progesterone receptor status, an androgen receptor status, or a human epidermal growth factor receptor 2 status.
3. The method of any of clauses 1 to 2, wherein the receptor gene status comprises a hormone receptor status.
4. The method of any of clauses 1 to 3, further comprising applying the trained statistical model to the values for the one or more input features to obtain an output indicative of the receptor gene status.
5. The method of any of clauses 1 to 4, wherein the sample type is indicative of a solid sample and the set of input features comprises genomic alteration features, complex mutational signatures, chromosomal instability features, clinicopathological features, clinical features, additional features, or a combination thereof.
6. The method of clause 5, wherein the genomic alteration features comprise one or more genomic alterations and a type of alteration associated with the one or more genomic alterations.
7. The method of any of clauses 1 to 4, wherein the sample type is indicative of a liquid sample and the set of input features comprises genomic alteration features, clinicopathological features, clinical features, tumor fraction features, fragmentomic features, additional features, or a combination thereof.
8. The method of clause 5, wherein the genomic alteration features comprise one or more genomic alterations and a type of alteration associated with the one or more genomic alterations.
9. The method of any one of clauses 1 to 8, wherein the subject is suspected of having or is determined to have cancer.
10. The method of clause 9, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
11. The method of clause 9, wherein the cancer comprises breast cancer (HER2 overexpressed/amplified), breast cancer (HER2+), breast cancer (HR+, HER2−), and prostate cancer.
12. The method of clause 11, further comprising treating the subject with an anti-cancer therapy.
13. The method of clause 12, wherein the anti-cancer therapy comprises a targeted anti-cancer therapy.
14. The method of clause 13, wherein the targeted anti-cancer therapy comprises alpelisib (Piqray), CDK4/6 inhibitors, or any combination thereof.
15. The method of any of clauses 1 to 14, further comprising obtaining the sample from the subject.
16. The method of any of clauses 1 to 15, wherein the sample comprises a tissue biopsy sample, a liquid biopsy sample, or a normal control.
17. The method of any of clauses 1 to 16, wherein the set of features differs between a tissue biopsy sample and a liquid biopsy.
18. The method of clause 16, wherein the sample is a liquid biopsy sample and comprises blood, plasma, cerebrospinal fluid, sputum, stool, urine, or saliva.
19. The method of clause 16, wherein the sample is a liquid biopsy sample and comprises circulating tumor cells (CTCs), cell-free DNA (cfDNA), circulating tumor DNA (ctDNA), or any combination thereof.
20. The method of any one of clauses 1 to 19, wherein the plurality of nucleic acid molecules comprises a mixture of tumor nucleic acid molecules and non-tumor nucleic acid molecules.
21. The method of clause 20, wherein the tumor nucleic acid molecules are derived from a tumor portion of a heterogeneous tissue biopsy sample, and the non-tumor nucleic acid molecules are derived from a normal portion of the heterogeneous tissue biopsy sample.
22. The method of clause 20, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
23. The method of any one of clauses 1 to 22, wherein the one or more adapters comprise amplification primers, flow cell adaptor sequences, substrate adapter sequences, or sample index sequences.
24. The method of any one of clauses 1 to 23, wherein the captured nucleic acid molecules are captured from the amplified nucleic acid molecules by hybridization to one or more bait molecules.
25. The method of clause 24, wherein the one or more bait molecules comprise one or more nucleic acid molecules, each comprising a region that is complementary to a region of a captured nucleic acid molecule.
26. The method of any one of clauses 1 to 25, wherein amplifying nucleic acid molecules comprises performing a polymerase chain reaction (PCR) amplification technique, a non-PCR amplification technique, or an isothermal amplification technique.
27. The method of any one of clauses 1 to 26, wherein the sequencing comprises use of a massively parallel sequencing (MPS) technique, whole genome sequencing (WGS), whole exome sequencing, targeted sequencing, direct sequencing, or Sanger sequencing technique.
28. The method of clause 27, wherein the sequencing comprises massively parallel sequencing, and the massively parallel sequencing technique comprises next generation sequencing (NGS).
29. The method of any one of clauses 1 to 28, wherein the sequencer comprises a next generation sequencer.
30. The method of any one of clauses 1 to 29, wherein one or more of the plurality of sequencing reads overlap one or more gene loci within one or more subgenomic intervals in the sample.
31. The method of clause 30, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci. 32. The method of clause 30 or clause 31, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
33. The method of clause 30 or clause 31, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-16, IL-6, IL-6R, JAK1, JAK2, JAK3, KIT, KRAS, MEK, MET, MSI-H, mTOR, PARP, PD-1, PDGFR, PDGFRa, PDGFRØ, PD-L1, PI3K8, PIGF, PTCH, RAF, RANKL, RET, ROS1, SLAMF7, VEGF, VEGFA, VEGFB, or any combination thereof.
34. The method of any one of clauses 1 to 33, further comprising generating, by the one or more processors, a report indicating a receptor gene status of the sample.
35. The method of clause 34, further comprising transmitting the report to a healthcare provider via a computer network or a peer-to-peer connection.
36. A method comprising:
   receiving, using one or more processors, training data comprising training values for a plurality of training input features associated with a receptor gene status in a plurality of training samples;
   training, using the one or more processors, a statistical model based on the training data;
   obtaining, using the one or more processors, a set of input features associated with the receptor gene status and a sample type, wherein obtaining the set of input features comprises omitting input features with a low prevalence and high correlation to the statistical model;
      receiving, using one or more processors, sequence read data associated with a sample from an individual;
      determining, using the one or more processors, values for one or more input features associated with the receptor gene status based on the sequence read data, wherein the one or more input features to be evaluated are specified based on a sample type of the sample;
      inputting, using the one or more processors, the values for the one or more input features into the trained statistical model; and
      predicting, using the one or more processors, the receptor gene status of the individual based on an output of the trained statistical model.
37. The method of clause 36, wherein the receptor gene status corresponds to an estrogen receptor status, a progesterone receptor status, an androgen receptor status, or a human epidermal growth factor receptor 2 status.
38. The method of any of clauses 36 to 37, further comprising applying the trained machine learning model to the values for the one or more input features to obtain an output indicative of the receptor gene status.
39. The method any of clauses 36 to 38, wherein the sequence read data for the individual is derived from a solid sample.
40. The method of clause 39, wherein the sequence read data for the individual is derived from a biopsy sample.
41. The method of clause 39, wherein the set of input features comprises genomic alteration features, complex mutational signatures, chromosomal instability features, clinicopathological features, clinical features, additional features, or a combination thereof.
42. The method any of clauses 39 to 41, wherein the one or more input features are associated with one or more genomic alteration features.
43. The method of clause 42, wherein the one or more genomic alteration features comprise a presence of a predetermined short variant, an absence of a predetermined short variant, a copy number alteration, a rearrangement alteration, an amplification, a deletion, or a combination thereof.
44. The method of clause 43, wherein the predetermined short variant comprises a point mutation, an insertion, or a deletion.
45. The method of any of clauses 42 to 44, wherein the one or more genomic alteration features comprise CCNE1, EGFR, ERBB2, ERBB2 Amplification, ESR1, FGF3, FGFR1, GATA3, MAP2K4, MYC, PIK3CA, PTEN, RB1, TP53, ZNF703, or a combination thereof.
46. The method of any of clauses 42 to 44, wherein the one or more genomic alteration features comprise BRCA1, TP53, MYC, ERBB2, ERBB2 Amplification, ERBB2 Short Variant, PIK3CA, ESR1, TBX3, CDH1, ZNF703, GATA3, FGF3, CCNE1, RB1, FGFR1, PTEN, EGFR, MAP2K4, or a combination thereof.
47. The method of any of clauses 42 to 44, wherein the one or more genomic alteration features comprise AKT1, AKT2, AKT3, APC, AR, ARFRP1, ARID1A, ATM, AURKA, BAP1, BCL2L1, BCL2L2, BRAF, BRCA1, BRCA2, BRD4, CBFB, CCND3, CCNE1, CDH1, CDK12, CDK4, CDK6, CDKN2A, CDKN2B, CHEK2, CREBBP, CTNNA1, DNMT3A, EGFR, EMSY, EP300, ERBB2, ERBB2 Amplification, ERBB2 Short Variant, ERBB3, ESR1, FANCA, FAS, FBXW7, FGF10, FGF12, FGF3, FGF6, FGFR1, FGFR2, FGFR4, GATA3, GNAS, HGF, IGF1R, IKBKE, IRS2, JAK2, KDM5A, KDM6A, KDR, KEL, KIT, KMT2D, KRAS, LYN, MAP2K4, MAP3K1, MCL1, MDM2, MDM4, MSH6, MUTYH, MYC, MYCL, NF1, NFKBIA, NKX2 1, NOTCH1, NOTCH2, NOTCH3,, TRK1, PALB2, PBRM1, PDCD1LG2, PDGFRA, PIK3C2B, PIK3CA, PIK3CB, PIK3R1, PRKCI, PTEN, RAF1, RB1, RET, RICTOR, ROS1, RPTOR, SETD2, SF3B1, SMAD4, SMARCA4, SOX2, SPEN, SRC, STK11, TBX3, TERC, TET2, TP53, TSC1, VEGFA, ZNF217, ZNF703, or a combination thereof.
48. The method of any of clauses 39 to 47, wherein the one or more input features are associated with one or more complex mutational signatures.
49. The method of clause 48, wherein the one or more complex mutational signatures comprise a genome-wide loss of heterozygosity (gLOH) quantification, trinucleotide signatures, insertion signatures, deletion signatures, copy number signatures, or a combination thereof.
50. The method of any of clauses 39 to 49, wherein the one or more input features are associated with one or more chromosomal instability features.
51. The method of clause 50, wherein the one or more chromosomal instability features is indicative of aneuploidy.
52. The method of clause 50, wherein the one or more chromosomal instability features is indicative of chromosome gain, chromosome not gain, chromosome loss, chromosome not loss, or a combination thereof.
53. The method of clause 50, wherein the one or more chromosomal instability features comprises a total aneuploidy count.
54. The method of clause 50, wherein the one or more chromosomal instability features comprise chromosome 1q loss status, chromosome 1p loss status, chromosome 3p loss status, chromosome 4p loss status, chromosome 5q loss status, chromosome 9p loss status, chromosome 10q loss status, chromosome 11q loss status, chromosome 12q loss status, chromosome 14q loss status, chromosome 15q loss status, chromosome 16p loss status, chromosome 16q loss status, chromosome 17q loss status, chromosome 19p loss status, chromosome 21q loss status, chromosome 22q loss status, chromosome 1q gain status, chromosome 2p gain status, chromosome 5p gain status, chromosome 5q gain status, chromosome 6q gain status, chromosome 7q gain status, chromosome 9p gain status, chromosome 10p gain status, chromosome 12q gain status, chromosome 16p gain status, chromosome 18p gain status, chromosome 20q gain status chromosome 21p gain status, chromosome 21q gain status.
55. The method of clause 50, wherein the one or more chromosomal instability features comprise chromosome 1p gain status, chromosome 1q gain status, chromosome2p gain status, chromosome 2q gain status, chromosome 3p gain status, chromosome 3q gain status, chromosome 4p gain status, chromosome 4q gain status, chromosome 5p gain status, chromosome 5q gain status, chromosome 6p gain status, chromosome 6q gain status, chromosome 7p gain status, chromosome 7q gain status, chromosome 8p gain status, chromosome 8q gain status, chromosome 9p gain status, chromosome 9q gain status, chromosome 10p gain status, chromosome 10q gain status, chromosome 11p gain status, chromosome 11q gain status, chromosome 12p gain status, chromosome 12q gain status, chromosome 13q gain status, chromosome 14q gain status, chromosome 15q gain status, chromosome 16p gain status, chromosome 16q gain status, chromosome 17p gain status, chromosome 17q gain status, chromosome 18p gain status, chromosome 18q gain status, chromosome 19p gain status, chromosome 19q gain status, chromosome 20p gain status, chromosome 20q gain status, chromosome 21p gain status, chromosome 21q gain status, chromosome 22q gain status, chromosome 1p loss status, chromosome 2p loss status, chromosome 2q loss status, chromosome 3p loss status, chromosome 3q loss status, chromosome 4p loss status, chromosome 4q loss status, chromosome 5p loss status, chromosome 5q loss status, chromosome 6p loss status, chromosome 6q loss status, chromosome 7p loss status, chromosome 7q loss status, chromosome 8p loss status, chromosome 8q loss status, chromosome 9p loss status, chromosome 9q loss status, chromosome 10p loss status, chromosome 10q loss status, chromosome 11p loss status, chromosome 11q loss status, chromosome 12p loss status, chromosome 12q loss status, chromosome 13q loss status, chromosome 14q loss status, chromosome 15q loss status, chromosome 16p loss status, chromosome 16q loss status, chromosome 17p loss status, chromosome 17q loss status, chromosome 18p loss status, chromosome 18q loss status, chromosome 19p loss status, chromosome 19q loss status, chromosome 20p loss status, chromosome 20q loss status, chromosome 21p loss status, chromosome 21q loss status, chromosome 22q loss status.
56. The method of any of clauses 39 to 55, wherein the one or more input features are associated with one or more clinicopathological features.
57. The method of clause 56, wherein the one or more clinicopathological features comprise an age of the individual, a sex of the individual, a disease diagnosis of the individual, a tumor status of the individual, a tumor type, an anatomical sub-classification of a tumor, or a combination thereof.
58. The method of any of clauses 39 to 57, wherein the one or more input features are associated with one or more clinical features.
59. The method of clause 58, wherein the one or more clinical features comprise an ancestry of the individual, a family history of the individual, a stage of the disease, one or more laboratory-based test results, one or more lifestyle habits, a diabetes status, an obesity status, an indication of body vitamin levels, or a combination thereof.
60. The method of any of clauses 39 to 59, wherein the one or more input features are associated with a tumor mutational burden.
61. The method of any of clauses 39 to 60, wherein the one or more input features are associated with a germline status.
62. The method of any of clauses 39 to 61, wherein the one or more input features are associated with a methylation signature, a mRNA expression level, a miRNA expression level, proteomics, COSMIC mutation signatures, immunohistochemical markers, genetic predispositions, cell adhesion biomarkers, saliva based biomarkers, enzyme based biomarkers, a diagnostic image, a homologous repair deficiency (HRD) signature, or a combination thereof.
63. The method of any of clauses 36 to 62, wherein sequence read data derived from a liquid sample is associated with a first set of one or more input features and sequence read data derived from a solid sample is associated with a second set of one or more input features.
64. The method any of clauses 36 to 38 and 63, wherein the sequence read data for the individual is derived from a liquid sample.
65. The method of clause 64, wherein the sequence read data for the individual is derived from a liquid biopsy sample.
66. The method of any of clauses 64 to 65, wherein the set of input features comprises genomic alteration features, clinicopathological features, clinical features, tumor fraction features, fragmentomic features, additional features, or a combination thereof.
67. The method any of clauses 64 to 66, wherein the one or more input features are associated with one or more genomic alteration features.
68. The method of clause 67, wherein the one or more genomic alteration features comprise a presence of a predetermined short variant, an absence of a predetermined short variant, a rearrangement alteration, an insertion, a deletion, or a combination thereof.
69. The method of any of clauses 67 to 68, wherein the one or more genomic alteration features comprise TP53, ESR1, PIK3CA, or a combination thereof.
70. The method of clause 67 to 68, wherein the one or more genomic alteration features comprise CDH1, BRCA1, AKT1, BRCA2, CDKN2A, ERBB2, KRAS, NF1, PTEN, or a combination thereof.
71. The method of any of clauses 64 to 70, wherein the one or more input features are associated with one or more clinicopathological features.
72. The method of clause 71, wherein the one or more clinicopathological features comprise an age of the individual, a sex of the individual, a disease diagnosis of the individual, a tumor type, or a combination thereof.
73. The method of any of clauses 64 to 72, wherein the one or more input features are associated with one or more clinical features.
74. The method of clause 73, wherein the one or more clinical features comprise an ancestry of the individual, a stage of the disease, one or more laboratory-based test results, one or more lifestyle habits, a diabetes status, an obesity status, an indication of body vitamin levels, or a combination thereof.
75. The method of any of clauses 64 to 74, wherein the one or more input features are associated with a tumor mutational burden.
76. The method of any of clauses 64 to 75, wherein the one or more input features are associated with trinucleotide signatures, insertion signatures, deletion signatures, copy number signatures, or a combination thereof.
77. The method of any of clauses 64 to 76, wherein the one or more input features are associated with the HRD signature.
78. The method of any of clauses 64 to 77, wherein the one or more input features are associated with one or more fragmentomic features, the one or more fragmentomic features associated with a plurality of reads obtained from the sequence read data.
79. The method of clause 78, wherein the one or more fragmentomic features comprise lengths of the plurality reads, regions associated with the plurality of reads, or a combination thereof.
80. The method of clause 78 to 79, wherein the one or more fragmentomic features comprise an amount of a fragment having a specified length, a mean fragment length of the plurality of reads, a median fragment length of the plurality of reads, an interquartile range of fragment lengths of the plurality of reads, a distribution of fragment lengths of the plurality of reads, one or more peaks of fragment lengths for the plurality of reads, a fragment length for the plurality of reads, a start position of a fragment for the plurality of reads, and an end position of a fragment for the plurality of reads.
81. The method of any of clauses 64 to 80, wherein the one or more input features are associated with an estimated tumor fraction.
82. The method of any of clauses 64 to 81, wherein the one or more input features are associated with a methylation signature, a mRNA expression level, a miRNA expression level, proteomics, COSMIC mutation signatures, immunohistochemical markers, genetic predispositions, cell adhesion biomarkers, saliva based biomarkers, enzyme based biomarkers, a diagnostic image, or a combination thereof.
83. The method of any of clauses 36 to 82, wherein the output of the trained statistical model is indicative of a receptor status.
84. The method of clause 83, wherein the output of the trained statistical model comprises a first score indicative of a probability of a positive receptor gene status.
85. The method of any of clauses 83 to 84, wherein the output of the trained statistical model comprises a second score indicative of a probability of a negative receptor gene status.
86. The method of any of clauses 36 to 85, wherein the training set of input features associated with the training values for the input features is different from the values for one or more input features input into the trained statistical model.
87. The method of the clause 36 to 86, wherein the training data further comprises receptor gene statuses corresponding to each of the plurality of training samples.
88. The method of any of clauses 86 to 87, further comprising:
   determining, using the one or more processors, weights associated with the training values for the plurality of training input features based on the training;
   filtering, using the one or more processors, the plurality of training input features based on the weights; and
   determining, using the one or more processors, the set of input features based on the filtered plurality of training input features, wherein filtering the plurality of training input features comprises removing low prevalence training values, highly correlated training values, or a combination thereof.
89. The method of clause 88, further comprising weighting, using the one or more processors, the training values for the one or more input features based on the weights, wherein predicting the receptor gene status is based on the weighted values for the one or more input features.
90. The method of any of clauses 36 to 89, wherein the receptor gene status comprises a hormone receptor status.
91. The method of any of clauses 36 to 90, wherein the trained statistical model is a trained machine learning model, a part of a machine learning process, or a combination thereof.
92. The method of any of clauses 36 to 91, wherein the trained statistical model includes an artificial intelligence learning model.
93. The method of any of clauses 36 to 92, wherein the trained statistical model comprises a random forest model.
94. The method of any of clauses 36 to 93, wherein the trained statistical model comprises at least one of an extreme gradient boosting model, a logistic regression model, an elastic net model, a ridge regression model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.
95. The method of any of claims 36 to 94, further comprising:
   inputting, using the one or more processors, a diagnostic image taken of the individual into a second statistical model; and
   determining, using the one or more processors, a third score indicative of a tumor classification based on the diagnostic image,
   wherein predicting the receptor gene status is further based on the third score.
96. The method of clause 95, wherein the second statistical model is at least one of a deep learning model, a convolutional neural network model, a recurrent neural network model, and an auto-encoder model.
97. The method of any of clauses 95 to 96, further comprising training the second statistical model, wherein the second statistical model is trained by:
   receiving, using the one or more processors, training data including a plurality of training diagnostic images; and
   training, using the one or more processors, the second statistical model based on the training data.
98. The method of any of clauses 95 to 97, wherein the diagnostic image comprises at least one of a histopathological image, a radiological image, a magnetic resonance image, an ultrasound image, a X-ray image, a bone scan, a CT scan, a PET scan, or a combination thereof.
99. The method any of clauses 36 to 98, further comprising assigning, using the one or more processors, a therapy for the individual based on the predicted receptor gene status.
100. The method any of clauses 36 to 99, further comprising determining, using the one or more processors, a treatment decision for the individual based on the predicted receptor gene status.
101. The method any of clauses 36 to 100, further comprising administering, using the one or more processors, a treatment to the individual based on the predicted receptor gene status.
102. The method any of clauses 36 to 101, further comprising monitoring, using the one or more processors, a prognosis of the individual based on the predicted receptor gene status.
103. The method any of clauses 102, further comprising predicting, using the one or more processors, one or more clinical outcomes based on the predicted receptor gene status.
104. A method for diagnosing a disease, the method comprising:
   diagnosing that a subject has the disease based on a determination of a receptor gene status for a sample from the subject, wherein the receptor gene status is determined according to the method of any one of clauses 36 to 103.
105. A method of selecting an anti-cancer therapy, the method comprising:
   responsive to determining a receptor gene status for a sample from a subject, selecting an anti-cancer therapy for the subject, wherein the receptor gene status is determined according to the method of any one of clauses 36 to 103.
106. A method of treating a cancer in a subject, comprising:
   responsive to determining a receptor gene status for a sample from the subject, administering an effective amount of an anti-cancer therapy to the subject, wherein the receptor gene status is determined according to the method of any one of clauses 36 to 103.
107. A method for monitoring cancer progression or recurrence in a subject, the method comprising:
   determining a first receptor gene status in a first sample obtained from the subject at a first time point according to the method of any one of clauses 36 to 103;
   determining a second receptor gene status in a second sample obtained from the subject at a second time point; and comparing the first receptor gene status to the second receptor gene status, thereby monitoring the cancer progression or recurrence.
108. The method of clause 107, wherein the second receptor gene status for the second sample is determined according to the method of any one of clauses 36 to 103.
109. The method of any of clauses 107 to 108, further comprising selecting an anti-cancer therapy for the subject in response to the cancer progression.
110. The method of clauses 107 to 108, further comprising administering an anti-cancer therapy to the subject in response to the cancer progression.
111. The method of clause 107 to 108, further comprising adjusting an anti-cancer therapy for the subject in response to the cancer progression.
112. The method of any one of clauses 109 to 111, further comprising adjusting a dosage of the anti-cancer therapy or selecting a different anti-cancer therapy in response to the cancer progression.
113. The method of clause 112, further comprising administering the adjusted anti-cancer therapy to the subject.
114. The method of any one of clauses 107 to 113, wherein the first time point is before the subject has been administered an anti-cancer therapy, and wherein the second time point is after the subject has been administered the anti-cancer therapy.
115. The method of any one of clauses 107 to 114, wherein the subject has a cancer, is at risk of having a cancer, is being routine tested for cancer, or is suspected of having a cancer.
116. The method of any one of clauses 107 to 115, wherein the cancer is a solid tumor.
117. The method of any one of clauses 107 to 115, wherein the cancer is a breast cancer.
118. The method of any one of clauses 107 to 117, wherein the anti-cancer therapy comprises chemotherapy, radiation therapy, immunotherapy, a targeted therapy, or surgery.
119. The method of any one of clauses 36 to 103, further comprising determining, identifying, or applying the receptor gene status for the sample as a diagnostic value associated with the sample.
120. The method of any one of clauses 36 to 103, further comprising generating a genomic profile for the subject based on the determination of the receptor gene status.
121. The method of clause 120, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
122. The method of any of clauses 120 to 121, wherein the genomic profile for the subject further comprises results from a nucleic acid sequencing-based test.
123. The method of any of clauses 120 to 122, further comprising selecting an anti-cancer therapy, administering an anti-cancer therapy, or applying an anti-cancer therapy to the subject based on the generated genomic profile.
124. The method of any one of clauses 36 to 103, wherein the determination of receptor gene status for the sample is used in making suggested treatment decisions for the subject.
125. The method of any one of clauses 36 to 103, wherein the determination of the receptor gene status for the sample is used in applying or administering a treatment to the subject.
126. A system comprising:
   one or more processors; and
   a memory communicatively coupled to the one or more processors and configured to store instructions that, when executed by the one or more processors, cause the system to:
      receive, using one or more processors, training data comprising training values for a plurality of training input features associated with a receptor gene status in a plurality of training samples;
      train, using the one or more processors, a statistical model based on the training data;
      obtain, using the one or more processors, a set of input features associated with the receptor gene status, wherein obtaining the set of input features comprises omitting input features with a low prevalence and high correlation to the statistical model;
      receive, using one or more processors, sequence read data associated with a sample from an individual;
      determine, using the one or more processors, values for one or more input features associated with the receptor gene status based on the sequence read data, wherein the one or more input features to be evaluated are specified based on a sample type of the sample;
      input, using the one or more processors, the values for the one or more input features into the trained statistical model; and
      predict using the one or more processors, the receptor gene status of the individual based on an output of the trained statistical model.
127. A non-transitory computer-readable storage medium storing one or more programs, the one or more programs comprising instructions, which when executed by one or more processors of a system, cause the system to:
   receive, using one or more processors, training data comprising training values for a plurality of training input features associated with a receptor gene status in a plurality of training samples;
   train, using the one or more processors, a statistical model based on the training data;
   obtain, using the one or more processors, a set of input features associated with the receptor gene status, wherein obtaining the set of input features comprises omitting input features with a low prevalence and high correlation to the statistical model;
   receive, using one or more processors, sequence read data associated with a sample from an individual;
   determine, using the one or more processors, values for one or more input features associated with the receptor gene status based on the sequence read data, wherein the one or more input features to be evaluated are specified based on a sample type of the sample;
   input, using the one or more processors, the values for the one or more input features into the trained statistical model; and
   predict using the one or more processors, the receptor gene status of the individual based on an output of the trained statistical model.
128. A method for predicting a receptor gene status of a sample from an individual, the method comprising:
   receiving, using one or more processors, sequence read data associated with a sample from an individual;
   obtaining a set of input features associated with the receptor gene status based on an expected prevalence and correlation to the sequence read data and on a sample type associated with the type of sample;
   determining, using the one or more processors, values for one or more input features associated with the receptor gene status based on the sequence read data, wherein the one or more input features to be evaluated are specified based on a sample type of the sample;
   inputting, using the one or more processors, the values for the one or more input features into a statistical model; and
   predicting, using the one or more processors, the receptor gene status of the individual based on an output of the statistical model.
129. The method of clause 128, wherein the receptor gene status corresponds to an estrogen receptor status, a progesterone receptor status, an androgen receptor status, or a human epidermal growth factor receptor 2 status.
130. The method of any of clauses 128 to 129, further comprising applying the statistical model to the values for the one or more input features to obtain an output indicative of the receptor gene status.
131. The method any of clauses 128 to 130, wherein the sequence read data for the individual is derived from a solid sample.
132. The method of clause 131, wherein the sequence read data for the individual is derived from a biopsy sample.
133. The method of clause 131, wherein the set of input features comprises genomic alteration features, complex mutational signatures, chromosomal instability features, clinicopathological features, clinical features, additional features, or a combination thereof.
134. The method any of clauses 131 to 133, wherein the one or more input features are associated with one or more genomic alteration features.
135. The method of clause 134, wherein the one or more genomic alteration features comprise a presence of a predetermined short variant, an absence of a predetermined short variant, a copy number alteration, a rearrangement alteration, an amplification, a deletion, or a combination thereof.
136. The method of clause 135, wherein the predetermined short variant comprises a point mutation, an insertion, or a deletion.
137. The method of any of clauses 131 to 136, wherein the one or more genomic alteration features comprise CCNE1, EGFR, ERBB2, ERBB2 Amplification, ESR1, FGF3, FGFR1, GATA3, MAP2K4, MYC, PIK3CA, PTEN, RB1, TP53, ZNF703, or a combination thereof.
138. The method of any of clauses 131 to 136, wherein the one or more genomic alteration features comprise BRCA1, TP53, MYC, ERBB2, ERBB2 Amplification, ERBB2 Short Variant, PIK3CA, ESR1, TBX3, CDH1, ZNF703, GATA3, FGF3, CCNE1, RB1, FGFR1, PTEN, EGFR, MAP2K4, or a combination thereof.
139. The method of any of clauses 131 to 136, wherein the one or more genomic alteration features comprise AKT1, AKT2, AKT3, APC, AR, ARFRP1, ARID1A, ATM, AURKA, BAP1, BCL2L1, BCL2L2, BRAF, BRCA1, BRCA2, BRD4, CBFB, CCND3, CCNE1, CDH1, CDK12, CDK4, CDK6, CDKN2A, CDKN2B, CHEK2, CREBBP, CTNNA1, DNMT3A, EGFR, EMSY, EP300, ERBB2, ERBB2 Amplification, ERBB2 Short Variant, ERBB3, ESR1, FANCA, FAS, FBXW7, FGF10, FGF12, FGF3, FGF6, FGFR1, FGFR2, FGFR4, GATA3, GNAS, HGF, IGF1R, IKBKE, IRS2, JAK2, KDM5A, KDM6A, KDR, KEL, KIT, KMT2D, KRAS, LYN, MAP2K4, MAP3K1, MCL1, MDM2, MDM4, MSH6, MUTYH, MYC, MYCL, NF1, NFKBIA, NKX2 1, NOTCH1, NOTCH2, NOTCH3,, TRK1, PALB2, PBRM1, PDCD1LG2, PDGFRA, PIK3C2B, PIK3CA, PIK3CB, PIK3R1, PRKCI, PTEN, RAF1, RB1, RET, RICTOR, ROS1, RPTOR, SETD2, SF3B1, SMAD4, SMARCA4, SOX2, SPEN, SRC, STK11, TBX3, TERC, TET2, TP53, TSC1, VEGFA, ZNF217, ZNF703, or a combination thereof.
140. The method of any of clauses 131 to 139, wherein the one or more input features are associated with one or more complex mutational signatures.
141. The method of clause 140, wherein the one or more complex mutational signatures comprise a genome-wide loss of heterozygosity (gLOH) quantification, trinucleotide signatures, insertion signatures, deletion signatures, copy number signatures, or a combination thereof.
142. The method of any of clauses 131 to 141, wherein the one or more input features are associated with one or more chromosomal instability features.
143. The method of clause 142, wherein the one or more chromosomal instability features is indicative of aneuploidy.
144. The method of clause 142, wherein the one or more chromosomal instability features is indicative of chromosome gain, chromosome not gain, chromosome loss, chromosome not loss, or a combination thereof.
145. The method of clause 142, wherein the one or more chromosomal instability features comprises a total aneuploidy count.
146. The method of clause 142, wherein the one or more chromosomal instability features comprise chromosome 1q loss status, chromosome 1p loss status, chromosome 3p loss status, chromosome 4p loss status, chromosome 5q loss status, chromosome 9p loss status, chromosome 10q loss status, chromosome 11q loss status, chromosome 12q loss status, chromosome 14q loss status, chromosome 15q loss status, chromosome 16p loss status, chromosome 16q loss status, chromosome 17q loss status, chromosome 19p loss status, chromosome 21q loss status, chromosome 22q loss status, chromosome 1q gain status, chromosome 2p gain status, chromosome 5p gain status, chromosome 5q gain status, chromosome 6q gain status, chromosome 7q gain status, chromosome 9p gain status, chromosome 10p gain status, chromosome 12q gain status, chromosome 16p gain status, chromosome 18p gain status, chromosome 20q gain status chromosome 21p gain status, chromosome 21q gain status.
147. The method of clause 142, wherein the one or more chromosomal instability features comprise chromosome 1p gain status, chromosome 1q gain status, chromosome2p gain status, chromosome 2q gain status, chromosome 3p gain status, chromosome 3q gain status, chromosome 4p gain status, chromosome 4q gain status, chromosome 5p gain status, chromosome 5q gain status, chromosome 6p gain status, chromosome 6q gain status, chromosome 7p gain status, chromosome 7q gain status, chromosome 8p gain status, chromosome 8q gain status, chromosome 9p gain status, chromosome 9q gain status, chromosome 10p gain status, chromosome 10q gain status, chromosome 11p gain status, chromosome 11q gain status, chromosome 12p gain status, chromosome 12q gain status, chromosome 13q gain status, chromosome 14q gain status, chromosome 15q gain status, chromosome 16p gain status, chromosome 16q gain status, chromosome 17p gain status, chromosome 17q gain status, chromosome 18p gain status, chromosome 18q gain status, chromosome 19p gain status, chromosome 19q gain status, chromosome 20p gain status, chromosome 20q gain status, chromosome 21p gain status, chromosome 21q gain status, chromosome 22q gain status, chromosome 1p loss status, chromosome 2p loss status, chromosome 2q loss status, chromosome 3p loss status, chromosome 3q loss status, chromosome 4p loss status, chromosome 4q loss status, chromosome 5p loss status, chromosome 5q loss status, chromosome 6p loss status, chromosome 6q loss status, chromosome 7p loss status, chromosome 7q loss status, chromosome 8p loss status, chromosome 8q loss status, chromosome 9p loss status, chromosome 9q loss status, chromosome 10p loss status, chromosome 10q loss status, chromosome 11p loss status, chromosome 11q loss status, chromosome 12p loss status, chromosome 12q loss status, chromosome 13q loss status, chromosome 14q loss status, chromosome 15q loss status, chromosome 16p loss status, chromosome 16q loss status, chromosome 17p loss status, chromosome 17q loss status, chromosome 18p loss status, chromosome 18q loss status, chromosome 19p loss status, chromosome 19q loss status, chromosome 20p loss status, chromosome 20q loss status, chromosome 21p loss status, chromosome 21q loss status, chromosome 22q loss status.
148. The method of any of clauses 131 to 147, wherein the one or more input features are associated with one or more clinicopathological features.
149. The method of clause 148, wherein the one or more clinicopathological features comprise an age of the individual, a sex of the individual, a disease diagnosis of the individual, a tumor status of the individual, a tumor type, an anatomical sub-classification of a tumor, or a combination thereof.
150. The method of any of clauses 131 to 149, wherein the one or more input features are associated with one or more clinical features.
151. The method of clause 150, wherein the one or more clinical features comprise an ancestry of the individual, a family history of the individual, a stage of the disease, one or more laboratory-based test results, one or more lifestyle habits, a diabetes status, an obesity status, an indication of body vitamin levels, or a combination thereof.
152. The method of any of clauses 131 to 151, wherein the one or more input features are associated with a tumor mutational burden.
153. The method of any of clauses 131 to 152, wherein the one or more input features are associated with a germline status.
154. The method of any of clauses 131 to 153, wherein the one or more input features are associated with a methylation signature, a mRNA expression level, a miRNA expression level, proteomics, COSMIC mutation signatures, immunohistochemical markers, genetic predispositions, cell adhesion biomarkers, saliva based biomarkers, enzyme based biomarkers, a diagnostic image, a homologous repair deficiency (HRD) signature, or a combination thereof.
155. The method of any of clauses 131 to 154, wherein sequence read data derived from a liquid sample is associated with a first set of one or more input features and sequence read data derived from a solid sample is associated with a second set of one or more input features.
156. The method any of clauses 131 to 155, wherein the sequence read data for the individual is derived from a liquid sample.
157. The method of clause 156, wherein the sequence read data for the individual is derived from a liquid biopsy sample.
158. The method of any of clauses 156 to 157, wherein the set of input features comprises genomic alteration features, clinicopathological features, clinical features, tumor fraction features, fragmentomic features, additional features, or a combination thereof.
159. The method any of clauses 156 to 158, wherein the one or more input features are associated with one or more genomic alteration features.
160. The method of clause 159, wherein the one or more genomic alteration features comprise a presence of a predetermined short variant, an absence of a predetermined short variant, a rearrangement alteration, an insertion, a deletion, or a combination thereof.
161. The method of any of clauses 159 to 160, wherein the one or more genomic alteration features comprise TP53, ESR1, PIK3CA, or a combination thereof.
162. The method of clause 159 to 161, wherein the one or more genomic alteration features comprise CDH1, BRCA1, AKT1, BRCA2, CDKN2A, ERBB2, KRAS, NF1, PTEN, or a combination thereof.
163. The method of any of clauses 159 to 162, wherein the one or more input features are associated with one or more clinicopathological features.
164. The method of clause 163, wherein the one or more clinicopathological features comprise an age of the individual, a sex of the individual, a disease diagnosis of the individual, a tumor type, or a combination thereof.
165. The method of any of clauses 159 to 164, wherein the one or more input features are associated with one or more clinical features.
166. The method of clause 165, wherein the one or more clinical features comprise an ancestry of the individual, a stage of the disease, one or more laboratory-based test results, one or more lifestyle habits, a diabetes status, an obesity status, an indication of body vitamin levels, or a combination thereof.
167. The method of any of clauses 159 to 166, wherein the one or more input features are associated with a tumor mutational burden.
168. The method of any of clauses 159 to 167, wherein the one or more input features are associated with trinucleotide signatures, insertion signatures, deletion signatures, copy number signatures, or a combination thereof.
169. The method of any of clauses 159 to 168, wherein the one or more input features are associated with the HRD signature.
170. The method of any of clauses 159 to 169, wherein the one or more input features are associated with one or more fragmentomic features, the one or more fragmentomic features associated with a plurality of reads obtained from the sequence read data.
171. The method of clause 170, wherein the one or more fragmentomic features comprise lengths of the plurality reads, regions associated with the plurality of reads, or a combination thereof.
172. The method of clause 170 to 171, wherein the one or more fragmentomic features comprise an amount of a fragment having a specified length, a mean fragment length of the plurality of reads, a median fragment length of the plurality of reads, an interquartile range of fragment lengths of the plurality of reads, a distribution of fragment lengths of the plurality of reads, one or more peaks of fragment lengths for the plurality of reads, a fragment length for the plurality of reads, a start position of a fragment for the plurality of reads, and an end position of a fragment for the plurality of reads.
173. The method of any of clauses 156 to 172, wherein the one or more input features are associated with an estimated tumor fraction.
174. The method of any of clauses 156 to 173, wherein the one or more input features are associated with a methylation signature, a mRNA expression level, a miRNA expression level, proteomics, COSMIC mutation signatures, immunohistochemical markers, genetic predispositions, cell adhesion biomarkers, saliva based biomarkers, enzyme based biomarkers, a diagnostic image, or a combination thereof.
175. The method of any of clauses 128 to 174, wherein the output of the statistical model is indicative of a receptor status.
176. The method of clause 175, wherein the output of the statistical model comprises a first score indicative of a probability of a positive receptor gene status.
177. The method of any of clauses 175 to 176, wherein the output of the statistical model comprises a second score indicative of a probability of a negative receptor gene status.
178. The method of any of clauses 128 to 177, wherein the statistical model is a trained machine learning model and trained by:
   receiving, using the one or more processors, training data including a plurality of training values for a plurality of input features corresponding to a plurality of training samples; and
   training, using the one or more processors, the machine learning model based on the training data.
179. The method of the clause 178, wherein the training data further comprises receptor gene statuses corresponding to each of the plurality of training samples.
180. The method of any of clauses 178 to 179, further comprising:
   determining, using the one or more processors, weights associated with the training values for the plurality of training expression input features based on the training;
   filtering, using the one or more processors, the plurality of training input features based on the weights; and
   determining, using the one or more processors, the set of input features based on the filtered plurality of training input features, wherein filtering the plurality of training input features comprises removing low prevalence training values, highly correlated training values, or a combination thereof.
181. The method of clause 180, further comprising weighting, using the one or more processors, the values for the one or more input features based on the weights, wherein predicting the receptor gene status is based on the weighted values for the one or more input features.
182. The method of any of clauses 128 to 181, wherein the receptor gene status comprises a hormone receptor status.
183. The method of any of clauses 128 to 182, wherein the statistical model is a trained machine learning model, a part of a machine learning process, or a combination thereof.
184. The method of any of clauses 128 to 183, wherein the statistical model includes an artificial intelligence learning model.
185. The method of any of clauses 128 to 184, wherein the statistical model comprises a random forest model.
186. The method of any of clauses 128 to 185, wherein the statistical model comprises at least one of an extreme gradient boosting model, a logistic regression model, an elastic net model, a ridge regression model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.
187. The method of any of clauses 128 to 186, further comprising:
   inputting, using the one or more processors, a diagnostic image taken of the individual into a second statistical model; and
   determining, using the one or more processors, a third score indicative of a tumor classification based on the diagnostic image,
   wherein predicting the receptor gene status is further based on the third score.
188. The method of clause 187, wherein the second statistical model is at least one of a deep learning model, a convolutional neural network model, a recurrent neural network model, and an auto-encoder model.
189. The method of any of clauses 187 to 188, further comprising training the second statistical model, wherein the second statistical model is trained by:
   receiving, using the one or more processors, training data including a plurality of training diagnostic images; and
   training, using the one or more processors, the second statistical model based on the training data.
190. The method of any of clauses 187 to 189, wherein the diagnostic image comprises at least one of a histopathological image, a radiological image, a magnetic resonance image, an ultrasound image, a X-ray image, a bone scan, a CT scan, a PET scan, or a combination thereof.
191. The method any of clauses 128 to 190, further comprising assigning, using the one or more processors, a therapy for the individual based on the predicted receptor gene status.
192. The method any of clauses 128 to 191, further comprising determining, using the one or more processors, a treatment decision for the individual based on the predicted receptor gene status.
193. The method any of clauses 128 to 192, further comprising administering, using the one or more processors, a treatment to the individual based on the predicted receptor gene status.
194. The method any of clauses 128 to 193, further comprising monitoring, using the one or more processors, a prognosis of the individual based on the predicted receptor gene status.
195. The method any of clauses 194, further comprising predicting, using the one or more processors, one or more clinical outcomes based on the predicted receptor gene status.
196. A method for diagnosing a disease, the method comprising:
   diagnosing that a subject has the disease based on a determination of a receptor gene status for a sample from the subject, wherein the receptor gene status is determined according to the method of any one of clauses 128 to 195.
197. A method of selecting an anti-cancer therapy, the method comprising:
   responsive to determining a receptor gene status for a sample from a subject, selecting an anti-cancer therapy for the subject, wherein the receptor gene status is determined according to the method of any one of clauses 128 to 195.
198. A method of treating a cancer in a subject, comprising:
   responsive to determining a receptor gene status for a sample from the subject, administering an effective amount of an anti-cancer therapy to the subject, wherein the receptor gene status is determined according to the method of any one of clauses 128 to 195.
199. A method for monitoring cancer progression or recurrence in a subject, the method comprising:
   determining a first receptor gene status in a first sample obtained from the subject at a first time point according to the method of any one of clauses 128 to 195;
   determining a second receptor gene status in a second sample obtained from the subject at a second time point; and comparing the first receptor gene status to the second receptor gene status, thereby monitoring the cancer progression or recurrence.
200. The method of clause 199, wherein the second receptor gene status for the second sample is determined according to the method of any one of clauses 128 to 195.
201. The method of any of clauses 199 to 200, further comprising selecting an anti-cancer therapy for the subject in response to the cancer progression.
202. The method of clauses 199 to 200, further comprising administering an anti-cancer therapy to the subject in response to the cancer progression.
203. The method of clause 199 to 200, further comprising adjusting an anti-cancer therapy for the subject in response to the cancer progression.
204. The method of any one of clauses 201-203, further comprising adjusting a dosage of the anti-cancer therapy or selecting a different anti-cancer therapy in response to the cancer progression.
205. The method of clause 204, further comprising administering the adjusted anti-cancer therapy to the subject.
206. The method of any one of clauses 199 to 205, wherein the first time point is before the subject has been administered an anti-cancer therapy, and wherein the second time point is after the subject has been administered the anti-cancer therapy.
207. The method of any one of clauses 199 to 206, wherein the subject has a cancer, is at risk of having a cancer, is being routine tested for cancer, or is suspected of having a cancer.
208. The method of any one of clauses 199 to 207, wherein the cancer is a solid tumor.
209. The method of any one of clauses 199 to 207, wherein the cancer is a breast cancer.
210. The method of any one of clauses 199 to 209, wherein the anti-cancer therapy comprises chemotherapy, radiation therapy, immunotherapy, a targeted therapy, or surgery.
211. The method of any one of clauses 128 to 195, further comprising determining, identifying, or applying the receptor gene status for the sample as a diagnostic value associated with the sample.
212. The method of any one of clauses 128 to 195, further comprising generating a genomic profile for the subject based on the determination of the receptor gene status.
213. The method of clause 212, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
214. The method of any of clause 212 or clause 213, wherein the genomic profile for the subject further comprises results from a nucleic acid sequencing-based test.
215. The method of any of clauses 212 to 214, further comprising selecting an anti-cancer therapy, administering an anti-cancer therapy, or applying an anti-cancer therapy to the subject based on the generated genomic profile.
216. The method of any one of clauses 128 to 195, wherein the determination of receptor gene status for the sample is used in making suggested treatment decisions for the subject.
217. The method of any one of clauses 128 to 195, wherein the determination of the receptor gene status for the sample is used in applying or administering a treatment to the subject.
218. A system comprising:
   one or more processors; and
   a memory communicatively coupled to the one or more processors and configured to store instructions that, when executed by the one or more processors, cause the system to:
      receive, using one or more processors, sequence read data associated with a sample from an individual;
      obtain a set of input features associated with the receptor gene status based on an expected prevalence and correlation to the sequence read data and on a sample type associated with the type of sample;
      determine, using the one or more processors, values for one or more input features associated with the receptor gene status based on the sequence read data, wherein the one or more input features to be evaluated are specified based on a sample type of the sample;
      input, using the one or more processors, the values for the one or more input features into a statistical model; and
      predict, using the one or more processors, the receptor gene status of the individual based on an output of the statistical model.
219. A non-transitory computer-readable storage medium storing one or more programs, the one or more programs comprising instructions, which when executed by one or more processors of a system, cause the system to:
   receive, using one or more processors, sequence read data associated with a sample from an individual;
   obtain a set of input features associated with the receptor gene status based on an expected prevalence and correlation to the sequence read data and on a sample type associated with the type of sample;
   determine, using the one or more processors, values for one or more input features associated with the receptor gene status based on the sequence read data, wherein the one or more input features to be evaluated are specified based on a sample type of the sample;
   input, using the one or more processors, the values for the one or more input features into a statistical model; and
   predict, using the one or more processors, the receptor gene status of the individual based on an output of the statistical model.
220. A method comprising:
   receiving, using one or more processors, sequence read data associated with a sample from an individual;
   obtaining a set of expression input features associated with a receptor gene status based on an expected prevalence and correlation to the sequence read data and on a sample type associated with the type of sample;
   determining, using the one or more processors, values for one or more expression input features associated with the receptor gene status based on the sequence read data, wherein the expression input features to be evaluated are specified based on a sample type of the sample;
   inputting, using the one or more processors, the values for the one or more expression input features into the statistical model; and
   predicting, using the one or more processors, the receptor gene status of the individual based on an output of the statistical model associated with the one or more expression input features.
221. The method of clause 220, wherein the receptor gene status corresponds to an estrogen receptor status, a progesterone receptor status, an androgen receptor status, or a human epidermal growth factor receptor 2 status.
222. The method of any of clauses 220 to 221, further comprising applying the machine learning model to the values for the one or more expression input features to obtain an output indicative of the receptor gene status.
223. The method any of clauses 220 to 222, wherein the sequence read data for the individual is derived from a solid sample.
224. The method of clause 223, wherein the sequence read data for the individual is derived from a biopsy sample.
225. The method of clause 224, wherein the set of input features comprises genomic alteration features, complex mutational signatures, chromosomal instability features, clinicopathological features, clinical features, additional features, or a combination thereof.
226. The method any of clauses 223 to 225, wherein the one or more expression input features are associated with one or more genomic alteration features.
227. The method of clause 226, wherein the one or more genomic alteration features comprise a presence of a predetermined short variant, an absence of a predetermined short variant, a copy number alteration, a rearrangement alteration, an amplification, a deletion, or a combination thereof.
228. The method of clause 227, wherein the predetermined short variant comprises a point mutation, an insertion, or a deletion.
229. The method of any of clauses 226 to 228, wherein the one or more genomic alteration features comprise CCNE1, EGFR, ERBB2, ERBB2 Amplification, ESR1, FGF3, FGFR1, GATA3, MAP2K4, MYC, PIK3CA, PTEN, RB1, TP53, ZNF703, or a combination thereof.
230. The method of any of clauses 226 to 228, wherein the one or more genomic alteration features comprise BRCA1, TP53, MYC, ERBB2, ERBB2 Amplification, ERBB2 Short Variant, PIK3CA, ESR1, TBX3, CDH1, ZNF703, GATA3, FGF3, CCNE1, RB1, FGFR1, PTEN, EGFR, MAP2K4, or a combination thereof.
231. The method of any of clauses 226 to 228, wherein the one or more genomic alteration features comprise AKT1, AKT2, AKT3, APC, AR, ARFRP1, ARID1A, ATM, AURKA, BAP1, BCL2L1, BCL2L2, BRAF, BRCA1, BRCA2, BRD4, CBFB, CCND3, CCNE1, CDH1, CDK12, CDK4, CDK6, CDKN2A, CDKN2B, CHEK2, CREBBP, CTNNA1, DNMT3A, EGFR, EMSY, EP300, ERBB2, ERBB2 Amplification, ERBB2 Short Variant, ERBB3, ESR1, FANCA, FAS, FBXW7, FGF10, FGF12, FGF3, FGF6, FGFR1, FGFR2, FGFR4, GATA3, GNAS, HGF, IGF1R, IKBKE, IRS2, JAK2, KDM5A, KDM6A, KDR, KEL, KIT, KMT2D, KRAS, LYN, MAP2K4, MAP3K1, MCL1, MDM2, MDM4, MSH6, MUTYH, MYC, MYCL, NF1, NFKBIA, NKX2 1, NOTCH1, NOTCH2, NOTCH3,, TRK1, PALB2, PBRM1, PDCD1LG2, PDGFRA, PIK3C2B, PIK3CA, PIK3CB, PIK3R1, PRKCI, PTEN, RAF1, RB1, RET, RICTOR, ROS1, RPTOR, SETD2, SF3B1, SMAD4, SMARCA4, SOX2, SPEN, SRC, STK11, TBX3, TERC, TET2, TP53, TSC1, VEGFA, ZNF217, ZNF703, or a combination thereof.
232. The method of any of clauses 223 to 231, wherein the one or more expression input features are associated with one or more complex mutational signatures.
233. The method of clause 232, wherein the one or more complex mutational signatures comprise a genome-wide loss of heterozygosity (gLOH) quantification, trinucleotide signatures, insertion signatures, deletion signatures, copy number signatures, or a combination thereof.
234. The method of any of clauses 223 to 233, wherein the one or more expression input features are associated with one or more chromosomal instability features.
235. The method of clause 234, wherein the one or more chromosomal instability features is indicative of aneuploidy.
236. The method of clause 235, wherein the one or more chromosomal instability features is indicative of chromosome gain, chromosome not gain, chromosome loss, chromosome not loss, or a combination thereof.
237. The method of clause 235, wherein the one or more chromosomal instability features comprises a total aneuploidy count.
238. The method of clause 235, wherein the one or more chromosomal instability features comprise chromosome 1q loss status, chromosome 1p loss status, chromosome 3p loss status, chromosome 4p loss status, chromosome 5q loss status, chromosome 9p loss status, chromosome 10q loss status, chromosome 11q loss status, chromosome 12q loss status, chromosome 14q loss status, chromosome 15q loss status, chromosome 16p loss status, chromosome 16q loss status, chromosome 17q loss status, chromosome 19p loss status, chromosome 21q loss status, chromosome 22q loss status, chromosome 1q gain status, chromosome 2p gain status, chromosome 5p gain status, chromosome 5q gain status, chromosome 6q gain status, chromosome 7q gain status, chromosome 9p gain status, chromosome 10p gain status, chromosome 12q gain status, chromosome 16p gain status, chromosome 18p gain status, chromosome 20q gain status chromosome 21p gain status, chromosome 21q gain status.
239. The method of clause 235, wherein the one or more chromosomal instability features comprise chromosome 1p gain status, chromosome 1q gain status, chromosome2p gain status, chromosome 2q gain status, chromosome 3p gain status, chromosome 3q gain status, chromosome 4p gain status, chromosome 4q gain status, chromosome 5p gain status, chromosome 5q gain status, chromosome 6p gain status, chromosome 6q gain status, chromosome 7p gain status, chromosome 7q gain status, chromosome 8p gain status, chromosome 8q gain status, chromosome 9p gain status, chromosome 9q gain status, chromosome 10p gain status, chromosome 10q gain status, chromosome 11p gain status, chromosome 11q gain status, chromosome 12p gain status, chromosome 12q gain status, chromosome 13q gain status, chromosome 14q gain status, chromosome 15q gain status, chromosome 16p gain status, chromosome 16q gain status, chromosome 17p gain status, chromosome 17q gain status, chromosome 18p gain status, chromosome 18q gain status, chromosome 19p gain status, chromosome 19q gain status, chromosome 20p gain status, chromosome 20q gain status, chromosome 21p gain status, chromosome 21q gain status, chromosome 22q gain status, chromosome 1p loss status, chromosome 2p loss status, chromosome 2q loss status, chromosome 3p loss status, chromosome 3q loss status, chromosome 4p loss status, chromosome 4q loss status, chromosome 5p loss status, chromosome 5q loss status, chromosome 6p loss status, chromosome 6q loss status, chromosome 7p loss status, chromosome 7q loss status, chromosome 8p loss status, chromosome 8q loss status, chromosome 9p loss status, chromosome 9q loss status, chromosome 10p loss status, chromosome 10q loss status, chromosome 11p loss status, chromosome 11q loss status, chromosome 12p loss status, chromosome 12q loss status, chromosome 13q loss status, chromosome 14q loss status, chromosome 15q loss status, chromosome 16p loss status, chromosome 16q loss status, chromosome 17p loss status, chromosome 17q loss status, chromosome 18p loss status, chromosome 18q loss status, chromosome 19p loss status, chromosome 19q loss status, chromosome 20p loss status, chromosome 20q loss status, chromosome 21p loss status, chromosome 21q loss status, chromosome 22q loss status.
240. The method of any of clauses 223 to 239, wherein the one or more expression input features are associated with one or more clinicopathological features.
241. The method of clause 240, wherein the one or more clinicopathological features comprise an age of the individual, a sex of the individual, a disease diagnosis of the individual, a tumor status of the individual, a tumor type, an anatomical sub-classification of a tumor, or a combination thereof.
242. The method of any of clauses 223 to 241, wherein the one or more expression input features are associated with one or more clinical features.
243. The method of clause 242, wherein the one or more clinical features comprise an ancestry of the individual, a family history of the individual, a stage of the disease, one or more laboratory-based test results, one or more lifestyle habits, a diabetes status, an obesity status, an indication of body vitamin levels, or a combination thereof.
244. The method of any of clauses 223 to 243, wherein the one or more expression input features are associated with a tumor mutational burden.
245. The method of any of clauses 223 to 244, wherein the one or more expression input features are associated with a germline status.
246. The method of any of clauses 223 to 245, wherein the one or more expression input features are associated with a methylation signature, a mRNA expression level, a miRNA expression level, proteomics, COSMIC mutation signatures, immunohistochemical markers, genetic predispositions, cell adhesion biomarkers, saliva based biomarkers, enzyme based biomarkers, a diagnostic image, a homologous repair deficiency (HRD) signature, or a combination thereof.
247. The method of any of clauses 223 to 246, wherein sequence read data derived from a liquid sample is associated with a first set of one or more input features and sequence read data derived from a solid sample is associated with a second set of one or more input features.
248. The method any of clauses 223 to 247, wherein the sequence read data for the individual is derived from a liquid sample.
249. The method of clause 248, wherein the sequence read data for the individual is derived from a liquid biopsy sample.
250. The method of any of clauses 248 to 249, wherein the set of input features comprises genomic alteration features, clinicopathological features, clinical features, tumor fraction features, fragmentomic features, additional features, or a combination thereof.
251. The method any of clauses 248 to 250, wherein the one or more expression input features are associated with one or more genomic alteration features.
252. The method of clause 251, wherein the one or more genomic alteration features comprise a presence of a predetermined short variant, an absence of a predetermined short variant, a rearrangement alteration, an insertion, a deletion, or a combination thereof.
253. The method of any of clauses 251 to 252, wherein the one or more genomic alteration features comprise TP53, ESR1, PIK3CA, or a combination thereof.
254. The method of clause 251 to 252, wherein the one or more genomic alteration features comprise CDH1, BRCA1, AKT1, BRCA2, CDKN2A, ERBB2, KRAS, NF1, PTEN, or a combination thereof.
255. The method of any of clauses 248 to 254, wherein the one or more expression input features are associated with one or more clinicopathological features.
256. The method of clause 255, wherein the one or more clinicopathological features comprise an age of the individual, a sex of the individual, a disease diagnosis of the individual, a tumor type, or a combination thereof.
257. The method of any of clauses 248 to 256, wherein the one or more expression input features are associated with one or more clinical features.
258. The method of clause 257, wherein the one or more clinical features comprise an ancestry of the individual, a stage of the disease, one or more laboratory-based test results, one or more lifestyle habits, a diabetes status, an obesity status, an indication of body vitamin levels, or a combination thereof.
259. The method of any of clauses 248 to 258, wherein the one or more expression input features are associated with a tumor mutational burden.
260. The method of any of clauses 248 to 259, wherein the one or more expression input features are associated with trinucleotide signatures, insertion signatures, deletion signatures, copy number signatures, or a combination thereof.
261. The method of any of clauses 248 to 260, wherein the one or more expression input features are associated with the HRD signature.
262. The method of any of clauses 248 to 261, wherein the one or more expression input features are associated with one or more fragmentomic features, the one or more fragmentomic features associated with a plurality of reads obtained from the sequence read data.
263. The method of clause 262, wherein the one or more fragmentomic features comprise lengths of the plurality reads, regions associated with the plurality of reads, or a combination thereof.
264. The method of clause 242 to 263, wherein the one or more fragmentomic features comprise an amount of a fragment having a specified length, a mean fragment length of the plurality of reads, a median fragment length of the plurality of reads, an interquartile range of fragment lengths of the plurality of reads, a distribution of fragment lengths of the plurality of reads, one or more peaks of fragment lengths for the plurality of reads, a fragment length for the plurality of reads, a start position of a fragment for the plurality of reads, and an end position of a fragment for the plurality of reads.
265. The method of any of clauses 248 to 264, wherein the one or more expression input features are associated with an estimated tumor fraction.
266. The method of any of clauses 248 to 265, wherein the one or more expression input features are associated with a methylation signature, a mRNA expression level, a miRNA expression level, proteomics, COSMIC mutation signatures, immunohistochemical markers, genetic predispositions, cell adhesion biomarkers, saliva based biomarkers, enzyme based biomarkers, a diagnostic image, or a combination thereof.
267. The method of any of clauses 220 to 266, wherein the output of the statistical model is indicative of a receptor status.
268. The method of clause 267, wherein the output of the statistical model comprises a first score indicative of a probability of a positive receptor gene status.
269. The method of any of clauses 267 to 268, wherein the output of the statistical model comprises a second score indicative of a probability of a negative receptor gene status.
270. The method of any of clauses 220 to 269, wherein the statistical model is a trained machine learning model and trained by:
   receiving, using the one or more processors, training data including a plurality of training values for one or more training expression input features corresponding to a plurality of training samples; and
   training, using the one or more processors, the machine learning model based on the training data.
271. The method of the clause 270, wherein the training data further comprises receptor gene statuses corresponding to each of the plurality of training samples.
272. The method of any of clauses 270 to 271, further comprising:
   determining, using the one or more processors, weights associated with the training values for the plurality of training expression input features based on the training; and
   filtering, using the one or more processors, the plurality of training input features based on the weights;
   determining, using the one or more processors, the set of input features based on the filtered plurality of training input features, wherein filtering the plurality of training input features comprises removing low prevalence training values, highly correlated training values, or a combination thereof.
273. The method of clause 272, further comprising weighting, using the one or more processors, the values for the one or more expression input features based on the weights, wherein predicting the receptor gene status is based on the weighted values for the one or more expression input features.
274. The method of any of clauses 220 to 273, wherein the receptor gene status comprises a hormone receptor status.
275. The method of any of clauses 220 to 274, wherein the statistical model is a trained machine learning model, a part of a machine learning process, or a combination thereof.
276. The method of any of clauses 220 to 275, wherein the statistical model includes an artificial intelligence learning model.
277. The method of any of clauses 220 to 276, wherein the statistical model comprises a random forest model.
278. The method of any of clauses 220 to 277, wherein the statistical model comprises at least one of an extreme gradient boosting model, a logistic regression model, an elastic net model, a ridge regression model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.
279. The method of any of clauses 220 to 278, further comprising:
   inputting, using the one or more processors, a diagnostic image taken of the individual into a second statistical model; and
   determining, using the one or more processors, a third score indicative of a tumor classification based on the diagnostic image,
   wherein predicting the receptor gene status is further based on the third score.
280. The method of clause 279, wherein the second statistical model is at least one of a deep learning model, a convolutional neural network model, a recurrent neural network model, and an auto-encoder model.
281. The method of any of clauses 279 to 280, further comprising training the second statistical model, wherein the second statistical model is trained by:
   receiving, using the one or more processors, training data including a plurality of training diagnostic images; and
   training, using the one or more processors, the second statistical model based on the training data.
282. The method of any of clauses 279 to 281, wherein the diagnostic image comprises at least one of a histopathological image, a radiological image, a magnetic resonance image, an ultrasound image, a X-ray image, a bone scan, a CT scan, a PET scan, or a combination thereof.
283. The method any of clauses 220 to 282, further comprising assigning, using the one or more processors, a therapy for the individual based on the predicted receptor gene status.
284. The method any of clauses 220 to 283, further comprising determining, using the one or more processors, a treatment decision for the individual based on the predicted receptor gene status.
285. The method any of clauses 220 to 284, further comprising administering, using the one or more processors, a treatment to the individual based on the predicted receptor gene status.
286. The method any of clauses 220 to 285, further comprising monitoring, using the one or more processors, a prognosis of the individual based on the predicted receptor gene status.
287. The method any of clauses 286, further comprising predicting, using the one or more processors, one or more clinical outcomes based on the predicted receptor gene status.
288. A method for diagnosing a disease, the method comprising:
   diagnosing that a subject has the disease based on a determination of a receptor gene status for a sample from the subject, wherein the receptor gene status is determined according to the method of any one of clauses 220 to 287.
289. A method of selecting an anti-cancer therapy, the method comprising:
   responsive to determining a receptor gene status for a sample from a subject, selecting an anti-cancer therapy for the subject, wherein the receptor gene status is determined according to the method of any one of clauses 220 to 287.
290. A method of treating a cancer in a subject, comprising:
   responsive to determining a receptor gene status for a sample from the subject, administering an effective amount of an anti-cancer therapy to the subject, wherein the receptor gene status is determined according to the method of any one of clauses 220 to 287.
291. A method for monitoring cancer progression or recurrence in a subject, the method comprising:
   determining a first receptor gene status in a first sample obtained from the subject at a first time point according to the method of any one of clauses 220 to 287;
   determining a second receptor gene status in a second sample obtained from the subject at a second time point; and comparing the first receptor gene status to the second receptor gene status, thereby monitoring the cancer progression or recurrence.
292. The method of clause 291, wherein the second receptor gene status for the second sample is determined according to the method of any one of clauses 220 to 287.
293. The method of any of clauses 291 to 292, further comprising selecting an anti-cancer therapy for the subject in response to the cancer progression.
294. The method of clauses 291 to 292, further comprising administering an anti-cancer therapy to the subject in response to the cancer progression.
295. The method of clause 291 to 292, further comprising adjusting an anti-cancer therapy for the subject in response to the cancer progression.
296. The method of any one of clauses 293 to 295, further comprising adjusting a dosage of the anti-cancer therapy or selecting a different anti-cancer therapy in response to the cancer progression.
297. The method of clause 296, further comprising administering the adjusted anti-cancer therapy to the subject.
298. The method of any one of clauses 291 to 297, wherein the first time point is before the subject has been administered an anti-cancer therapy, and wherein the second time point is after the subject has been administered the anti-cancer therapy.
299. The method of any one of clauses 291 to 298, wherein the subject has a cancer, is at risk of having a cancer, is being routine tested for cancer, or is suspected of having a cancer.
300. The method of any one of clauses 281 to 289, wherein the cancer is a solid tumor.
301. The method of any one of clauses 291 to 300, wherein the cancer is a breast cancer.
302. The method of any one of clauses 291 to 301, wherein the anti-cancer therapy comprises chemotherapy, radiation therapy, immunotherapy, a targeted therapy, or surgery.
303. The method of any one of clauses 291 to 302, further comprising determining, identifying, or applying the receptor gene status for the sample as a diagnostic value associated with the sample.
304. The method of any one of clauses 291 to 303, further comprising generating a genomic profile for the subject based on the determination of the receptor gene status.
305. The method of clause 304, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
306. The method of any of clauses 304 to 305, wherein the genomic profile for the subject further comprises results from a nucleic acid sequencing-based test.
307. The method of any of clauses 304 to 306, further comprising selecting an anti-cancer therapy, administering an anti-cancer therapy, or applying an anti-cancer therapy to the subject based on the generated genomic profile.
308. The method of any one of clauses 220 to 287, wherein the determination of receptor gene status for the sample is used in making suggested treatment decisions for the subject.
309. The method of any one of clauses 220 to 287, wherein the determination of the receptor gene status for the sample is used in applying or administering a treatment to the subject.
310. A system comprising:
   one or more processors; and
   a memory communicatively coupled to the one or more processors and configured to store instructions that, when executed by the one or more processors, cause the system to:
      receive, using one or more processors, sequence read data associated with a sample from an individual;
      obtain a set of expression input features associated with a receptor gene status based on an expected prevalence and correlation to the sequence read data and on a sample type associated with the type of sample;
      determine, using the one or more processors, values for one or more expression input features associated with the receptor gene status based on the sequence read data, wherein the expression input features to be evaluated are specified based on a sample type of the sample;
      input, using the one or more processors, the values for the one or more expression input features into the statistical model; and
      predict, using the one or more processors, the receptor gene status of the individual based on an output of the statistical model associated with the one or more expression input features.
311. A non-transitory computer-readable storage medium storing one or more programs, the one or more programs comprising instructions, which when executed by one or more processors of a system, cause the system to:
   receive, using one or more processors, sequence read data associated with a sample from an individual;
   obtain a set of expression input features associated with a receptor gene status based on an expected prevalence and correlation to the sequence read data and on a sample type associated with the type of sample;
   determine, using the one or more processors, values for one or more expression input features associated with the receptor gene status based on the sequence read data, wherein the expression input features to be evaluated are specified based on a sample type of the sample;
   input, using the one or more processors, the values for the one or more expression input features into the statistical model; and
   predict, using the one or more processors, the receptor gene status of the individual based on an output of the statistical model associated with the one or more expression input features.
312. A method comprising:
   receiving, using one or more processors, training data comprising values for a plurality of training input features associated with a receptor gene status in a plurality of training samples;
   training, using the one or more processors, a statistical model based on the training data, wherein the trained statistical model is configured to predict a receptor gene status of an individual sample;
   determining, using the one or more processors, weights associated with the training values for the plurality of training input features based on the training;
   filtering, using the one or more processors, the one or more training input features based on the weights;
   determining a set of input features associated with the receptor gene status based on the filtered training input features and a sample type of a sample from an individual, wherein filtering the one or more training input features comprises removing training input features associated with low prevalence training values and highly correlated training values, or a combination thereof; and
   obtaining a trained statistical model configured to receive a set of input feature based on a sample from an individual to output a prediction of a receptor gene status of the sample.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of reads that represent the captured nucleic acid molecules;
receiving, at one or more processors, sequence read data for the plurality of reads;
receiving, using one or more processors, training data comprising training values for a plurality of training input features associated with a receptor gene status in a plurality of training samples;
training, using the one or more processors, a statistical model based on the training data;
obtaining, using the one or more processors, a set of input features associated with the receptor gene status, wherein obtaining the set of input features comprises omitting input features with a low prevalence and high correlation to the statistical model;
determining, using the one or more processors, values for one or more input features associated with the receptor gene status based on the sequence read data, wherein the one or more input features to be evaluated are specified based on a sample type of the sample;
inputting, using the one or more processors, the values for the one or more input features into the trained statistical model; and
predicting, using the one or more processors, the receptor gene status of the subject based on an output of the trained statistical model.

2. The method of claim 1, wherein the receptor gene status corresponds to an estrogen receptor status, a progesterone receptor status, an androgen receptor status, or a human epidermal growth factor receptor 2 status.

3. The method of claim 1, wherein the receptor gene status comprises a hormone receptor status.

4. The method of claim 1, further comprising applying the trained statistical model to the values for the one or more input features to obtain an output indicative of the receptor gene status.

5. The method of claim 1, wherein the sample type is indicative of a solid sample and the set of input features comprises genomic alteration features, complex mutational signatures, chromosomal instability features, clinicopathological features, clinical features, additional features, or a combination thereof.

6. The method of claim 5, wherein the genomic alteration features comprise one or more genomic alterations and a type of alteration associated with the one or more genomic alterations.

7. The method of claim 1, wherein the sample type is indicative of a liquid sample and the set of input features comprises genomic alteration features, clinicopathological features, clinical features, tumor fraction features, fragmentomic features, additional features, or a combination thereof.

8. (canceled)

9. The method of claim 1, wherein the subject is suspected of having or is determined to have cancer.

10. (canceled)

11. The method of claim 9, wherein the cancer comprises breast cancer (HER2 overexpressed/amplified), breast cancer (HER2+), breast cancer (HR+, HER2−), and prostate cancer.

12. The method of claim 11, further comprising treating the subject with an anti-cancer therapy.

13. The method of claim 12, wherein the anti-cancer therapy comprises alpelisib (Piqray), CDK4/6 inhibitors, or any combination thereof.

14. The method of claim 1, wherein the sample comprises a tissue biopsy sample, a liquid biopsy sample, or a normal control.

15. The method of claim 14, wherein the sample is a liquid biopsy sample and comprises circulating tumor cells (CTCs), cell-free DNA (cfDNA), circulating tumor DNA (ctDNA), or any combination thereof.

16. The method of claim 1, wherein the plurality of nucleic acid molecules comprises a mixture of tumor nucleic acid molecules and non-tumor nucleic acid molecules.

17. The method of claim 16, wherein the tumor nucleic acid molecules are derived from a tumor portion of a heterogeneous tissue biopsy sample, and the non-tumor nucleic acid molecules are derived from a normal portion of the heterogeneous tissue biopsy sample.

18. The method of claim 16, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

19. The method of claim 1, wherein the one or more adapters comprise amplification primers, flow cell adaptor sequences, substrate adapter sequences, or sample index sequences.

20. The method of claim 1, wherein the captured nucleic acid molecules are captured from the amplified nucleic acid molecules by hybridization to one or more bait molecules.

21. The method of claim 20, wherein the one or more bait molecules comprise one or more nucleic acid molecules, each comprising a region that is complementary to a region of a captured nucleic acid molecule.

22. (canceled)

23. The method of claim 1, wherein the sequencing comprises use of a massively parallel sequencing (MPS) technique, whole genome sequencing (WGS), whole exome sequencing, targeted sequencing, direct sequencing, or Sanger sequencing technique.

24.-31. (canceled)
